(19) United States
(12) Patent Application Publication
(10) Pub. No.: US 2019/0153409 A1
(43) Pub. Date: May 23, 2019

(54) COMPOSITIONS COMPRISING PARTICLES AND METHODS FOR TREATING CANCER

(71) Applicant: Aviv MedTech Ltd., Tel-Aviv (IL)

(72) Inventors: Nadir ARBER, Tel-Aviv (IL); Shiran SHAPIRA, Petach-Tikva (IL)

(73) Assignee: Aviv MedTech Ltd., Tel-Aviv (IL)

(21) Appl. No.: 16/191,667

(22) Filed: Nov. 15, 2018

Related U.S. Application Data
(60) Provisional application No. 62/587,483, filed on Nov. 17, 2017.

(51) Int. Cl. C12N 9/16; C07K 14/245; C07K 16/30; C07K 16/28; A61P 35/00

(52) U.S. Cl. CPC C12N 9/16; C12Y 301/27; C07K 14/245; C07K 2317/55; C07K 16/2896; A61P 35/00; C07K 2317/622; C07K 16/30

(57) ABSTRACT

A cell-free particle is provided. The cell-free particle comprising a nucleic acid sequence encoding a toxin and a nucleic acid sequence encoding an anti-toxin and wherein the particle comprises a targeting moiety for delivery of the particle into a cancer cell.

### RELATED APPLICATIONS

This application claims the benefit of priority under 35 USC § 119(e) of U.S. Provisional Patent Application No. 62/587,483 filed on Nov. 17, 2017, the contents of which are incorporated by reference as if fully set forth herein in their entirety.

### SEQUENCE LISTING STATEMENT

The ASCII file, entitled 75864_ST25.txt, created on Nov. 15, 2018 comprising 44,259 bytes, submitted concurrently with the filing of this application is incorporated herein by reference.

### FIELD AND BACKGROUND OF THE INVENTION

The present invention, in some embodiments thereof, relates to compositions comprising particles for treating cancer.

Colorectal cancer (CRC) is a major health concern in the Western world (American Cancer Society. Colorectal Cancer Facts & Figures. 2014). The prognosis for metastatic CRC still remains un-satisfactory. Resistance to chemotherapy is a major obstacle for effective treatment. CRC patients carrying KRAS (KRAS proto-oncogene, GTPase) mutations are a particular therapeutic challenge, due to their resistance to anti-EGFR therapies.

Aberrant activation of the RAS pathway plays an important role in the multistep process of CRC carcinogenesis. Oncogenic RAS stimulates a number of downstream effectors, that activate several transcription factors that bind to the RAS-responsive DNA element and induce early response gene expression. The polyoma (Py) virus enhancer consists flanking overlapping binding sites of the Ets and AP1 transcription factors that are essential for oncogene transcriptional activation (Reddy M A, et al., 1992).

Viral gene therapy is an innovative approach that offers a potential treatment for inherited and acquired diseases (Nabel et al., 2004). It usually involves the generation of replication defective viral particles that are capable of stably or transiently introducing a desirable transgene into cells, resulting in slowing down its' progression (Kootstra N A, et al., 2003; Verma I M, et al., 2005; Young L S, et al., 2006). The most characterized human adenoviruses of serotypes 2 and 5 (Ad2 and Ad5, respectively) usually cause mild upper respiratory tract infections, making them well suited for use in gene therapy.

Adenovirus-based cancer therapy are used for two main strategies: (i) direct tumor cell killing through delivery of replicating oncolytic viruses or non-replicating vectors encoding tumor suppressor genes, suicide genes or anti-angiogenic genes, (ii) destroy primary and metastatic cancer cells through induction of host antitumor immune responses (Kaplan et al., 2005). These approaches offer the potential of selective tumor cell destruction without damage to normal tissues. Apoptotic genes and tumor suppressor genes are used extensively in this field (El-Aneed et al., 2004), alone or in combination with chemotherapy. However, the ability to specifically target tumor cells with gene transfer is limited, and on the other hand, many normal (non-cancerous) cells are affected as well.

Previous studies have shown that recombinant adenovirus carrying the lethal gene PUMA (p53-upregulated modulator of apoptosis) (generous gift of Bert Vogelstein, Johns Hopkins University, Baltimore) under the control of Ets and AP1-RAS-responsive elements (Py2-SV40-PUMA) suppressed the growth of a variety of tumor cells harboring mutated RAS (Dvory-Sobol H, et al., 2005; Dvory-Sobol H, et al. 2006; Dvory-Sobol H, et al. 2007; Giladi N, et al. 2007; Naumov I, et al. 2012; FitzGerald D, et al., 1991).

The present inventors have also recently shown that the addition of multiple RAS-responsive elements (Py4/Py5-SV40-PUMA) further improved the growth inhibitory potency of the construct and induced apoptosis in CRC and pancreatic cancer cells in vitro and in vivo (Naumov I, et al. 2012; Lisiansky V, et al. 2012). However, escape mechanisms and increased expression of anti-apoptotic genes can render the cells resistant as the induced programmed cell death pathway can be inactivated.

MazF is a bacterial ribonuclease known to have specificity for ACA sequences in single-stranded RNA. MazF-induced toxicity is executed by blocking de novo protein synthesis through its endoribonuclease activity, termed mRNA interferases (Inouye et al., 2006). In nature, MazF is one of a pair of genes encoding for a stable toxin and an unstable antitoxin organized in a bicistronic operon as a part of a flexible genome (Pandey et al., 2005). The antitoxin interferes with the lethal action of the toxin and neutralizes its toxicity (Engelberg-Kulka H, et al., 2006; Engelberg-Kulka H, et al., 2005). This organization is a hallmark of toxin-antitoxin (TA) operons. TA systems are evolutionarily successful entities that are prevalent in lower organisms, bacteria and archaea, and they play important roles in a diverse range of cellular activities (Cook et al., 2013). Some TA systems might behave as selfish elements (found in plasmids), while others integrate into host regulatory networks (encoded from the chromosome). The first TA system to be identified was shown to play a role in plasmid maintenance (Thisted T, et al. 1994; 13:1960-8). Once a cell loses the plasmid encoding the TA system, the toxin will be released from the existing TA complex, given that the antitoxin is more unstable than the toxin. This results in cell growth inhibition that eventually leads to cell death (Gerdes K, et al., 1986).

Additional background art includes:
Shapira et al 2013 Cancer Res. 73:3303;
WO2016/185471;
Yu et al. Theranostics. 2012; 2(1): 3-44.

### SUMMARY OF THE INVENTION

According to an aspect of some embodiments of the present invention there is provided a cell-free particle comprising a nucleic acid sequence encoding a toxin and a nucleic acid sequence encoding an anti-toxin and wherein the particle comprises a targeting moiety for delivery of the particle into a cancer cell.

According to some embodiments of the invention, the cell-free particle comprises a nucleic acid construct comprising:

(i) a first nucleic acid sequence encoding the toxin operatively linked to a first promoter and at least one cancer-associated proliferative signaling responsive enhancer element;

(ii) a second nucleic acid sequence encoding the anti-toxin operatively linked to a second promoter, the second promoter being stronger than the first promoter.

According to some embodiments of the invention, the cell-free particle comprises a nucleic acid construct system comprising:

(i) a first nucleic acid construct encoding the toxin operatively linked to a first promoter and at least one cancer-associated signaling responsive enhancer element;

(ii) a second nucleic acid construct encoding the anti-toxin operatively linked to a second promoter, the second promoter being stronger than the first promoter.

According to some embodiments of the invention, the cell-free particle comprises a nucleic acid construct system comprising:

(i) a first nucleic acid construct encoding a toxin operatively linked to a first promoter and at least one cancer-associated signaling responsive enhancer element;

(ii) a second nucleic acid construct encoding an anti-toxin operatively linked to a second promoter;

wherein the first nucleic acid construct is provided at a higher concentration than the second nucleic acid construct.

According to some embodiments of the invention, the first promoter and the second promoter are identical promoters.

According to some embodiments of the invention, the toxin and the anti-toxin are selected from the group consisting of MazF/MazE, kid/kis, CcdB/CcdA, ChpBK/ChpBI, RelE/RelB, ParE/ParD, HipA/HipB, PhD/Doc, Hok/Sok, YafM/YoeB, YafN/YafO, YgjM/YgjN, YgiT/YgiU, DinJ/YafQ, VapB/VapC, HipB/HipA, and HicB/HicA.

According to some embodiments of the invention, the anti-toxin is translationally fused to a destabilization sequence.

According to some embodiments of the invention, the targeting moiety is selected from the group consisting of an antibody, an antibody fragment, a peptide and an aptamer.

According to some embodiments of the invention, the antibody or antibody fragment is encoded from a polynucleotide comprising a nucleic acid sequence encoding the antibody or antibody fragment translationally fused upstream of a nucleic acid sequence encoding a membrane-anchored amino acid sequence.

According to some embodiments of the invention, the membrane-anchored amino acid sequence comprises at least a partial transmembrane and an extracellular amino acid sequence of a retrovirus envelop glycoprotein.

According to some embodiments of the invention, the retrovirus is selected from the group consisting of a MuLV retrovirus and a lentivirus.

According to some embodiments of the invention, the retrovirus is a vesicular stomatitis virus (VSV).

According to some embodiments of the invention, the retrovirus is the VSV and wherein the envelop glycoprotein is set forth by SEQ ID NO: 58.

According to some embodiments of the invention, the retrovirus is the VSV and wherein the partial transmembrane and an extracellular amino acid sequence of the envelop glycoprotein is encoded by the nucleic acid sequence set forth by SEQ ID NO: 57.

According to some embodiments of the invention, the polynucleotide further comprises a nucleic acid sequence encoding a signal peptide being translationally fused upstream of the nucleic acid sequence encoding the antibody or the antibody fragment.

According to some embodiments of the invention, the targeting moiety specifically binds a tumor marker.

According to some embodiments of the invention, the tumor marker is selected from the group consisting of CD24, AFP, αᵥβ₃ (vitronectin receptor), CA125 (MUC16), CD4, CD20, CD22 (Siglec-2), CD30 (TNFRSF1), CD33 (Siglec-3), CD52 (CAMPATH-1), CD56 (NCAM), CD66e (CEA), CD80 (B7-1), CD140b (PDGFRβ), CD152 (CTLA4), CD227 (PEM, MUC1, mucin-1), EGFR (HER1, ErbB1), EpCam, GD3 ganglioside, HER2 (HER2/neu,ErbB2), PSMA, Sialyl Lewis, “VEGF, E-cad, CLDN7, FGFR2b, N-cad, Cad-11, FGFR2c, EGFR, FGFR1, FOLR1, IGF-I Ra, GLP1R, PDGFRa, PDGFRb, TNFRSF11b, EPHB6, VEGFR, ABCG2, CXCR4, CXCR7, integrin-αvβ3, SPARC, VCAM, ICAM and CD44.

According to some embodiments of the invention, the particle has a diameter of about 30-120 nm.

According to some embodiments of the invention, the cell-free particle is comprised in a cell free sample in which the majority of protein is comprised in cell-free particles comprising a plurality of the cell-free particle.

According to some embodiments of the invention, the cell-free particle is an exosome.

According to some embodiments of the invention, the cell-free particle is derived from a cell selected from the group consisting of a tumor cell, a stem cell, healthy cell, stably transfected cell.

According to some embodiments of the invention, the cell is a human cell.

According to some embodiments of the invention, the second promoter comprises CMV and the first promoter comprises SV40.

According to some embodiments of the invention, the cancer-associated signaling responsive enhancer element comprises a Ras-responsive element.

According to some embodiments of the invention, the Ras-responsive element comprises the Ets binding site and/or Ap-1 binding site.

According to some embodiments of the invention, the Ets binding site is set forth by SEQ ID NO:1.

According to some embodiments of the invention, the Ap-1 binding site is set forth by SEQ ID NO:2.

According to some embodiments of the invention, the Ras-responsive element comprises the PY2 sequence.

According to some embodiments of the invention, the Ras-responsive element comprises at least four repeats of the PY2 sequence.

According to some embodiments of the invention, the PY2 sequence is set forth by SEQ ID NO:3.

According to some embodiments of the invention, the first nucleic acid construct and the nucleic acid construct are co-transfected into cells at a 1 to 0.5 ratio, respectively.

According to some embodiments of the invention, the Ras comprises K-Ras.

According to some embodiments of the invention, the anti-toxin comprises an RNA silencing agent.

According to some embodiments of the invention, the toxin and the anti-toxin comprise a bacterial-derived toxin anti-toxin system.

According to some embodiments of the invention, the toxin anti-toxin system comprise a MazEF system.

According to some embodiments of the invention, the second nucleic acid sequence or the second nucleic acid construct further comprises a non-cancerous associated responsive element for regulating transcription of the anti-toxin.

According to some embodiments of the invention, the non-cancerous associated responsive element comprises the p53 wild type responsive element.

According to some embodiments of the invention, the p53 wild type responsive element is set forth by SEQ ID NO:14.

According to some embodiments of the invention, the second nucleic acid sequence or the second nucleic acid construct comprises at least 2 repeats of the non-cancerous associated responsive element.

According to some embodiments of the invention, the second nucleic acid sequence or the second nucleic acid construct comprises 17 repeats of the p53 wild type responsive element.

According to some embodiments of the invention, the first nucleic acid sequence or the first nucleic acid construct further comprises a repressor of a bacterial repressor-operator system, the repressor being under a transcriptional regulation of the cancer-associated signaling responsive enhancer element, and wherein the second nucleic acid sequence or construct comprises an operator of the bacterial repressor-operator system, such that expression of the repressor inhibits expression of the antitoxin.

According to some embodiments of the invention, the repressor comprises the Tetracycline repressor (Tet-R) sequence, and wherein the operator comprises the tetracycline operator sequence.

According to some embodiments of the invention, the operator comprises at least two repeats of the sequence tetracycline operator sequence.

According to some embodiments of the invention, the first nucleic acid sequence or the first nucleic acid construct comprises four repeats of the PY2 sequence set forth by SEQ ID NO:2 being upstream and operably linked to the SV40 minimal promoter region set forth by SEQ ID NO:4, and a toxin coding sequence being downstream of and transcriptionally regulated by said SV40 minimal promoter region.

According to some embodiments of the invention, the second nucleic acid sequence or the second nucleic acid construct comprises the p53 wild type responsive element set forth by SEQ ID NO:14 being upstream and operably linked to the SV40 minimal promoter region set forth by SEQ ID NO:4, and an antitoxin coding sequence being downstream of and transcriptionally regulated by said SV40 minimal promoter region.

According to some embodiments of the invention, the second nucleic acid sequence or the second nucleic acid construct comprises about 17 repeats of said p53 wild type responsive element.

According to some embodiments of the invention, the first nucleic acid sequence or the first nucleic acid construct comprises four repeats of the PY2 sequence set forth by SEQ ID NO:2 being upstream and operably linked to the SV40 minimal promoter region set forth by SEQ ID NO:4, a toxin coding sequence being downstream of and transcriptionally regulated by the SV40 minimal promoter region, an IRES sequence set forth by SEQ ID NO:7 being downstream and operably linked to the toxin coding sequence, and a Tetracycline repressor set forth by SEQ ID NO: 8 being downstream of and operably linked to the IRES sequence.

According to some embodiments of the invention, the second nucleic acid sequence or the second nucleic acid construct comprises a CMV minimal promoter which comprises two repeats of a tetracycline operator as set forth by SEQ ID NO:9 and an antitoxin coding sequence being downstream of and operably linked to the CMV minimal promoter.

According to some embodiments of the invention, the second nucleic acid sequence or the second nucleic acid construct comprises at least one copy of the p53 wild type responsive element set forth by SEQ ID NO:14.

According to some embodiments of the invention, the second nucleic acid sequence or the second nucleic acid construct comprises 17 copies of the p53 wild type responsive element, wherein the 17 copies of the p53 wild type responsive element are set forth in SEQ ID NO:15.

According to an aspect of some embodiments of the present invention there is provided a pharmaceutical composition comprising the cell-free particle of some embodiments of the invention and a pharmaceutically acceptable carrier or diluents.

According to an aspect of some embodiments of the present invention there is provided a method of treating cancer in a subject in need thereof, the method comprising administering to the subject a therapeutically effective amount of the pharmaceutical composition, wherein cancer cells of the subject are characterized by expression of a tumor marker to which the targeting moiety is directed, thereby treating cancer in the subject.

According to some embodiments of the invention, the cancer cells are characterized by hyper activity of the signaling as compared to non-cancerous cells of the same tissue, thereby treating the cancer.

According to an aspect of some embodiments of the present invention there is provided a composition comprising the cell-free particle for use in the treatment of cancer, wherein cancer cells of the cancer are characterized by expression of a tumor marker to which the targeting moiety is directed and optionally wherein the cancer cells are characterized by hyper activity of the signaling as compared to non-cancerous cells of the same tissue.

According to some embodiments of the invention, the cancer comprises colon cancer.

According to some embodiments of the invention, the cancer comprises lung cancer.

According to some embodiments of the invention, the cancer comprises pancreatic cancer.

According to some embodiments of the invention, the cancer comprises gastric cancer.

According to some embodiments of the invention, the tumor marker comprises CD24.

According to some embodiments of the invention, the cancer is characterized by a hyperactive RAS GTPase activity.

According to some embodiments of the invention, the RAS is a KRAS protein and wherein the hyperactive KRAS is caused by a G13D mutation in the KRAS protein set forth by SEQ ID NO:16.

According to some embodiments of the invention, the RAS is a NRAS protein and wherein the hyperactive NRAS is caused by a Q61K mutation in the NRAS protein set forth by SEQ ID NO:17.

According to some embodiments of the invention, the RAS is a HRAS protein and wherein the hyperactive HRAS is caused by a G12V mutation in the HRAS protein set forth by SEQ ID NO:18.

According to some embodiments of the invention, the method further comprises treating a subject having the cancer by a treatment selected from the group consisting of chemotherapy, biological therapy, radiotherapy, phototherapy, photodynamic therapy, surgery, nutritional therapy, ablative therapy, combined radiotherapy and chemotherapy, brachiotherapy, proton beam therapy, immunotherapy, cellular therapy and photon beam radiosurgical therapy.

According to some embodiments of the invention, the composition further comprises an agent suitable for a treatment selected from the group consisting of chemotherapy, biological therapy, photodynamic therapy, nutritional therapy, brachiotherapy, immunotherapy, and cellular therapy.

According to some embodiments of the invention, the cell-free particle further comprises an anti-cancer agent.

According to some embodiments of the invention, the anti-cancer agent is encapsulated in or conjugated to the particle.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-I depict MazF cassette. FIG. 1A—A schematic illustration of the mazF cassette. The RRE-activated MazF cassette was constructed by cloning several elements in the following order (from the N terminus): four repeats of the RAS responsive, Ets (SEQ ID NO:1) and AP-1 (SEQ ID NO:2) binding sites (termed “py2”, SEQ ID NO: 3); SV40 minimal promoter (SEQ ID NO:4); monomeric red fluorescence protein mCherry (SEQ ID NO:5); E. coli MazF ribonuclease (SEQ ID NO:6). FIGS. 1B-F—The toxic effect of the mazF-encoding viruses. 1×10⁴ HCT116 cells were seeded in 96-well plates in complete medium. Median dilutions of the MazF-encoding viruses starting from 25 MOI were added to the cells on the next day. FIGS. 1B-E—images from a light microscopy (FIGS. 1B-C) and from a fluorescent microscopy (FIGS. 1D-E) depicting cell survival (all of the microscope images are of the same magnification). FIG. 1F—a histogram depicting quantification of cell survival based on an enzymatic MTT assay 72 hours after infection. Each bar represents the mean±SD of a set of data determined in triplicates. FIGS. 1G-H—1×10⁵ cells were seeded in 12-well plates in complete medium and infected with the different adenoviruses in 20 MOI for 72 hours. Cell death was measured by FACS after staining with Annexin V (FIG. 1G) and RedDot2 (FIG. 1H) dyes. FIG. 1I—a histogram depicting reporter gene expression (luciferase) under the transcriptional regulation of the SV40 promoter and the PY4 Ras-responsive element in HCT116 cells. Briefly, a nucleic acid construct which comprises the PY4 Ras-responsive element upstream of the SV40 promoter being operably linked to a luciferase (reporter gene) coding sequence was used for transfection of HCT116 cells in which Ras is hyperactive. 5×10⁵ HCT116 cells were seeded in 6-well plates. The next day, when the cells were about 50% confluent, co-transfection with 3μg (microgram) of PY4-SV40-LUC vector plus 0.3 ng (nanogram) of pRL-CMV (Promega) was performed using jetPEI™ (Polyplus-transfection Inc, NY, USA) according to the manufacturer's instructions. The Luc activity was normalized to Renilla Luc activity from a parallel co-transfection.

FIGS. 2A-E depict the MazEF cassette. FIG. 2A-A schematic illustration of the mazEF cassette with the Ras-responsive elements PY2. This construct is also referred to as “PY4-TA” or “pAdEasy-Py4-TA” hereinafter. The Ras-responsive element (RRE)-activated MazEF cassette was constructed by cloning several elements in the following order (from the N-terminus to the C-terminus): four repeats of “Py2” [comprising the Ras responsive Ets (SEQ ID NO:1) and AP-1 (SEQ ID NO:2) binding sites]; SV40 minimal promoter (SEQ ID NO:4); monomeric red fluorescence protein mCherry coding sequence (SEQ ID NO:5); E. coli MazF ribonuclease (SEQ ID NO:6); internal ribosome entry sites (“IRES”, SEQ ID NO: 7); tetracycline repressor coding sequence (SEQ ID NO:8); CMV minimal promoter with two copies of the tetracycline operator (SEQ ID NO:9; the sequence of the tetracycline operator is provided in SEQ ID NO:11); green fluorescence protein coding sequence (SEQ ID NO:12); E. coli antitoxin MazE coding sequence (SEQ ID NO:13). FIG. 2B depicts the same construct as in FIG. 2A, yet devoid of the Ras-responsive element Py2. This construct is also referred to as “ΔPY4-TA” hereinafter). FIG. 2C—A schematic illustration depicting the proposed mode of action of the PY4-TA construct (depicted in FIG. 1A) in cells characterized by a hyperactive Ras. The PY2 elements bind transcription factors in Ras hyperactive cells, leading to expression of the mazF toxin and the tetracycline repressor (TetR; shown in pink), which then binds to the tetracycline operator and interferes (e.g., blocks) with the expression the maze antitoxin under the control of the CMV minimal promoter. As a result, the expression of the mazF toxin is higher than the expression of the mazE antitoxin and the cells are doomed to die. FIG. 2D—A schematic illustration depicting the proposed mode of action of the PY4-TA construct (depicted in FIG. 1A) in cells characterized by a wild type Ras (i.e., not hyperactive Ras). Since the cells do not include an hyperactive Ras, the PY2 sites do not activate the transcription of the mazF toxin, nor the expression of the TetR which is downstream of the mazF toxin coding sequence, and as a result, the activity of MazF is inhibited as compared to the activity of the antitoxin mazE, thus ensuring the survival of cells having wild type Ras. FIG. 2E—Western blot analysis depicting expression of the reporter proteins GFP (which is translationally fused to the antitoxin mazE coding sequence) and mCherry (which is translationally fused to the toxin mazF coding sequence) in cells characterized by a hyperactive Ras at increasing MOIs (multiple of infection). Thus, when the MOI equals to “1” (a single virus particle infecting a single cell) there is only expression of the mCherry reporter protein, indicating expression of only the mazF toxin. At increasing MOI to “5” (5 virus particles infecting a single cell), there is also “leakage” of expression of the GFP reporter protein, indicating some degree of expression of the antitoxin maze, yet the expression of the mCherry is significantly higher.

FIGS. 3A-K—Cells having wild type Ras are protected from the cytotoxicity of MazF due to MazE expression. HT29 cells, with WT (wild type) RAS, were seeded in 96-well plates. After 24 hours, two-fold dilutions of recombinant adenoviruses encoding for MazF or MazEF were added for 72 hours. Qualitative examination was performed using light microscopy (left images in each of FIGS. 3A-K) and fluorescence microscopy showing expression of mCherry (red fluorescent, middle images in each of FIGS. 3A-K) or GFP (green fluorescent, right images in each of FIGS. 3A-K). Microscopic Magnification×100. FIG. 3A—uninfected cells; FIGS. 3B-C—cells were infected with 1.56 MOI of recombinant adenoviruses harboring the MazEF vector (FIG. 3B) or the MazF vector (FIG. 3C). FIGS. 3D-E—cells were infected with 3.12 MOI of recombinant adenoviruses harboring the MazEF vector (FIG. 3D) or the MazF vector (FIG. 3E). FIGS. 3F-G—cells were infected with 6.25 MOI of recombinant adenoviruses harboring the MazEF vector (FIG. 3F) or the MazF vector (FIG. 3G). FIGS. 3H-I—cells were infected with 12.5 MOI of recombinant adenoviruses harboring the MazEF vector (FIG. 3H) or the MazF vector (FIG. 3I). FIGS. 3J-K—cells were infected with 25 MOI of recombinant adenoviruses harboring the MazEF vector (FIG. 3J) or the MazF vector (FIG. 3K). Note the decrease in expression of mCherry in cells infected with the MazF vector as compared to the expression of mCherry in cells infected with the MazEF vector. Also note the inhibition of cell growth in cells infected with the MazF vector as compared to the cell growth in cells infected with the MazEF vector. When visualized under a fluorescence microscope, the intoxicated Ad-Py4-SV40-mCherry-MazF-infected cells showed very faint red fluorescence, indicating inefficient mCherry-MazF accumulation. This is due to the ribonuclease activity of MazF that results in inhibition of protein synthesis, including its own (Zhang Y, MazF cleaves cellular mRNAs specifically at ACA to block protein synthesis in Escherichia coli. Mol Cell 2003; 12:913-23′ Shapira A, Removal of hepatitis C virus-infected cells by a zymogenized bacterial toxin. PLoS One 2012; 7:e32320). On the other hand, the ribonuclease activity of MazF was neutralized by its antidote MazE in cells infected with pAdEasy-Py4-TA, as indicated by the presence of both red and green fluorescence.

FIGS. 4A-G depict eradication of R1 cells by recombinant adenovirus-mediated delivery of the PY4-TA (which includes the MazF-MazE) encoding cassette. FIG. 4A—A schematic illustration depicting the construction of the mCherry control cassette by cloning the monomeric red fluorescence protein mCherry (SEQ ID NO:5) downstream to the CMV promoter. 1×10⁴ R1 cells were seeded in 96-well plates. After 24 hours, two-fold dilutions of recombinant adenoviruses encoding for PY4-TA (MazF-MazE) or mCherry were added for 72 hours. FIGS. 4B-D—Representative pictures of uninfected cells (control, FIG. 4B) and cells that were infected with the PY4-TA cassette with 5 MOI (FIG. 4C) or 10 MOI (FIG. 4D) (magnification of ×100 in all microscopic images). FIG. 4E—A histogram depicting quantification of enzymatic MTT viability assay which were performed 72 hours post-infection. Cell survival was measured in R1 cells infected with mCherry (black bars, “R1 mCherry”) or the PY4-TA (white bars, “R1 TA”) at the infected MOI. The relative fraction of viable cells (relative to uninfected controls) was determined by MTT assay. Each bar represents the mean±SD of a set of data determined in triplicates. FIGS. 4F-G—fluorescence microscopic examination of the infected cells (5 MOI of PY4-TA encoding viruses) showing expression of mCherry (FIG. 4F, red staining) and GFP (FIG. 4G, green staining) of the same microscopic field.

FIGS. 5A-J depict a colony formation assay showing selective eradication of CRC cells by recombinant adenovirus-mediated delivery of the mazEF encoding cassette. On the day before infection, 5×10⁵ HCT116 (which include an hyperactive Ras) and HT29 (which include wild type Ras) cells were seeded in 6-well plates and subsequently infected with 25 and 10 MOI of the viruses “pAdEasy-Py4-mCherry-MazF-IRES-TetR-CMVmp (with the Tet operator)-MazE-IRES-EGFP” (labeled as “PY4-TA”) and “pAdEasy-SV40-mCherry-MazF-IRES-TetR-CMVmp-MazE-IRES-EGFP” (labeled as “ΔPY4-TA”) or left un-infected. After 7 hours, the cells were trypsinized and seeded at 3-fold dilutions and incubated for 7 days. Surviving colonies were stained with 0.02% crystal violet. Note the significant decrease in survival cells treated with the PY4-TA vector at both MOI concentrations in HCT116 cells as compared to the cells treated with the ΔPY4-TA vector. Also, it is noted that the cell growth of the HT29 cells were not affected by treatment with either one of the vectors, thus showing the specificity of the PY4-TA vectors for killing cancer cells such as HCT116 cells with hyperactive Ras and not HT29 cells which have wild type KRAS gene.

FIGS. 6A-P—1×10⁴ HCT116 and HT29 cells were seeded in 96-well plates. After 24 hours, two-fold dilutions of recombinant adenoviruses PY4-TA encoding for mazEF were added. FIGS. 6A-D—Microscopic examination of the uninfected cells. FIGS. 6A-B—HCT116 cells; FIGS. 6C-D—HT29 cells. FIGS. 6E-J—Microscopic examination of the infected cells (10 MOI) was performed 72 hours post-infection. FIGS. 6E-G—HCT116 cells. FIGS. 6H-J—HT29 cells. Shown is a light microscopy (FIGS. 6E and 6H), and fluorescent microscopy showing mCherry expression (FIGS. 6F and 6I) and GFP expression (FIGS. 6G and 6J). Note the significant decrease in mCherry and GFP expression in HCT116 cells treated with the PY4-TA (mazEF) vector as compared to HT29 cells treated with the same PY4-TA (mazEF) vector. FIG. 6K—A histogram depicting cell survival of HCT116 cells (empty bars) or HT29 cells (black bars) treated with the same PY4-TA (mazEF) vector at various MOI. Cell survival was determined by an enzymatic MTT viability assay, and the relative fraction of viable cells (relative to uninfected controls) was determined. Each bar represents the mean±SD of a set of data determined in triplicates. FIGS. 6L-O—HT29 cells were infected (10 MOI) in the presence (FIGS. 6N and 6O) or absence (FIGS. 6L and 6M) of 1 μg/ml tetracycline. The cells were examined using a microscope 72 hour post-infection. FIG. 6P—1×10⁴ HCT116 cells (having hyperactive Ras) were seeded in 96-well plates. The Ad-Py4-TA encoding viruses were added in several MOIs with (empty bars) or without (filled bars) 1 μg/ml tetracycline 24 hours later. The enzymatic MTT viability assay was performed after 72 hours. Size bars=200 μm in all microscopic images shown in FIGS. 6A-J; the images shown in FIGS. 6L-O were taken using the same magnification as those in FIGS. 6A-J).

FIGS. 7A-C depict FACS analyses (FIGS. 7A-B) and cell survival (FIG. 7C) of cells infected with the PY4-TA or ΔPY4-TA vectors. 1×10⁵ HCT116 cells were seeded in 12-well plates in complete medium and infected with the different adenoviruses at 10 MOI for 72 hours. FIGS. 7A-B—FACS analyses showing cell death after staining with Annexin V (FIG. 7A) or RedDot2 (FIG. 7B) dyes. In both analyses (FIGS. 7A-B) the untreated cells are shown in red, the toxin antitoxin-treated cells (infected with the PY4-TA vector) are shown in black, and the cells that were treated with the RRE deletion cassette (ΔPY4-TA) are shown in green. Note the increase in cell death in cells treated with the PY4-TA vector as compared to cells treated with the control ΔPY4-TA vector devoid of the PY4 elements. FIG. 7C—A histogram depicting cell survival following infection with the PY4-TA vector or the ΔPY4-TA vector. 1×10⁴HCT116 cells were seeded in 96-well plates. After 24 hours, two-fold dilutions of recombinant adenoviruses encoding for Py4-TA (empty bars) or ΔPy4-TA (filled bars) were added for 72 hours. The relative fraction of viable cells (relative to uninfected controls) was determined by MTT assay. Each bar represents the mean±SD of a set of data determined in triplicates.

FIGS. 8A-C depict inhibition of tumor growth in mice. Tumors were formed in nude mice by subcutaneous injection of 5×10⁶ HCT116 cells on day 0 and were treated twice with intraperitoneal 2×10⁹ PFU/mouse of the indicated viruses. FIG. 8A—A graph depicting fold increase of tumor size in mice treated with the various viruses. Tumor size was measured at the indicated time points and tumor volumes were calculated. The mean values for each group are shown, and the standard deviation is represented by error bars for each measurement. The P values for the ΔPy4-TA group compared to the PBS group are shown in red and those for the Py4-TA group compared to the PBS group are shown in green. Each bar represents the mean±SD of a set of data determined from six mice. FIGS. 8B-C—Imaging was performed on the living organism with the Maestro CRi imaging device (FIG. 8B) and outside the mouse body (FIG. 8C). The red fluorescence dye represents the expression of MazF and the green fluorescence dye represents the expression of MazE.

FIGS. 9A-C depict the cloning of a mutated KRAS. Mouse cells were stably transfected with the plasmid described in FIG. 9A, thus expressing the mutated KRAS in their genome resulting in a hyperactive ras in the cells. The cells were then transiently transfected with the luciferase construct described in FIG. 9B to test for clones having increased luciferase expression as a result of the binding of the PY4 ras enhancer elements to the transcription factors downstream of the KRAS hyperactive signaling pathway in the mouse cells. FIG. 9A—Schematic illustration of the KRAS G13D cassette. The mutated kras cassette was constructed by cloning of the KRAS gene [mutation in the coding sequence of amino acid at position 13 in which G (glycine) was replaced by D (aspartic acid)] downstream to the CMV promoter. IRES-GFP sequences were cloned downstream to the KRAS gene. FIG. 9B—Schematic illustration of a construct in which the luciferase coding sequence (SEQ ID NO:20) is under the regulation of the PY4 ras enhancer elements and the SV40 minimal promoter. FIG. 9C—A histogram depicting the fold increase of luciferase expression in the various clones. It is noted that clone C3 exhibits the highest expression of luciferase.

FIG. 10 is a Western blot analysis depicting the expression of GFP in tumors infected with AAV6 particles. Tumors were induced (derived from HT29 cell line) in nude mice. Then a systemic single infecting of the various serotypes in several titers was conducted, and after two weeks the mice were sacrificed, the tumors were removed and the expression of the GFP was evaluated by Western blot analysis.

FIGS. 11A-B are schematic illustrations of nucleic acid construct systems according to some embodiments of the invention. FIG. 11A—Shown in a dual system based on the Ras and p53 responsive elements. The first nucleic acid construct (Ad-PY4-mCherry-mazF) comprises 4 repeats of the PY2 ras enhancer element, followed by the SV40 minimal promoter, followed by the mCherry coding sequence and the mazF toxin; and the second nucleic acid construct (Ad-RGCX17-mazE-GFP) comprises the p53 wild type responsive element, followed by the SV40 minimal promoter, followed by the mazE antitoxin coding sequence and the GFP fluorescence reporter gene. FIG. 11B—Shown in a dual system based on the Ras and p53 responsive elements. The first nucleic acid construct (Ad-PY4-mCherry-mazF) comprises 4 repeats of the PY2 ras enhancer element, followed by the SV40 minimal promoter, followed by the mCherry coding sequence and the mazF toxin; and the second nucleic acid construct (Ad-RGCX17-CMV-mazE-GFP) comprises the p53 wild type responsive element, followed by the CMV minimal promoter (without the Tet operator), followed by the mazE antitoxin coding sequence and the GFP fluorescence reporter gene.

FIG. 12 is a histogram depicting cell viability (determined by an MTT assay) of lung cancer cell lines after co-infection with MazF and MazE in an MOI ratio of 1:0.5, respectively. It should be noted that in this experiment both the MazF and MazE constructs were under the transcriptional regulation of the SV40 minimal promoter. H1975 cells: Ras^{wt}/p53^{mut}; H1650 cells: Ras^{wt}/p53^{wt}; H2030 cells: Ras^{mut}/p53^{wt}; SHP77 cells: Ras^{mut}/p53^{mut}.

FIGS. 13A-B are histograms depicting luciferase assay (FIG. 13A) and an MTT assay (FIG. 13B). FIG. 13A—The activity of the PY4 Ras-responsive element was tested in H1299 (NRAS oncogene expressing cell line), A549 (KRAS oncogene expressing cell line) and T24 (HRAS oncogene expressing cell line). The cells were co-transfected with PY4-luciferase and pRL-CMV (Promega) plasmids. The Luciferase activity was normalized to Renilla Luc activity from a parallel co-transfection. The results show that PY4 transcription can be activated by 3 Ras variants. FIG. 13B—1×10⁴ H1299, H2030, A549, and T24 cells were seeded in 96-wells plates. On the next day, cells were infected with Ad-PY4-mCherry-mazF carrying viruses for 72 hours. Cell viability was measured by the MTT assay. The results show that toxin transcription can be activated by 3 Ras variants.

FIGS. 14A-C are Western blot analyses of p53 (FIG. 14A), p21 (FIG. 14B) and Tubulin (FIG. 14C) of HCT116 cells after treatment with SFU. HCT116 cells were treated with 50 μM 5FU for 24 hours. Then, total cell lysate was prepared and subjected to Western blot analysis for p53 (FIG. 14A) and p21 (FIG. 14B) analysis. Tubulin (FIG. 14C) was used as a loading control and in the analyses of both proteins for normalization.

FIGS. 15A-B depict mRNA levels of various transcripts following treatment of HCT116 cells with 5FU. HCT116 cells were treated with 50 μM 5FU for 24 hours. Then, RNA was prepared and used as a template for cDNA and semi-quantitative PCR was performed for the following transcripts: p21, Bax, Noxa, Puma, MDM2, 14-3-3o, CD95, Btg2, GADD45, and Survivin. The graph represents the quantification of mRNA levels performed using the primers listed in Table 3 in the EXAMPLES section which follows.

FIGS. 16A-H depict crystal violet analysis of cells infected with the viruses according to some embodiments of the invention. 5×10⁵ A549 cells (KRAS nut, p53 wild type; FIGS. 16A-D) and H1650 cells (KRAS wild type, p53 wild type) were seeded in 6-well plates. After 24 hours, the cells were infected with 10 MOI of the PY4-mazF-mCherry and RGC-mazE-GFP viruses, in a ratio of 1:0.5, respectively (FIGS. 16A and 16C for A549 cells; and FIG. 16E for H1650 cells). In parallel those cell lines were infected with ΔPY4-mazF-mcherry and RGC-mazE-GFP viruses, in a ratio of 1:0.5, respectively (FIGS. 16B and 16D for A549 cells; and FIG. 16F for H1650 cells). The CMV-mCherry vector was used as a control (FIG. 16G). After 7 hours, the cells were trypsinized and seeded in 3-fold dilutions and incubated for 7 days. Surviving colonies were fixed with 4% formaldehyde in PBS and stained with 0.02% crystal violet.

FIG. 17 is a histogram depicting the efficacy of mazF as evaluated in pancreatic cancer cells. PANC1, Mia Paca2, Colo357 (KRAS mutated cells) and BxPC3 (wild type RAS) cell lines were seeded in 96-well plates. After 24 hours, median dilutions of PY4-mazF-mcherry viruses were added. 72 hours later, cell survival was measured the enzymatic MTT assay. Note that the mazF toxin causes to selective eradication of KRAS mutated pancreatic cells. % of cell viability was significantly lower in the three mutated cell lines as compared to the wild type (wt) Kras cells.

FIG. 18 depicts a polynucleotide sequence (SEQ ID NO: 54) according to some embodiments of the invention comprising a nucleic acid sequence encoding a signal peptide (cyan highlight; SEQ ID NO: 55) being upstream and in-frame with a nucleic acid sequence encoding a CD24 scFV (yellow highlight; SEQ ID NO: 56) being upstream and in-frame with a nucleic acid sequence encoding a minimal sequence of the VSV-G envelop membrane glycoprotein (magenta highlight, SEQ ID NO: 57).

FIG. 19 is a schematic illustration depicting a method of selecting cells expressing CD24 scFV antibody and generating targeted exosomes from these cells. A polynucleotide sequence comprising the CD24 scFV and the mVSV-G (e.g., the polynucleotide set forth by SEQ ID NO: 54) was cloned into the PCDNA4/TO vector and the vector is used to transform HEK293 or T-Rex-293 cells by stable transformation. Stable clones which display the CD24 scFv on their membrane are incubated with a recombinant CD24 protein that is fused to a biotin molecule. Then, a Streptavidin (SA)-conjugated Allophycocyanin (APC) antibody is added (which binds to the biotinylated CD24 that are bound to the CD24 scFV presenting cells) and the detection of cells expressing CD24 scFV antibody is done by Flow cytometry. HEK293 or T-Rex-293 stable clones which display on their membrane CD24 scFV produce exosomes (the targeted exosomes).

FIGS. 20A-B schematically describe a nucleic acid construct system according to some embodiments of the invention, which when interacting with a cancerous cell having a mutated K-Ras and mutated P53 the construct system leads to cell death (FIG. 20A) and when interacting with a non-cancerous cell having wild type (WT) Ras and wild type P53 the construct system leads to cell survival (FIG. 20B).

### DESCRIPTION OF SPECIFIC EMBODIMENTS OF THE INVENTION

The present invention, in some embodiments thereof, relates to a cell-free particle for treating cancer and, more particularly, but not exclusively, to pharmaceutical compositions and methods using same for treating cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

KRAS mutation is an early event in CRC carcinogenesis. The present inventors have uncovered that the hyperactive RAS pathway can be exploited, rather than inhibited in order to treat cancer. Thus, the present inventors devised a well-regulated toxin anti-toxin (TA) system derived from E. coli which enables selective control and efficient killing of tumor cells while sparing normal cells.

To improve delivery and specificity of targeting, the present inventors have now devised a platform based on cell-derived particles e.g., exosomes that can be manipulated such that they target the payload (toxin-anti-toxin) to the target cell specifically, thus mitigating any non-specific activity that can affect the non-target cells.

Thus, according to an aspect of the invention there is provided a cell-free particle comprising a nucleic acid sequence encoding a toxin and a nucleic acid sequence encoding an anti-toxin and wherein the particle comprises a targeting moiety for delivery of the particle into a cancer cell.

The term “cell-free particle” (also referred to herein as “the particle”) as used herein refers to a discrete entity that incorporates biological matter such as proteins and/or RNA. It will be appreciated that the particle of this aspect of the present invention is not a biological cell (i.e., intact cell) or a viral particle.

The particle may be derivable from a cell by any of several means, for example by secretion, budding or dispersal from the cell. For example, the particle may be produced, exuded, emitted or shed from the cell. Where the cell is in cell culture, the particle may be secreted into the cell culture medium.

According to a specific embodiment, the particle is not a synthetic particle in the sense that it is not composed of a biological membrane of a cell. Thus, according to a specific embodiment, the particle is not a liposome.

According to some embodiments of the invention, the particle is not a virus particle.

The cell from which the particle is derived can be any cell.

According to a specific embodiment, the cell is a tumor cell.

According to a specific embodiment, the cell is a eukaryotic (e.g., mammalian) cell.

According to a specific embodiment, the cell is a human cell.

According to a specific embodiment, the cell is a non-pathogenic cell (e.g., immune cell, liver cell, connective tissue cell (e.g., fibroblast), kidney cell, pancreatic cell, cardiomyocyte, neural cell, etc).

According to a specific embodiment, the cell is a stem cell (e.g., an induced pluripotent stem cell (iPSC), embryonic stem cell (ESC), mesenchymal stem cell (MSC), hematopoietic stem cell, neuronal stem cell).

According to a specific embodiment, the cell is a healthy cell e.g., non-cancerous cell.

According to a specific embodiment, the cell is an autologous cell.

According to a specific embodiment, the cell is a non-autologous cell (e.g., allogeneic or xenogeneic cell).

According to a specific embodiment, the cell is a primary cell.

According to a specific embodiment, the cell is a cell line (e.g., HEK293 human embryonic kidney cell line).

According to a specific embodiment, the cell-free particle is comprised in a preparation in which at least 80% of cellular protein from which the particle is derived is comprised in the cell-free particles. According to a specific embodiment, at least 80% of the cell-free particles in the preparation are homogeneous in terms of size and biological activity.

The particle may in particular comprise a vesicle. The particle may comprise an exosome. The particles described here may comprise any one or more of the properties of the exosomes described herein.

The particle may comprise a vesicle or a flattened sphere limited by a lipid bilayer. The particles may comprise diameters of 30-100 nm. The particles may be formed by inward budding of the endosomal membrane. The particles may have a density of about 1.13-1.19 g/ml and may float on sucrose gradients. The particles may be enriched in cholesterol and sphingomyelin, and lipid raft markers such as GM1, GM3, flotillin and the src protein kinase Lyn. They may comprise RNA, for example miRNA.

According to a particular embodiment, the particle is an exosome.

As used herein, the term “exosome” refers to an extracellular vesicle that is released from a cell upon fusion of a multivesicular body (MVB) with the plasma membrane.

The exosome may (a) have a size of between 30 nm (nanometer) and 120 nm (nanometer) as determined by electron microscopy; (b) comprises a complex of molecular weight >100 kDa (kilodalton), comprising proteins of <100 kDa; (c) comprises a complex of molecular weight >300 kDa, comprising proteins of <300 kDa; (d) comprises a complex of molecular weight >1000 kDa; (e) has a size of between 2 nm and 200 nm, as determined by filtration against a 0.2 pM filter and concentration against a membrane with a molecular weight cut-off of 10 kDa; or (f) a hydrodynamic radius of below 100 nm, as determined by laser diffraction or dynamic light scattering.

The particle may be isolatable from a cell or a cell conditioned medium (CM).

Exosomes can also be purified by ultracentrifugation of clarified conditioned media at 100,000×g. They can also be purified by ultracentrifugation into a sucrose cushion. GMP methods for exosome purification from dendritic cells have been described in J Immunol Methods. 2002; 270:211-226.

Exosomes can also be purified by differential filtration, through nylon membrane filters of defined pore size. A first filtration though a large pore size will retain cellular fragments and debris. A subsequent filtration through a smaller pore size will retain exosomes and purify them from smaller size contaminants.

Exemplary methods for isolating exosomes are described in the Examples section that follows.

As used herein, an isolated exosome is one which is physically separated from its natural environment. An isolated exosome may be physically separated, in whole or in part, from tissue or cells with which it naturally exists, e.g., stem cells, fibroblasts, and macrophages. In some embodiments of the disclosure, a composition of isolated exosomes may be free of cells or it may be free or substantially free of conditioned media. Typically, the isolated exosomes are provided at a higher concentration than exosomes present in un-manipulated conditioned media.

The particle may be isolated for example by being separated from non-associated components based on any property of the particle. For example, the particle may be isolated based on molecular weight, size, shape, composition or biological activity.

The conditioned medium may be filtered or concentrated or both during, prior to or subsequent to separation. For example, it may be filtered through a membrane, for example one with a size or molecular weight cut-off. It may be subject to tangential force filtration or ultrafiltration.

For example, filtration with a membrane of a suitable molecular weight or size cutoff, as described in the Assays for Molecular Weight elsewhere in this document, may be used.

The conditioned medium, optionally filtered or concentrated or both, may be subject to further separation means, such as column chromatography. For example, high performance liquid chromatography (HPLC) with various columns may be used. The columns may be size exclusion columns or binding columns.

One or more properties or biological activities of the particle may be used to track its activity during fractionation of the conditioned medium (CM). As an example, light scattering, refractive index, dynamic light scattering or UV-visible detectors may be used to follow the particles.

The particle may have a size of greater than 2 nm. The particle may have a size of greater than 5 nm (nanometer), 10 nm, 20 nm, 30 nm, 40 nm or 50 nm. The particle may have a size of greater than 100 nm, such as greater than 150 nm. The particle may have a size of substantially 200 nm or greater.

The particle or particles may have a range of sizes, such as between 2 nm to 20 nm, 2 nm to 50 nm, 2 nm to 100 nm, 2 nm to 150 nm or 2 nm to 200 nm. The particle or particles may have a size between 20 nm to 50 nm, 20 nm to 100 nm, 20 nm to 150 nm or 20 nm to 200 nm. The particle or particles may have a size between 50 nm to 100 nm, 50 nm to 150 nm or 50 nm to 200 nm. The particle or particles may have a size between 100 nm to 150 nm or 100 nm to 200 nm. The particle or particles may have a size between 150 nm to 200 nm.

According to some embodiments of the invention, the particle has a diameter of about 30-120 nm.

The size may be determined by various means. In principle, the size may be determined by size fractionation and filtration through a membrane with the relevant size cut-off. The particle size may then be determined by tracking segregation of component proteins with SDS-PAGE or by a biological assay.

The size may comprise a hydrodynamic radius. The hydrodynamic radius of the particle may be below 100 nm. It may be between about 30 nm and about 70 nm. The hydrodynamic radius may be between about 40 nm and about 60 nm, such as between about 45 nm and about 55 nm. The hydrodynamic radius may be about 50 nm.

The hydrodynamic radius of the particle may be determined by any suitable means, for example, laser diffraction or dynamic light scattering.

According to some embodiments of the invention, the cell-free particle is comprised in a cell free sample in which the majority of protein is comprised in cell-free particles comprising a plurality of the cell-free particle.

According to some embodiments of the invention, the cell-free particle is derived from a cell selected from the group consisting of a tumor cell, a stem cell, healthy cell, stably transfected cell.

According to some embodiments of the invention, the cell is a human cell.

As mentioned, the particle comprises (i.e., presents on the outer surface) a targeting moiety for delivery of the particle into a cancer cell.

Thus the particle may comprise an endogenous or exogenous affinity moiety to a cancer cell. Such a targeting moiety ensures specific delivery of the particle (and payload) to the cancer cells.

The targeting moiety may be monovalent or multivalent.

Particles that present the targeting moiety can bind to target cells through ligand-receptor interactions that induce receptor-mediated endocytosis and drug release inside the cell. Efficient binding and internalization requires that receptors are expressed exclusively on target cancer cells (10⁴-10⁵ copies per cell) relative to normal cells, and expression should be homogenous across all targeted cells. This delivery strategy achieves a high targeting specificity and delivery efficiency, while avoiding nonspecific binding and the MDR efflux mechanism. At present, several targeted delivery systems are under clinical trials, such as transferrin receptor targeted cytotoxic platinum-based oxaliplatin in a liposome (MBP-426), transferrin receptor targeted cyclodextrin-containing nanoparticles with siRNA payload (CALAA-01), or prostate-specific membrane antigen (PSMA) targeted polymeric nanoparticles containing docetaxel (BIND-014).

The targeting moiety can be attached to the surface of the particle by chemical means or genetic means. In the latter case, the cell from which the particles are derived is genetically manipulated to express the targeting moiety on the cell surface.

Examples of targeting moieties that can be used in accordance with the present teachings, include, but are not limited to antibodies, antibody fragment(s), peptides, aptamers and small molecules.

According to some embodiments of the invention, the antibody or antibody fragment is encoded by a polynucleotide comprising a nucleic acid sequence encoding the antibody or antibody fragment being translationally fused upstream of a nucleic acid sequence encoding a membrane-anchored amino acid sequence (an amino sequence which is within the membrane and can optionally be also presented on the outer surface of the membrane).

The phrases “translationally fused” and “in frame” are interchangeably used herein to refer to polynucleotides which are covalently linked to form a single continuous open reading frame spanning the length of the coding sequences of the linked polynucleotides. Such polynucleotides can be covalently linked directly or preferably indirectly through a spacer or linker region.

According to some embodiments of the invention, the membrane-anchored amino acid sequence comprises at least a partial transmembrane and an extracellular amino acid sequence of a retrovirus envelop glycoprotein.

According to some embodiments of the invention, the retrovirus is VSV (vesicular stomatitis virus) and wherein the envelop glycoprotein is set forth by SEQ ID NO: 58.

According to some embodiments of the invention, the retrovirus is VSV and wherein the partial transmembrane and an extracellular amino acid sequence of the envelop glycoprotein is encoded by the nucleic acid sequence set forth by SEQ ID NO: 57.

According to some embodiments of the invention, the polynucleotide further comprises a nucleic acid sequence encoding a signal peptide being translationally fused upstream of the nucleic acid sequence encoding the antibody or the antibody fragment.

The signal peptide can be for example of a protein which is designated for expression in the membrane, such as of CD24 antibody.

The term “antibody” as used in this invention includes intact molecules as well as functional fragments thereof (such as Fab, F(ab′)2, Fv, scFv, dsFv, or single domain molecules such as VH and VL) that are capable of binding to an epitope of an antigen.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Peptides are attractive targeting molecules due to their small size, low immunogenicity, and ease of manufacture at small costs. Peptide-based targeting ligands may be identified via several methods. Most commonly, they are obtained from the binding regions of a protein of interest. Phage display techniques can also be used to identify peptide-targeting ligands. In a phage display screen, bacteriophages present a variety of targeting peptide sequences in a phage display library (˜10¹¹ different sequences), and target peptides are selected using a binding assay. Cilengitide, a cyclic peptide with integrin binding affinity, is currently in phase II clinical trials for the treatment of non-small cell lung cancer and pancreatic cancer. In 2006, an Adnectin for human VEGF receptor 2 (Angiocept), a 40 amino acid thermostable and protease-stable oligopeptide, entered phase I clinical trials for the treatment of advanced solid tumors and non-Hodgkin's lymphoma. The affinity or stability of the peptides can be improved by using multivalent peptides and/or peptides that are composed of D-amino acids.

Small molecules with infinitely diverse structures and properties are inexpensive to produce and have great potential as a class of targeting moieties. One of the most widely studied small molecules as a targeting moiety for the delivery of agents is folic acid (folate). Folate is a water-soluble vitamin B6 and is essential in humans for rapid cell division and growth, especially during embryonic development. In cancers, folate receptors are overexpressed on tumor cells, so folate, which has a high binding affinity for the folate receptor (K_{d}=10⁻⁹ M) enables the targeted delivery of imaging and therapeutic agents to tumors. ¹¹¹In-DTPA-folate, ^{99m}Tc-folate conjugate (EC20), folate-linked fluorescent hepten (EC17), and diacetylvinylblastine hydrazide-folate conjugate (EC145) are currently being tested in clinical trials as cancer imaging agents and therapeutics. Folate has been combined with drug delivery vehicles or inorganic nanoparticles to produce targeted delivery of theranostic agents. Carbohydrates form another class of small molecule targeting ligands that selectively recognize cell surface receptors, such as lectin. The asialoglycoprotein receptor (ASGP-R) is present only on hepatocytes at a high density of 500,000 receptors per cell, and it readily binds carbohydrates, such as galactose, mannose, arabinose, which can thereby serve as effective liver-targeted drug delivery systems in vivo.

Aptamers are small nucleic acid ligands (15-40 bases) that bind to targets with high specificity due to the ability of the molecules to fold into unique conformations with three-dimensional structures. Aptamers are identified via a selection processes similar to phage display, involving the systematic evolution of ligands by exponential enrichment (SELEX). From libraries of 10¹⁵ random oligonucleotides, aptamers displaying high affinity and specificity for a target can be selected. This targeting moiety has potential advantage over antibodies, such as the small in size (15 kDa), low immunogenicity, and easy scale-up preparation without batch-to-batch variations. To date, more than 200 aptamers have been isolated. Pegaptanib, a VEGF₁₆₅ targeted aptamer, was approved by the FDA in 2004 for the treatment of neovascular macular degeneration. AS1411, a nucleolin targeted aptamer, is in phase II of clinical development.

A broad spectrum of chemical approaches can be used to conjugate targeting moieties, particle surfaces. These methods can be categorized as conventional bioconjugation strategies (direct conjugation, linker chemistry, physical interactions), click chemistry, or hybridization methods. The primary goal of targeted ligand conjugation is to bind a targeting moiety without losing its functionality after attachment to the particle.

Direct reaction strategies involve particle surface functionalization with amine, aldehyde, or active hydrogen groups.

Linker molecules on the other hand can control the binding orientations of ligands, thus, bioconjugation via linker chemistry is preferred over direct conjugation strategies for the attachment of targeting moieties to particles. Antibodies, peptides, and small molecules may be conjugated to particles using a variety of linkers. The most common linker chemistry relies on the reaction between amine-modified particles and sulfhydryl-containing biomolecules. Cysteine residues may be present or introduced into proteins and peptides, or the peptide may be chemically modified to gain this functionality.

For further details on various conjugation methods can be found in Yu et al. Theranostics. 2012; 2(1): 3-44, which is hereby incorporated by reference in its entirety.

According to a specific embodiment, the targeting moiety binds a molecule that is predominantly presented on a cancer cell of interest (and not in healthy tissues).

Such a molecule is referred to as a tumor marker that can be also a “tumor associated antigen”.

According to some embodiments of the invention, the targeting moiety specifically binds a tumor marker.

According to some embodiments of the invention, the tumor marker is selected from the group consisting of CD24, AFP, αᵥβ₃ (vitronectin receptor), CA125 (MUC16), CD4, CD20, CD22 (Siglec-2), CD30 (TNFRSF1), CD33 (Siglec-3), CD52 (CAMPATH-1), CD56 (NCAM), CD66e (CEA), CD80 (B7-1), CD140b (PDGFRβ), CD152 (CTLA4), CD227 (PEM, MUC1, mucin-1), EGFR (HER1, ErbB1), EpCam, GD3 ganglioside, HER2 (HER2/neu,ErbB2), PSMA, Sialyl Lewis, VEGF, E-cad, CLDN7, FGFR2b, N-cad, Cad-11, FGFR2c, EGFR, FGFR1, FOLR1, IGF-I Ra, GLP1R, PDGFRa, PDGFRb, TNFRSF11b, EPHB6, VEGFR, ABCG2, CXCR4, CXCR7, integrin-αvβ3, SPARC, VCAM, ICAM and CD44.

Table 1 below lists such tumor markers. The list aims not to be limiting but to merely provide exemplary embodiments.

TABLE 1

| Tumor marker | |
|---|---|
| (which can bind to the | |
| targeting moiety) | Tumor targets |
| CD24 | CRC, prostate, gastric, B-cell lymphomas, |
| | erythroleukemia, gliomas, small cell lung |
| | cancer, esophageal squamous cell |
| | carcinoma, hepatocellular carcinoma, |
| | cholangiocarcinoma, pancreatic |
| | adenocarcinoma, urothelial carcinoma, |
| | ovarian cancer, breast cancer, primary |
| | neuroendocrine carcinomas |
| AFP | Liver, germ cell |
| αᵥβ₃ (vitronectin receptor) | Melanoma |
| CA125 (MUC16) | Ovarian |
| CD4 | CTCL |
| CD20 | NHL |
| CD22 (Siglec-2) | NHL |
| CD30 (TNFRSF1) | HD, ALCL |
| CD33 (Siglec-3) | AML |
| CD52 (CAMPATH-1) | B-cell CLL |
| CD56 (NCAM) | SCLC |
| CD66e (CEA) | Metastatic CRC |
| CD80 (B7-1) | NHL |
| CD140b (PDGFRβ) | |
| CD152 (CTLA4) | Metastatic melanoma, prostate, breast |
| CD227 (PEM, MUC1, | Ovarian, gastric, MM |
| mucin-1) | |
| EGFR (HER1, ErbB1) | Metastatic CRC, CRC, renal cell, prostate, |
| | NSCLC, Head and neck, pediatric gliomas |
| EpCam | NSCLC, breast cancer, metastatic CRC |
| GD3 ganglioside | SCLC |
| HER2 (HER2/neu, ErbB2) | Metastatic breast overexpressing |
| | HER2/neu, prostate, ovarian, NSCLC, |
| PSMA | Prostate |
| Sialyl Lewis^{Y} | NSCLC |
| VEGF | Metastatic CRC |
| CLDN7 | Renal tumor, |
| FGFR2b | Breast cancer, gastric cancer |
| N-cad | Pancreatic cancer, CRC |
| Cad-11 | Pancreatic cancer, Prostate cancer |
| FGFR2c | Breast cancer |
| EGFR | NSCLC |
| FGFR1 | Prostate, SCLC, breast cancer, CRC |
| FOLR1 | Ovarian carcinoma, breast, brain, lung and |
| | colorectal cancers |
| GLP1R | Metastatic PDAC, pancreatic cancer, |
| | prostate cancer |
| PDGFRa | Osteosarcoma, NSCLC |
| PDGFRb | Breast, NSCLC |
| TNFRSF11b | Myeloma, breast, prostate, urothelial |
| | cancer, cervical cancer |
| EPHB6 | urothelial cancer, glioma, cervical cancer |
| VEGFR | Pancreatic cancer, CRC, ovarian, |
| | melanoma |
| ABCG2 | Cancer steam cells |
| CXCR4 | Breast, leukemia |
| CXCR7 | Bladder, breast |
| SPARC | Early PDAC, invasive meningioma, |
| | NSCLC, pancreatic cancer |
| VCAM | Gastric cancer, melanoma. Breast cancer |
| ICAM | Breast, cervical cancer, head and neck |
| CD44 | Ovarian, breast, colon, leukemia |

Table 1.

According to a specific embodiment, the tumor marker is CD24.

According to a specific embodiment, the tumor marker is CD24 and the targeting moiety is an anti-CD24 antibody.

Thus, the particles encapsulate nucleic acid sequences that express the toxin-anti-toxin agents as described herein.

Exosomes are first isolated and then the expression construct(s) are introduced thereto.

According to an embodiment, nucleic acid construct(s) encoding the toxin anti-toxin pair are introduced into exosomes via transfection such as with a commercial transfection reagent followed by DNase treatment to remove un-encapsulated DNA. Other transfection means can be employed such as electroporation, Xfect reagent and microinjection.

The term “target cells” as used in the context of this invention refers to cells that are treated with a toxin-antitoxin construct according to the invention, and that are targeted to diminish the cell growth rate of these cells or to kill them. In the target cells either the activity of the toxin is increased relative to the activity of the antitoxin, or the activity of the antitoxin is decreased relative to the activity of the toxin.

It should be noted that the target cell present the target protein to which the targeting moiety is directed.

According to a specific embodiment, the target cell is a pathogenic cell (e.g., cancer cell).

The term “non-target cells” as used in the context of this invention refers to cells that are treated with a toxin-antitoxin construct according to the invention, and that are targeted for protection against the toxic effects of the toxin of the toxin-antitoxin pair that diminishes the cell growth rate of these cells or kills them. In the non-target cells either the activity of the toxin is decreased relative to the activity of the antitoxin, or the activity of the antitoxin is increased relative to the activity of the toxin.

According to a specific embodiment, the non-target cell is a healthy cell e.g., non-cancerous cell.

In order to exhibit cytotoxic effects on target cells, it is necessary to either specifically kill the target cells or to protect non-target cells from the toxic effect of the toxin. This is achieved, first, by way of targeting specifically to the target cell with the targeting moiety as described hereinabove. Second, by way of designing the construct(s) such that the toxin is more active or expressed at higher amounts in the target cells; or alternatively or additionally, the anti-toxin is less stable or expressed to less amounts in the target cells as compared to the non-target cells.

There are numerous ways to achieve balanced expression of the toxin anti-toxin pair.

Some are described in US20140341930 which is hereby incorporated by reference, teaching a protein output modifier (POM) that is able to change the relative ratio in the concentration of the toxin and/or the antitoxin within the target cells and/or, where applicable, within non-target cells. According to a specific embodiment, the POM utilized is cell-specific, which is herein also termed as cs-POM (cell-specific protein output modifier). The protein output modifier (POM) according to the invention can be any compound or molecule that is able to change the concentration of the toxin-antitoxin substance within the targeted cell, either on the level of DNA, RNA or protein. In this respect, the protein output modifier (POM) modifies the relative rate of transcription, mRNA stability, mRNA translation stability or protein stability of the toxin and/or antitoxin. Depending on the kind of protein output modifier (POM) used, the level of modulation can be different and either affect transcription, translation or the stability of mRNA or the synthesized protein within the cell.

Diminishing cell growth or cell killing of target cells can hence be achieved by changing the relative ratio in the concentration of the toxin and/or the antitoxin, either in the target cells and/or in the non-target cells. There are several alternatives of how such a change in the ratio of the toxin relative to the antitoxin can be achieved. In one aspect the active antitoxin output within the cell can be decreased in the target cells, which would lead to an excess of the toxin over the antitoxin in the target cells, thereby inducing cell death or cell growth arrest. In addition or alternatively, the antitoxin output can be increased in the non-target cells relative to the toxin output, which would result in a protection of the non-target cells. In another aspect, the toxin output in the non-target cells can be decreased relative to the antitoxin output, resulting in a protection of the non-target cells. As a further alternative, the toxin output in the target cells can be increased, which would necessarily result in a killing of the target cells. Therefore, either killing of the target cells or protection of the non-target cells can be used as an approach for diminishing cell growth or inducing selective killing of pathogenic cells.

According to an alternative or an additional embodiment, the anti-toxin is translationally fused to a destabilizing sequence. Such sequences are described in details in US 20100196951, which is hereby incorporated by reference in its entirety.

Following is a description of specific embodiments of nucleic acid constructs and nucleic acid construct systems which can be used to express the toxin anti-toxin pairs.

According to some embodiments of the invention, the nucleic acid construct comprising:

(i) a first nucleic acid sequence encoding a toxin operatively linked to a first promoter and at least one cancer-associated signaling responsive enhancer element;

(ii) a second nucleic acid sequence encoding an anti-toxin operatively linked to a second promoter, the second promoter being stronger than the first promoter.

According to some embodiments of the invention, the nucleic acid construct system comprising:

(i) a first nucleic acid construct encoding a toxin operatively linked to a first promoter and at least one cancer-associated signaling responsive enhancer element;

(ii) a second nucleic acid construct encoding an anti-toxin operatively linked to a second promoter, the second promoter being stronger than the first promoter.

According to some embodiments of the invention, the first promoter and the second promoter are identical promoters.

According to some embodiments of the invention, the nucleic acid construct system comprising:

(i) a first nucleic acid construct encoding a toxin operatively linked to a first promoter and at least one cancer-associated signaling responsive enhancer element;

(ii) a second nucleic acid construct encoding an anti-toxin operatively linked to a second promoter;

wherein the first nucleic acid construct is provided at a higher concentration than the second nucleic acid construct.

As used herein the term “system” refers to at least two distinct nucleic acid construct molecules.

A coding nucleic acid sequence is “operably linked” or “operatively linked” (which is interchangeably used herein) to a regulatory sequence (e.g., promoter) if the regulatory sequence has a transcriptional regulatory effect on the coding sequence linked thereto.

As used herein, the term “promoter” refers to a region of DNA which lies upstream of the transcriptional initiation site of a gene to which RNA polymerase binds to initiate transcription of RNA. The promoter controls where (e.g., in which cells) and/or when (e.g., at which stage or condition in the lifetime of an organism) the gene is expressed.

The promoter can direct transcription of the polynucleotide sequence operably linked thereto in a constitutive or inducible manner.

According to some embodiments of the invention, the promoter is heterologous to the coding sequence operably linked thereto.

As used herein the phrase “heterologous promoter” refers to a promoter from a different gene locus as of the coding sequence operably linked thereto.

According to some embodiments of the invention, the promoter comprises the minimal promoter sequence required for transcription of the coding sequence operably linked to the promoter.

Assays for determining the minimal promoter sequence are known in the art, and described, for example, in Byrne B J., et al., 1983 [“Definition of the simian virus 40 early promoter region and demonstration of a host range bias in the enhancement effect of the simian virus 40 72-base-pair repeat. Proc Natl Acad Sci USA. 80(3): 721-725], which is fully incorporated herein by reference in its entirety.

For example, the minimal SV40 promoter sequence is set forth by SEQ ID NO:40; and the minimal CMV promoter sequence is set forth by SEQ ID NO: 19.

According to some embodiments of the invention, the second promoter is stronger than the first promoter.

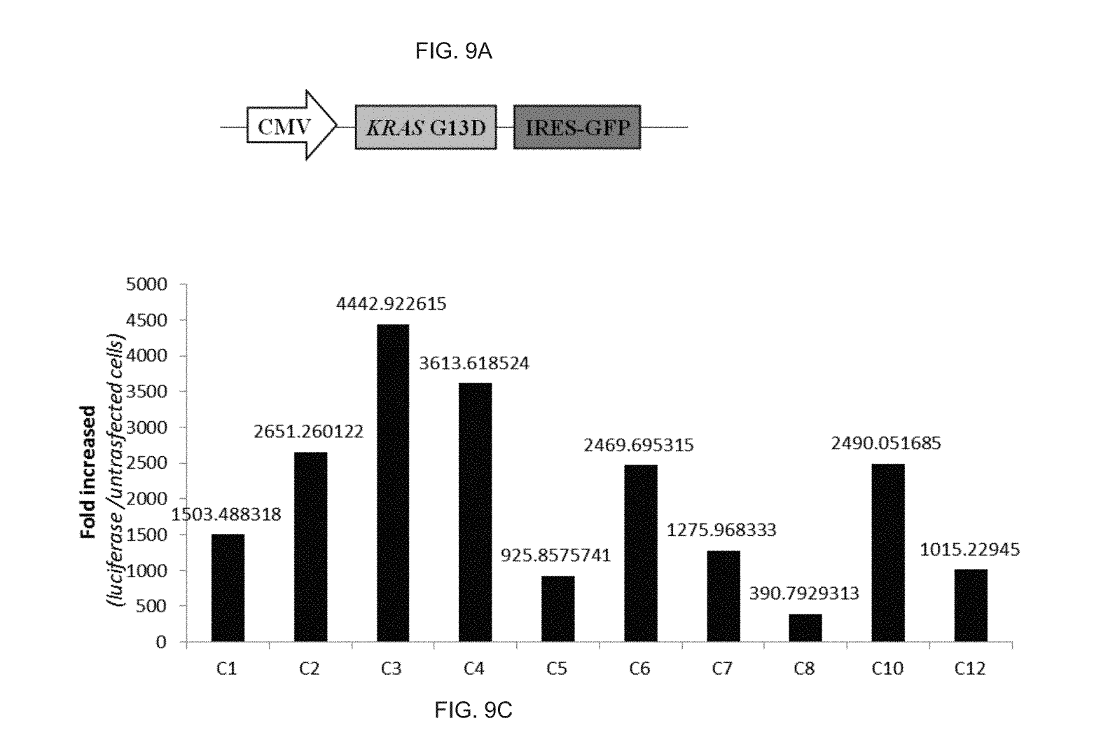

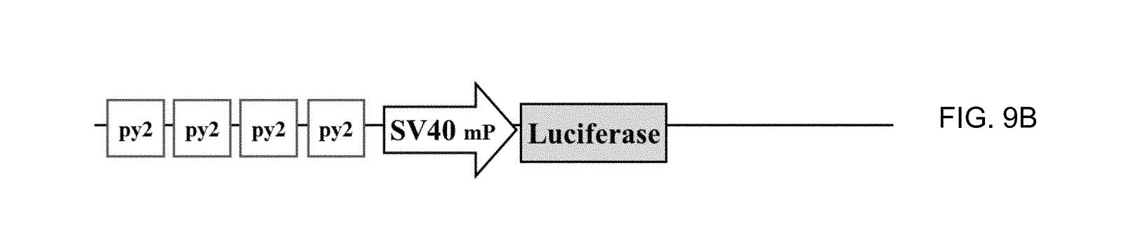

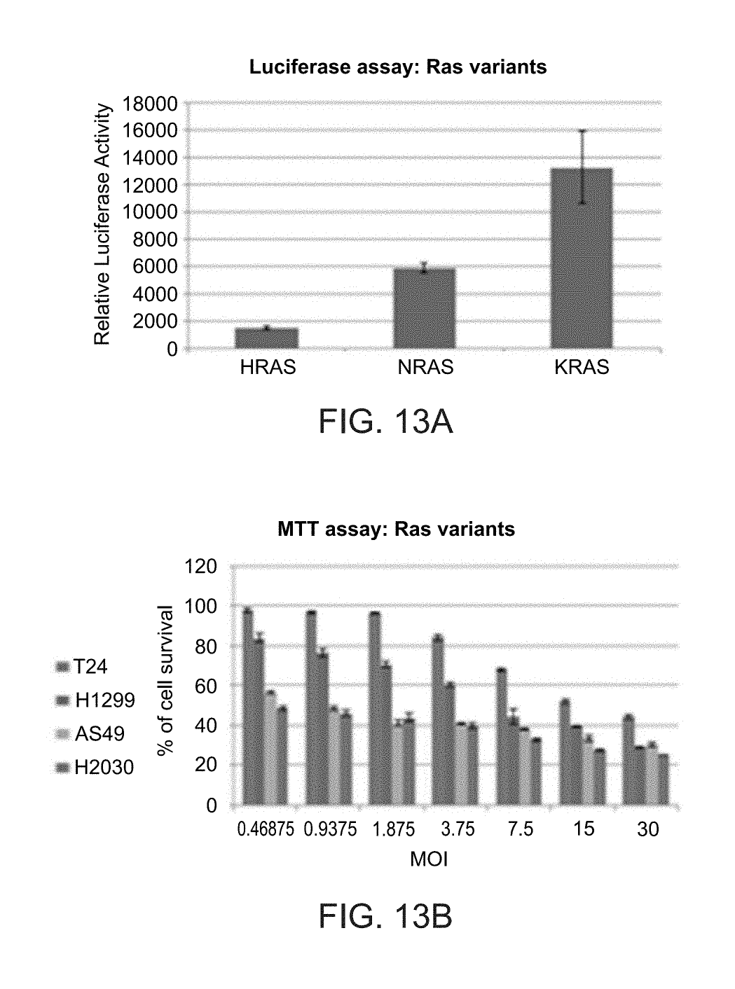

It should be noted that promoter activity can be detected and evaluated by various methods, such as by operably linking thereto a coding sequence of a reporter protein which can be detected and quantified in cells transfected with the construct. Examples of such assays include, but are not limited to using the luciferase coding sequence (e.g., SEQ ID NO: 20) under the control of the promoter to be tested, and measuring luciferase activity in the transfected cells (e.g., as described in FIGS. 9B and 13A). Thus, for example, selection of suitable first and second promoters can be performed by comparing the transcription ability of these promoters under identical assay conditions, using for example, the same reporter coding sequence.

According to some embodiments of the invention, the second promoter exhibits at least 10%, 20%, 30%, 40%, 50%, 60%, 70%, 80%, 90%, 100%, 150%, 200%, 300%, 400%, 500%, e.g., at least 1000%, higher transcription ability to transcribe a coding sequence operably linked thereto.

As used herein the phrase “cancer-associated signaling responsive enhancer element” refers to a nucleic acid sequence which serves as an enhancer of transcription by the binding of a specific transcription factor thereto, wherein the specific transcription factor is expressed in cancer cells.

Typically the cancer-associated signaling responsive enhancer element is a cancer-associated proliferative signaling responsive enhancer element.

Known proliferation signaling of cancer cells include the pathways used by several oncogenes. For example, in the RAS pathway the proliferation signaling results in binding of Ets and/or the AP-1 transcription factors to specific Ets and/or Ap-1 binding sites, respectively (which form the PY2 enhancer element). Similarly, in the WNT signaling pathway the proliferation signaling results in binding of the TCF/LEF transcription factors [e.g., TCF7 (TCF-1), TCF7L1 (TCF-3), TCF7L2 (TCF-4) and/or LEF1] to their binding sites (enhancers). Another example includes the MAPK pathway in which the transcription factor MYC (c-myc) can bind to specific binding sites as described elsewhere [Karen I. Zeller, et al., 2006. Global mapping of c-Myc binding sites and target gene networks in human B cells. Proc Natl Acad Sci USA; 103(47): 17834-17839, which is fully incorporated herein by reference].

Similarly, known proliferation signaling of cancer cells include the pathways used by tumor suppressor genes as in the case of retinoblastoma (Rb) tumor suppressor protein. The Rb tumor suppressor protein binds to the E2F1 transcription factor and prevents the interaction between E2F1 and the cell's transcription machinery. In the absence of Rb [e.g., when cyclin-dependent kinases (CDK) and cyclins phosphorylate Rb to pRb], E2F1 (along with its binding partner DP1) mediates the trans-activation of E2F1 target genes that facilitate the G1/S transition and S-phase. E2F targets genes encode proteins involved in DNA replication (for example DNA polymerase, thymidine kinase, dihydrofolate reductase and cdc6), and chromosomal replication (replication origin-binding protein HsOrc1 and MCMS). When cells are not proliferating, E2F DNA binding sites [(e.g., TTTCCCGC (SEQ ID NO:53)] contribute to transcriptional repression.

Thus, according to some embodiments of the invention the cancer-associated signaling responsive enhancer element comprises the E2F DNA binding site(s). In this case, when the nucleic acid construct or system thereof is introduced to cancer cells with a mutation in the Rb tumor suppressor (which prevents binding of Rb to E2F1) the E2F1 transcription factor in the cancer cells can bind to the E2F DNA binding sites (e.g., SEQ ID NO:53), resulting in activation of the promoter operably linked to the toxin coding sequence, and with high expression of the toxin within the cells.

According some embodiments of the invention, the cancer-associated proliferative signaling responsive enhancer element comprises a Ras-responsive element.

According some embodiments of the invention, the Ras comprises K-Ras.

According some embodiments of the invention, the Ras comprises H-Ras.

According some embodiments of the invention, the Ras comprises N-Ras.

According some embodiments of the invention, the Ras-responsive element comprises the Ets binding site and/or the Ap-1 binding site.

The Ets binding site comprises an ETS domain, which is a winged helix-turn-helix structure that binds to DNA sites with a central GGA(A/T) DNA sequence. The ETS family includes 12 subfamilies as described in Table 2 below.

TABLE 2

| Subfamily | Mammalian family members |
|---|---|
| ELF | ELF1, ELF2 (NERF), ELF4 (MEF) |
| ELG | GABPα |
| ERG | ERG, FLU, FEV |
| ERF | ERF (PE2), ETV3 (PE1) |
| ESE | ELF3 (ESE1/ESX), ELF5 (ESE2), ESE3 (EHF) |
| ETS | ETS1, ETS2 |
| PDEF | SPDEF (PDEF/PSE) |
| PEA3 | ETV4 (PEA3/E1AF), ETV5 (ERM), ETV1 (ER81) |
| ER71 | ETV2 (ER71) |
| SPI | SPI1 (PU.1), SPIB, SPIC |
| TCF | ELK1, ELK4 (SAP1), ELK3 (NET/SAP2) |
| TEL | ETV6 (TEL), ETV7 (TEL2) |

Table 2.

According some embodiments of the invention, the Ets binding site is set forth by SEQ ID NO:1.

According some embodiments of the invention, the AP-1 binding site is set forth by SEQ ID NO:2.

According some embodiments of the invention, the Ras-responsive element comprises the PY2 sequence. The PY2 sequence comprises the Ets and Ap-1 binding site and is set forth by SEQ ID NO: 3.

According some embodiments of the invention, the Ras-responsive element comprises at least two repeats of the PY2 sequence, e.g., at least three repeats of the PY2 sequence, e.g., at least four repeats of the PY2 sequence, e.g., at least five repeats of the PY2 sequence or more.

According some embodiments of the invention, the Ras-responsive element comprises four repeats of the PY2 sequence (the four repeats of the PY2 sequence are also referred to as “PY4” herein).

As used herein the term “toxin” refers to a polypeptide capable of killing cells.

As used herein the term “anti-toxin” (or “antitoxin”) refers to an RNA or a polypeptide capable of neutralizing the effect of the toxin in the cells where the toxin is present and/or active.

There are three known types of toxin-antitoxin systems. Type I toxin-antitoxin systems rely on the base-pairing of complementary antitoxin RNA with the toxin's mRNA. Translation of the mRNA is then inhibited either by degradation via RNase III or by occluding the Shine-Dalgarno sequence or ribosome binding site. Often the toxin and antitoxin are encoded on opposite strands of DNA. Known examples of type I toxin and anti-toxin pairs which can be used in the construct or system(s) of constructs according to some embodiments of the invention include, but are not limited to Hok-Sok [e.g., the Hok protein (SEQ ID NO:47; encoded by SEQ ID NO: 48) and the Sok coding sequence (SEQ ID NO: 49) encoding the SOK protein (SEQ ID NO:50)], fst-RNAII, TisB-IstR, LdrD-Rd1D, FlmA-FlmB, Ibs-Sib, TxpA/BrnT-RatA, SymE-SymR, and XCV2162-ptaRNA1 [reviewed in Sabine Brantl, and Natalie Jahn. sRNAs in bacterial type I and type III toxin-antitoxin systems. FEMS Microbiology Reviews. First published online: 25 Mar. 2015. doi: 10.1093/femsre/fuv003; Vogel J, et al., 2004. “The small RNA IstR inhibits synthesis of an SOS-induced toxic peptide”. Curr. Biol. 14 (24): 2271-6; Greenfield T J, et al., 2000. “The antisense RNA of the par locus of pAD1 regulates the expression of a 33-amino-acid toxic peptide by an unusual mechanism”. Mol. Microbiol. 37 (3): 652-60; Kawano M, et al., 2002. “Molecular characterization of long direct repeat (LDR) sequences expressing a stable mRNA encoding for a 35-amino-acid cell-killing peptide and a cis-encoded small antisense RNA in Escherichia coli”. Mol. Microbiol. 45 (2): 333-49; Loh S M, et al., 1988. “Nucleotide sequence and transcriptional analysis of a third function (Flm) involved in F-plasmid maintenance”. Gene 66 (2): 259-68; Fozo E M, et al., 2008. “Repression of small toxic protein synthesis by the Sib and OhsC small RNAs”. Mol. Microbiol. 70 (5): 1076-93; Silvaggi J M, et al. 2005. “Small Untranslated RNA Antitoxin in Bacillus subtilis”. J. Bacteriol. 187 (19): 6641-50; Gerdes K, et al. 2007. “RNA antitoxins”. Curr. Opin. Microbiol. 10 (2): 117-24; Findeiss S, et al. 2010. “A novel family of plasmid-transferred anti-sense ncRNAs”. RNA Biol. 7 (2): 120-4; each of which is fully incorporated herein by reference in its entirety].

In Type II toxin-antitoxin systems a labile protein antitoxin tightly binds and inhibits the activity of a stable toxin. Known examples of type II toxin and anti-toxin pairs which can be used in the construct or system(s) of constructs according to some embodiments of the invention include, but are not limited to, CcdB-CcdA, ParE-ParD, MazF-MazE, yafO-yafN, HicA-HicB, Kid-Kis, and Zeta-Epsilon [reviewed in Bahassi E M, et al., 1999. “Interactions of CcdB with DNA gyrase. Inactivation of Gyra, poisoning of the gyrase-DNA complex, and the antidote action of CcdA”. J Biol Chem 274 (16): 10936-44; Jensen R B, Gerdes K, 1995. “Programmed cell death in bacteria: proteic plasmid stabilization systems”. Mol Microbiol 17 (2): 205-10; Singletary L A, et al. 2009. “An SOS-Regulated Type 2 Toxin-Antitoxin System”. J. Bacteriol. 191 (24): 7456-65. doi:10.1128/JB.00963-09. PMC 2786605. PMID 19837801; Jørgensen M G et al. 2009. “HicA of Escherichia coli defines a novel family of translation-independent mRNA interferases in bacteria and archaea”. Journal of Bacteriology 191 (4): 1191-1199; Diago-Navarro E, et al., 2010. “parD toxin-antitoxin system of plasmid R1—basic contributions, biotechnological applications and relationships with closely-related toxin-antitoxin systems”. FEBS J. 277 (15): 3097-117; Mutschler H and Meinhart A. 2011. “ε/ζ systems: their role in resistance, virulence, and their potential for antibiotic development”. Journal of Molecular Medicine 89 (2): 1183-1194; each of which is fully incorporated herein by reference in its entirety].

Type III toxin-antitoxin systems rely on direct interaction between a toxic protein and an RNA antitoxin. The toxic effects of the protein are neutralized by the RNA gene. A non-limiting example of type III toxin and anti-toxin pairs which can be used in the construct or system(s) of constructs according to some embodiments of the invention is the ToxIN system from the bacterial plant pathogen Erwinia carotovora. The toxic ToxN protein is approximately 170 amino acids long and has been shown to be toxic to E. coli. The toxic activity of ToxN is inhibited by ToxI RNA, an RNA with 5.5 direct repeats of a 36 nucleotide motif (AGGTGATTTGCTACCTTTAAGTGCAGCTAGAAATTC, SEQ ID NO:24).

The sequences of the various toxin and anti-toxin agents (e.g., RNA or proteins) are known in the art and can be obtained from various sources including the “National Center for Biotechnology Information” data base [www(dot)ncbi(dot)nlm(dot)nih(dot)gov/].

According some embodiments of the invention, the anti-toxin is a polypeptide capable of neutralizing the effect of the toxin in the cells where the toxin is present and/or active.

According some embodiments of the invention, the anti-toxin comprises an RNA silencing agent.

According some embodiments of the invention, the toxin and the anti-toxin comprise a bacterial-derived toxin anti-toxin system.

According to some embodiments of the invention, the toxin and the anti-toxin are selected from the group consisting of MazF/MazE, kid/kis, CcdB/CcdA, ChpBK/ChpBI, RelE/RelB, ParE/ParD, HipA/HipB, PhD/Doc, Hok/Sok, YafM/YoeB, YafN/YafO, YgjM/YgjN, YgiT/YgiU, DinJ/YafQ, VapB/VapC, HipB/HipA, and HicB/HicA.

According to some embodiments of the invention, the anti-toxin is translationally fused to a destabilization sequence (e.g., the natural destabilization sequence of the antitoxin as present in the bacteria, e.g., in E. Coli).

MazF is a bacterial ribonuclease (e.g., SEQ ID NOs: 6 and 51), which is specific for ACA sequences in single-stranded RNA. MazF-induced toxicity is executed by blocking de novo protein synthesis through its endoribonuclease activity (mRNA interferases; Inouye et al., 2006). The MazE antitoxin (e.g., SEQ ID NOs: 13 and 52) interferes with the lethal action of the MazF toxin and neutralizes its toxicity.

According some embodiments of the invention, the toxin anti-toxin system comprise a MazEF system.

According some embodiments of the invention, the MazF toxin comprises the coding sequence set forth by SEQ ID NO:6.

According some embodiments of the invention, the MazF toxin protein is set forth by SEQ ID NO: 51.

According some embodiments of the invention, the MazE anti-toxin comprises the coding sequence set forth by SEQ ID NO:13.

According some embodiments of the invention, the MazE anti-toxin comprises the amino acid sequence set forth by SEQ ID NO: 52.

According some embodiments of the invention, the second promoter comprises CMV and the first promoter comprises SV40.

According some embodiments of the invention, the second nucleic acid sequence or the second nucleic acid construct further comprises a non-cancerous associated responsive element for regulating transcription of the anti-toxin.

Thus, by inclusion of the additional responsive element for regulating the transcription of the anti-toxin the construct or the construct system ensures that no-cell killing of “normal” (non-cancerous) cells.

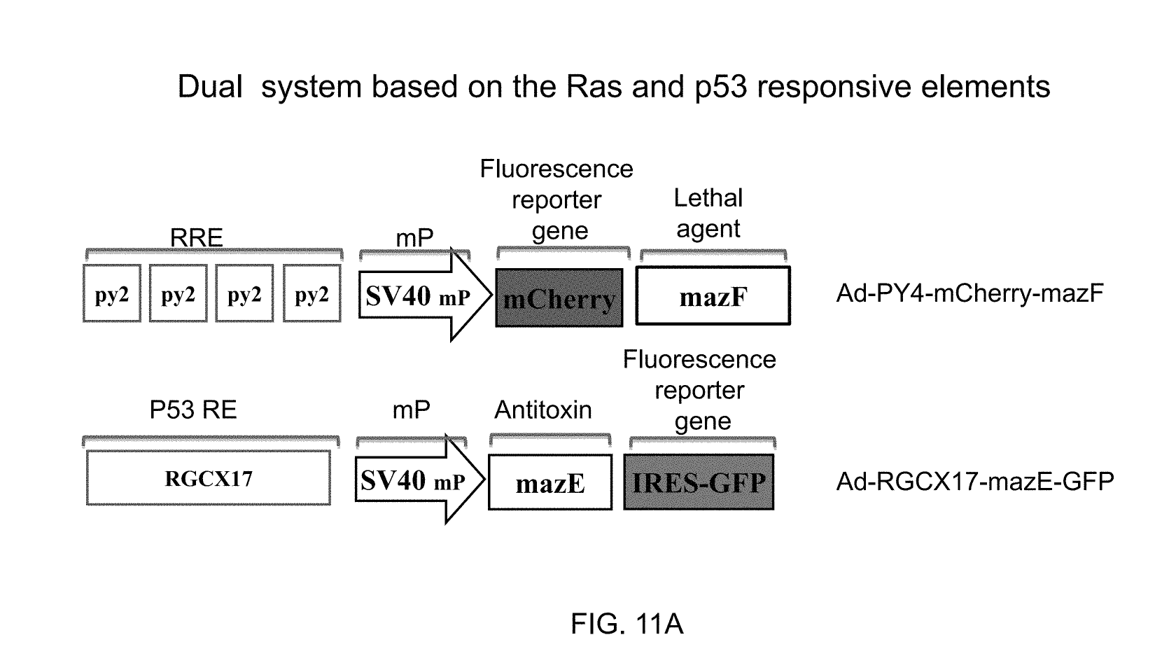

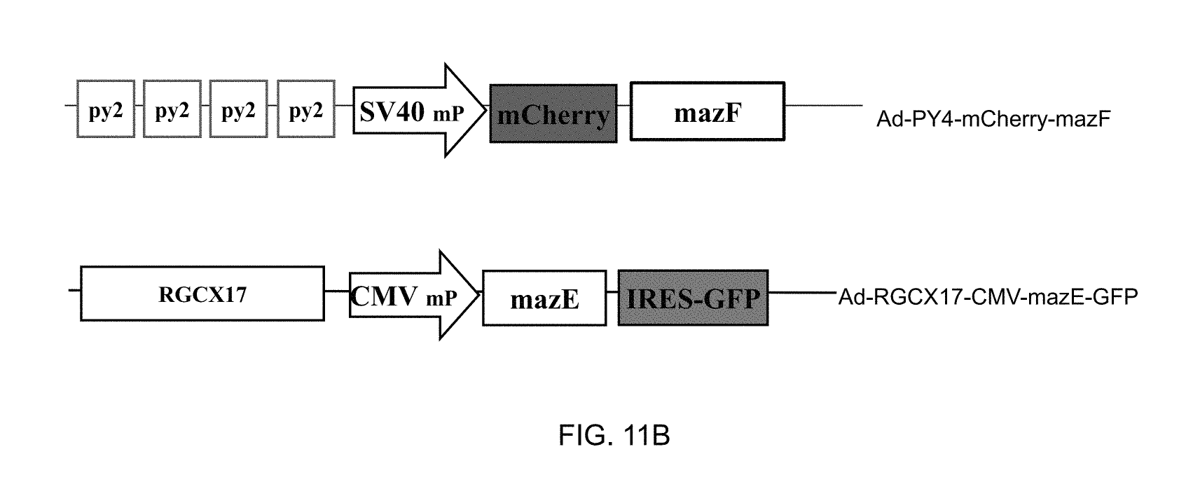

For example, as shown in FIGS. 11A and 11B the p53 wild type responsive element (such as provided in SEQ ID NO:14) can be added upstream of the promoter which drives the transcription of the antitoxin coding sequence.

According some embodiments of the invention, the non-cancerous associated responsive element comprises the p53 wild type responsive element.

According some embodiments of the invention, the p53 wild type responsive element is set forth by SEQ ID NO:14.

According some embodiments of the invention, the second nucleic acid sequence or the second nucleic acid construct comprises at least one copy of the p53 wild type responsive element set forth by SEQ ID NO:14.

According some embodiments of the invention, the second nucleic acid sequence or the second nucleic acid construct comprises at least 2 repeats of the non-cancerous associated responsive element, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 repeats of the non-cancerous associated responsive element.

According some embodiments of the invention, the second nucleic acid sequence or the second nucleic acid construct comprises 17 repeats of the p53 wild type responsive element.

According some embodiments of the invention, the second nucleic acid sequence or the second nucleic acid construct comprises the RCGX17 sequence (17 repeats of the p53 wild type responsive element) as set forth by SEQ ID NO: 15.

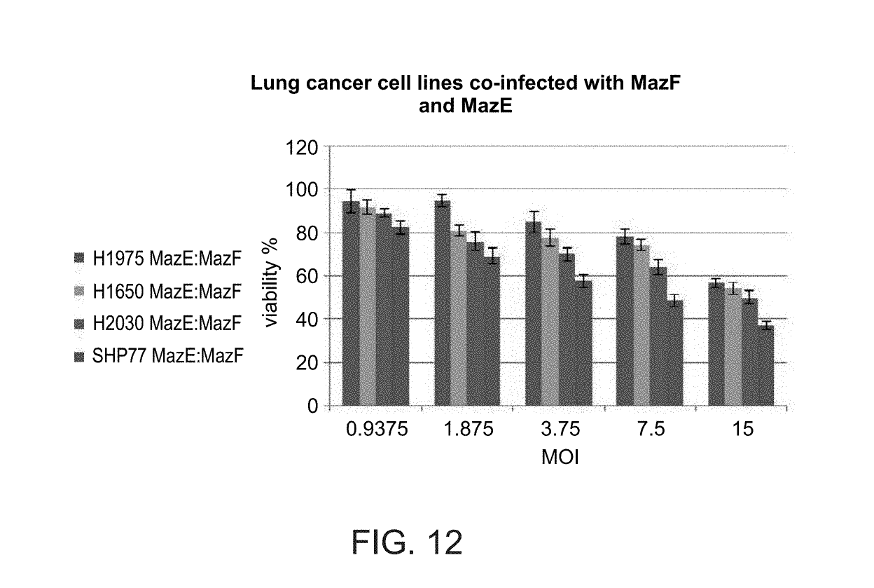

For example, as shown in FIG. 12, cancerous cells having a mutation in both ras and p53 (e.g., SHP77 cells which are Ras^{mut}/p53^{mut}) were more sensitive to cell killing than cells which exhibit wild type sequences of both ras and p53 (e.g., H1650 cells which are Ras^{wt}/p53^{wt}).

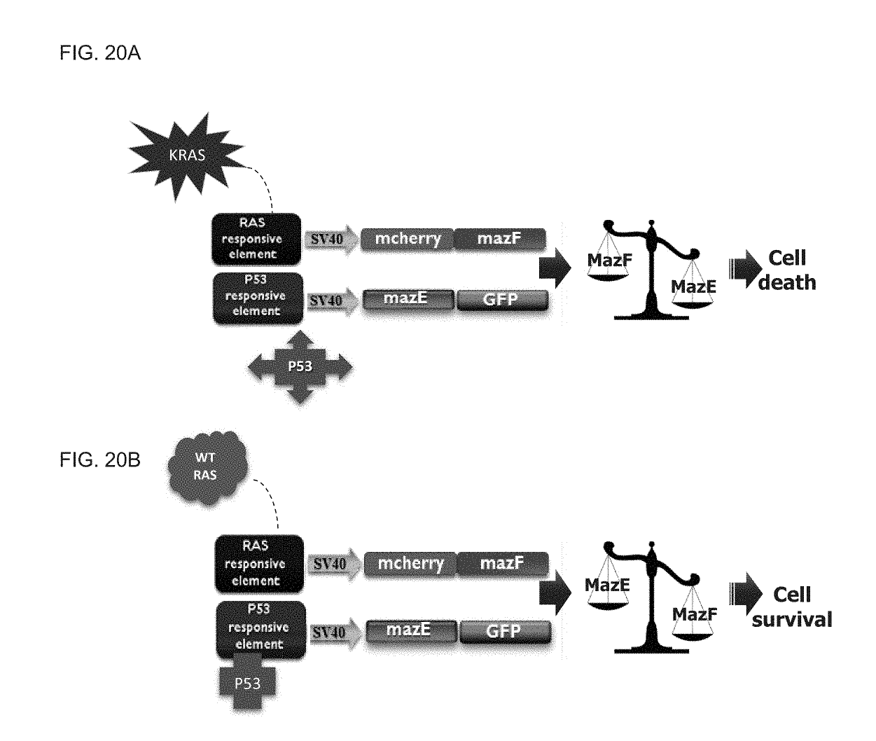

FIGS. 20A-B schematically describe a nucleic acid construct system according to some embodiments of the invention, which when interacting with a cancerous cell having a mutated K-Ras and mutated P53 the construct system leads to cell death (FIG. 20A) and when interacting with a non-cancerous cell having wild type (WT) Ras and wild type P53 the construct system leads to cell survival (FIG. 20B). It should be noted that in case the cancerous cell has a mutated Ras (e.g., K-Ras) and wild type P53 the interaction of such as cancerous cell with the nucleic acid construct system of some embodiments of the invention would still result in death of the cancerous cell (data not shown).

According to some embodiments of the invention, the first nucleic acid sequence or the first nucleic acid construct comprises four repeats of the PY2 sequence set forth by SEQ ID NO:2 being upstream and operably linked to the SV40 minimal promoter region set forth by SEQ ID NO:4, and a toxin coding sequence being downstream of and transcriptionally regulated by said SV40 minimal promoter region.

According to some embodiments of the invention, the second nucleic acid sequence or the second nucleic acid construct comprises the p53 wild type responsive element set forth by SEQ ID NO:14 being upstream and operably linked to the SV40 minimal promoter region set forth by SEQ ID NO:4, and an antitoxin coding sequence being downstream of and transcriptionally regulated by said SV40 minimal promoter region.

According to some embodiments of the invention, the second nucleic acid sequence or the second nucleic acid construct comprises about 17 repeats of said p53 wild type responsive element.

Additionally or alternatively, the nucleic acid construct or construct system can include a repressor-operator system for control of expression in cancerous cells having hyperactive cell signaling.

According some embodiments of the invention, the first nucleic acid sequence or the first nucleic acid construct further comprises a repressor of a bacterial repressor-operator system, the repressor being under a transcriptional regulation of the cancer-associated proliferative signaling responsive enhancer element, and wherein the second nucleic acid sequence or construct comprises an operator of the bacterial repressor-operator system, such that expression of the repressor inhibits expression of the antitoxin.

According some embodiments of the invention, the repressor comprises the Tetracycline repressor (Tet-R) sequence, and wherein the operator comprises the tetracycline operator sequence.

According some embodiments of the invention, the operator comprises at least two repeats of the sequence tetracycline operator sequence.

According some embodiments of the invention, the first nucleic acid sequence or the first nucleic acid construct comprises four repeats of the PY2 sequence set forth by SEQ ID NO:2 being upstream and operably linked to the SV40 minimal promoter region set forth by SEQ ID NO:4, a toxin coding sequence being downstream of and transcriptionally regulated by the SV40 minimal promoter region, an IRES sequence set forth by SEQ ID NO:7 being downstream and operably linked to the toxin coding sequence, and a Tetracycline repressor set forth by SEQ ID NO: 8 being downstream of and operably linked to the IRES sequence.

According some embodiments of the invention, the second nucleic acid sequence or the second nucleic acid construct comprises a CMV minimal promoter which comprises two repeats of a tetracycline operator as set forth by SEQ ID NO:9 and an antitoxin coding sequence being downstream of and operably linked to the CMV minimal promoter.

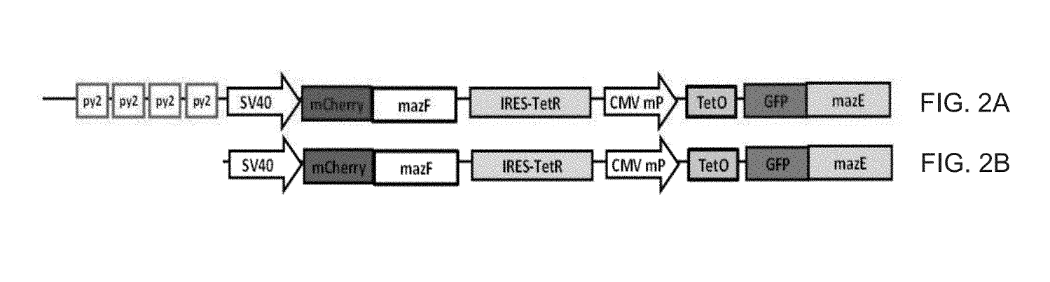

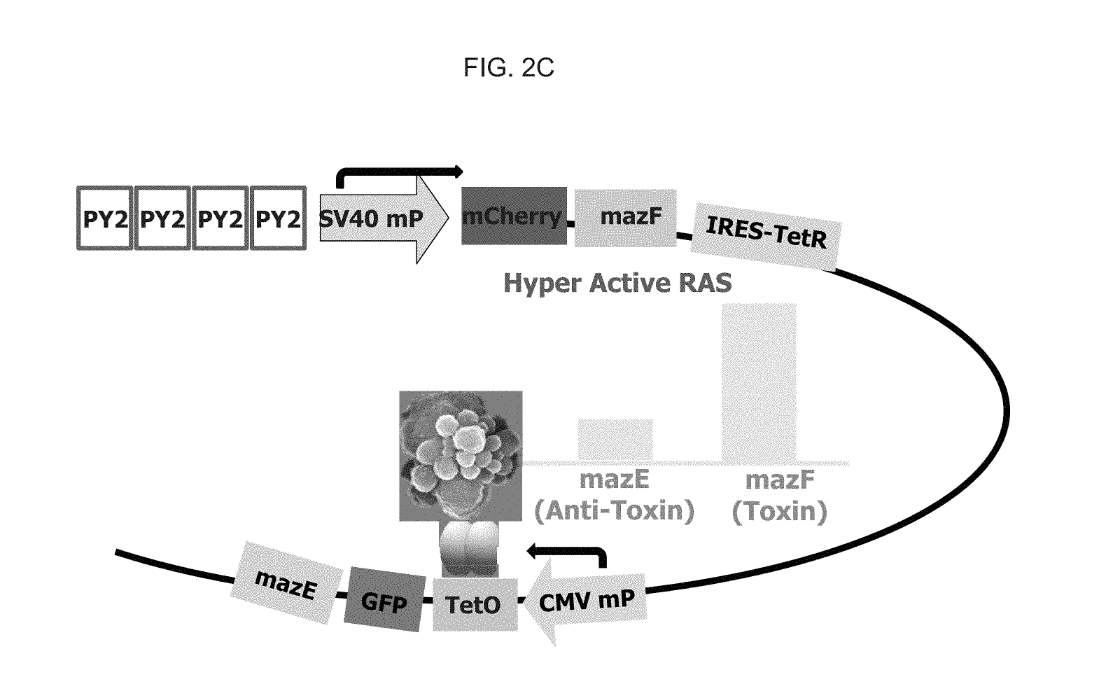

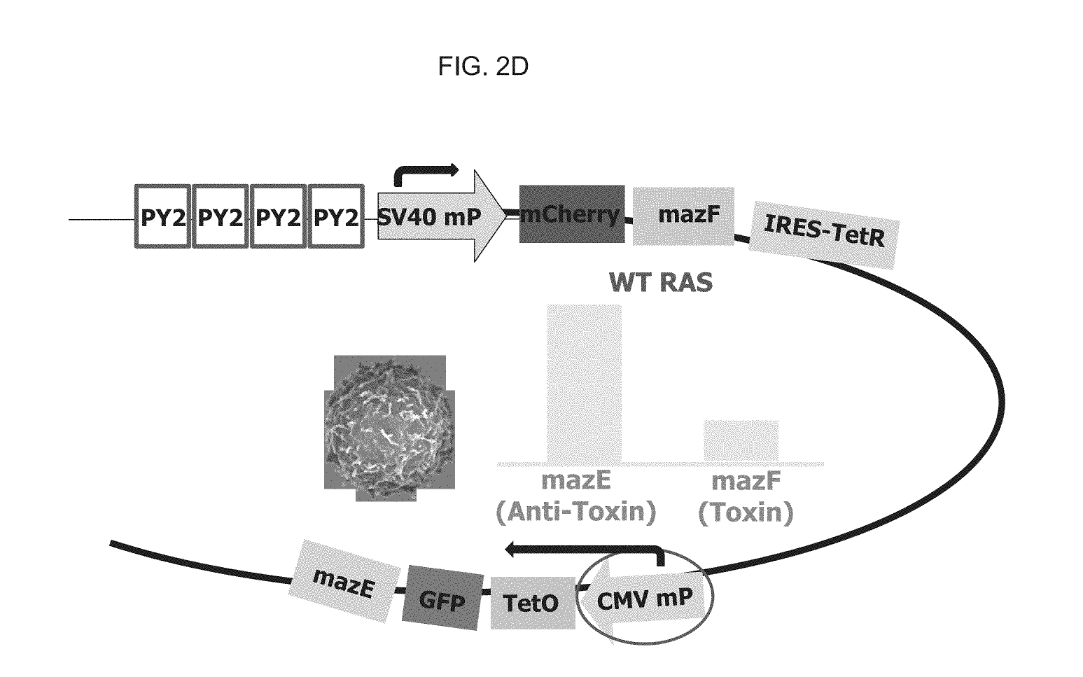

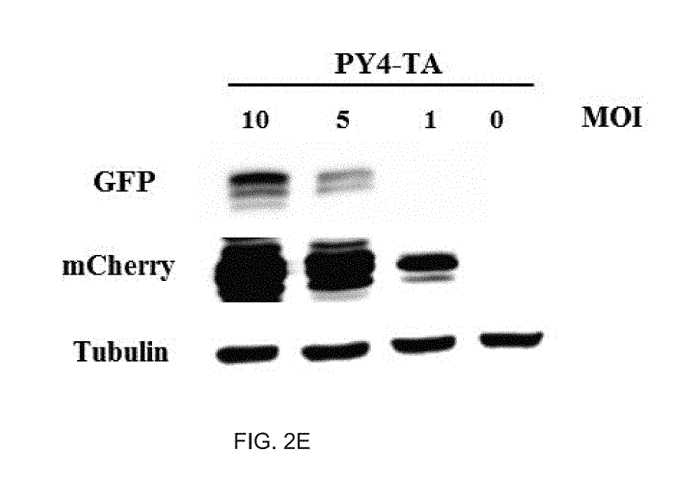

For example, as schematically illustrated in FIG. 2C, in cells with hyperactive RAS, the Tet-R is expressed resulting in its binding to the Tetracycline operator and accordingly inhibition of expression of the antitoxin mazE which is downstream to the Tetracycline operator. In contrast, in cells with wild type RAS the TetR is not overexpressed and accordingly does not bind to the tetracycline operator, resulting in expression of the antitoxin maze (FIG. 2D).

It should be noted that when the nucleic acid construct system is used, the first nucleic acid construct and the second nucleic acid constructs can be co-transfected at any MOI ratio into the cell-of-interest (e.g., the target cell such as the cancerous cell).

According to some embodiments of the invention, the first nucleic acid construct and the second nucleic acid constructs are co-transfected at a 1:1 MOI ratio.

For example, when using a nucleic acid construct system in which the second promoter is stronger than the first promoter, a 1:1 MOI ratio can be used for co-transfection.

Additionally or alternatively, when using a nucleic acid construct system in which the second promoter has a similar ability to direct transcription of a nucleic acid sequence operably linked thereto as the ability of the promoter of the first nucleic acid construct, and/or in the case of using identical promoters in both constructs (e.g., the SV40 promoter as shown in FIG. 11A) then the amount of the first nucleic acid construct should be higher than the amount of the second nucleic acid construct, in order to ensure cell killing.

According to some embodiments of the invention, the first nucleic acid construct and the nucleic acid construct are co-transfected into cells at an MOI ratio of 1 to 0.9, e.g., at an MOI ratio of 1 to 0.8, 1 to 0.7, 1 to 0.6, 1 to 0.5, 1 to 0.4, 1 to 0.3, 1 to 0.2, e.g., 1 to 0.1, respectively.

According to some embodiments of the invention, the first nucleic acid construct and the nucleic acid construct are co-transfected into cells at an MOI ratio of 1 to 0.5, respectively.

The nucleic acid construct of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of toxin or anti-toxin mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the nucleic acid construct of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The nucleic acid construct may or may not include a eukaryotic replicon. If a eukaryotic replicon is present, then the nucleic acid construct is amplifiable in eukaryotic cells using the appropriate selectable marker. If the nucleic acid construct does not comprise a eukaryotic replicon, no episomal amplification is possible. Instead, the recombinant DNA integrates into the genome of the engineered cell, where the promoter directs expression of the desired nucleic acid.

The nucleic acid construct of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding a toxin or anti-toxin can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

The cell-free particle of some embodiments of the invention can be administered to an organism per se, or in a pharmaceutical composition where it is mixed with suitable carriers or excipients.

According to an aspect of some embodiments of the invention, there is provided a pharmaceutical composition comprising the cell-free particle of some embodiments of the invention and a pharmaceutically acceptable carrier or diluents.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cell-free particle of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (the nucleic acid construct of some embodiments of the invention or the nucleic acid construct system of some embodiments of the invention) effective to prevent, alleviate or ameliorate symptoms of a disorder (e.g., cancer, e.g., colon cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to an aspect of some embodiments of the invention there is provided a method of treating cancer in a subject in need thereof. The method comprises administering to the subject a therapeutically effective amount of the pharmaceutical composition comprising the particles (as described herein), wherein the cancer cells of the subject are characterized by expression of a tumor marker to which the targeting moiety is directed, thereby treating cancer in the subject.

Methods of determining expression of a marker molecule include but are not limited to Western blot, Northern blot, reverse transcription-polymerase chain reaction (RT-PCR), quantitative PCR (Q-PCR), Enzyme-Linked Immunosorbent Assay (ELISA), immunohistochemistry staining, flow cytometry and fluorescence-activated cell sorter (FACS).

According to a specific embodiment, the cancer cells are characterized by hyper activity of the cancer-associated proliferative signaling as compared to non-cancerous cells of the same tissue.

Also provided is a composition comprising the cell-free particle for use in the treatment of cancer, wherein cancer cells of the cancer are characterized by expression of a tumor marker to which the targeting moiety is directed and optionally wherein the cancer cells are characterized by hyper activity of the cancer-associated proliferative signaling as compared to non-cancerous cells of the same tissue.

According to a specific embodiment, the tumor marker comprises CD24.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

Thus, the cell-free particle of some embodiments of the invention is administered to the cancer cells of a subject in need thereof.

As used herein, the term “subject” includes mammals, preferably human beings at any age which suffer from the pathology.

According to an aspect of some embodiments of the invention there is provided a composition comprising the cell-free particle of some embodiments of the invention for use in treating cancer, wherein cells of the cancer are characterized by hyper activity of the proliferative signaling as compared to non-cancerous cells of the same tissue.

According to some embodiments of the invention, the cancer comprises a solid tumor.

According to some embodiments of the invention, the cancer comprises cancer metastases and/or cancer micrometastases.

According to some embodiments of the invention, the cancer comprises cancer micrometastases.

Non-limiting examples of the cancer which can be treated by the composition (e.g., the nucleic acid construct or the nucleic acid construct system) or the method of some embodiments of the invention include any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to some embodiments of the invention, the cancer comprises colon cancer.

According to some embodiments of the invention, the cancer comprises lung cancer.

According to some embodiments of the invention, the cancer comprises pancreatic cancer.

According to some embodiments of the invention, the cancer comprises gastric cancer.

According to some embodiments of the invention, the cancer is characterized by a hyperactive RAS GTPase activity.

It should be noted that in cells where the RAS is hyperactive the hydrolysis of GTP (Guanosine-5′-triphosphate) to GDP (Guanosine diphosphate) is prevented, thus the ras pathway is always on (active) and is not deactivated, since it binds to GTP.

According to some embodiments of the invention, the RAS is a KRAS protein and wherein the hyperactive KRAS is caused by a G13D mutation in the KRAS protein set forth by SEQ ID NO:16 (i.e., substitution of glycine with aspartic acid at amino acid position 13 in the KRAS protein set forth by SEQ ID NO:16).

According to some embodiments of the invention, the RAS is an NRAS protein and wherein the hyperactive NRAS is caused by a Q61K mutation in the NRAS protein set forth by SEQ ID NO:17 (i.e., substitution of Glutamine with Lysine at amino acid position 61 in the NRAS protein set forth by SEQ ID NO:17).

According to some embodiments of the invention, the RAS is a HRAS protein and wherein the hyperactive HRAS is caused by a G12V mutation in the HRAS protein set forth by SEQ ID NO:18 (i.e., substitution of Glycine with Valine at amino acid position 12 of the HRAS protein set forth by SEQ ID NO:18).

According to some embodiments of the invention, the active agents described hereinabove (e.g., the nucleic acid construct of some embodiments of the invention and/or the nucleic acid construct system of some embodiments of the invention) can be provided to the subject in need thereof along with an additional medicament identified for treating the cancer, i.e., by combination therapy.

Therapeutic regimen for treatment of cancer suitable for combination with the nucleic acid construct of some embodiments of the invention and/or the nucleic acid construct system of some embodiments of the invention include, but are not limited to chemotherapy, biological therapy, immunological therapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

According to some embodiments of the invention, the method further comprising treating a subject having the cancer by a treatment selected from the group consisting of: chemotherapy, biological therapy, radiotherapy, phototherapy, photodynamic therapy, surgery, nutritional therapy, ablative therapy, combined radiotherapy and chemotherapy, brachiotherapy, proton beam therapy, immunotherapy, cellular therapy and photon beam radiosurgical therapy.

According to some embodiments of the invention, the composition (comprising the particles) further comprises an anti-cancer agent suitable for a treatment selected from the group consisting of: chemotherapy, biological therapy, photodynamic therapy, nutritional therapy, brachiotherapy, immunotherapy, and cellular therapy.

According to a specific embodiment, the anti-cancer agent is encapsulated in or conjugated to the particle.

It should be noted that such synergistic activity of treatment with the cell-free particle of some embodiments of the invention with additional therapeutic methods or compositions has the potential to significantly reduce the effective clinical doses of such treatments, thereby reducing the often devastating negative side effects and high cost of the treatment.

Anti-cancer drugs that can be co-administered with the compounds of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Approved chemotherapy include, but are not limited to, abarelix, aldesleukin, aldesleukin, alemtuzumab, alitretinoin, allopurinol, altretamine, amifostine, anastrozole, arsenic trioxide, asparaginase, azacitidine, bevacuzimab, bexarotene, bleomycin, bortezomib, busulfan, calusterone, capecitabine, carboplatin, carmustine, celecoxib, cetuximab, cisplatin, cladribine, clofarabine, cyclophosphamide, cytarabine, dacarbazine, dactinomycin, actinomycin D, Darbepoetin alfa, Darbepoetin alfa, daunorubicin liposomal, daunorubicin, decitabine, Denileukin diftitox, dexrazoxane, dexrazoxane, docetaxel, doxorubicin, dromostanolone propionate, Elliott's B Solution, epirubicin, Epoetin alfa, erlotinib, estramustine, etoposide, exemestane, Filgrastim, floxuridine, fludarabine, fluorouracil 5-FU, fulvestrant, gefitinib, gemcitabine, gemtuzumab ozogamicin, goserelin acetate, histrelin acetate, hydroxyurea, Ibritumomab Tiuxetan, idarubicin, ifosfamide, imatinib mesylate, interferon alfa 2a, Interferon alfa-2b, irinotecan, lenalidomide, letrozole, leucovorin, Leuprolide Acetate, levamisole, lomustine, CCNU, meclorethamine, nitrogen mustard, megestrol acetate, melphalan, L-PAM, mercaptopurine 6-MP, mesna, methotrexate, mitomycin C, mitotane, mitoxantrone, nandrolone phenpropionate, nelarabine, Nofetumomab, Oprelvekin, Oprelvekin, oxaliplatin, paclitaxel, palifermin, pamidronate, pegademase, pegaspargase, Pegfilgrastim, pemetrexed disodium, pentostatin, pipobroman, plicamycin mithramycin, porfimer sodium, procarbazine, quinacrine, Rasburicase, Rituximab, sargramostim, sorafenib, streptozocin, sunitinib maleate, tamoxifen, temozolomide, teniposide VM-26, testolactone, thioguanine 6-TG, thiotepa, thiotepa, topotecan, toremifene, Tositumomab, Trastuzumab, tretinoin ATRA, Uracil Mustard, valrubicin, vinblastine, vinorelbine, zoledronate and zoledronic acid.

Anti-cancer biological drugs that can be co-administered with the compounds of the invention include, but are not limited to bevacizumab (AVASTIN™ Genentech Inc.), Cetuximab (ERBITUX™ ImClone Systems Incorporated), Panitumumab (VECTIBIX™ Immunex Corporation) and/or any combination thereof.

Additional anti-cancer drugs that can be co-administered with the compounds of the invention include, but are not limited to 5-FU, Capecitabine (XELODA™ Hoffmann-La Roche, Inc), Irinotecan (CAMPTOSAR™ YAKULT HONSHA COMPANY, LTD), Oxaliplatin (ELOXATIN™ Sanofi), Trifluridine and tipiracil (LONSURF™ TAIHO PHARMACEUTICAL CO., LTD.), Gemcitabine (GEMZAR™ Eli Lilly and Company), Albumin-bound paclitaxel (ABRAXANE™ of ABRAXIS BIOSCIENCE, LLC), Cisplatin, Paclitaxel (TAXOL™ Bristol-Myers Squibb Company), Docetaxel (TAXOTERE™ AVENTIS PHARMA S.A.), Irinotecan liposome (ONIVYDE™ Merrimack Pharmaceuticals, Inc.), dacarbazine (DTIC-DOME™ BAYER HEALTHCARE PHARMACEUTICALS INC.), ETOPOSIDE (ETOPOPHOS™ Bristol-Myers Squibb Company), Temozolomide (TEMODAL™ Schering Corporation), lapatinib (Tyverb™ GlaxoSmithKline), erlotinib (Tarceva™ Astellas Pharma Inc.), everolimus (AFINITOR™ Novartis AG), and/or any combination thereof. It should be noted that any combination of known anti-cancer treatment (e.g., biological, immunological, chemotherapy and the like) can be combined with the gene therapy approach of the claimed compositions

The disclosure also encompasses a packaged and labelled pharmaceutical product. This article of manufacture or kit includes the appropriate unit dosage form in an appropriate vessel or container such as a glass vial or plastic ampule or other container that is hermetically sealed. Preferably, the article of manufacture or kit further comprises instructions on how to use including how to administer the pharmaceutical product. The instructions may further contain informational material that advises a medical practitioner, technician or subject on how to appropriately prevent or treat the disease or disorder in question. In other words, the article of manufacture includes instructions indicating or suggesting a dosing regimen for use including but not limited to actual doses, monitoring procedures, and other.

As with any pharmaceutical product, the packaging material and container are designed to protect the stability of the product during storage and shipment.

The kits may include the cell-free particle of some embodiments of the invention (e.g., the exosome(s)) in sterile aqueous suspensions that may be used directly or may be diluted with normal saline for intravenous injection or use in a nebulizer, or dilution or combination with surfactant for intratracheal administration. The kits may therefore also contain the diluent solution or agent, such as saline or surfactant. The kit may also include a pulmonary delivery device such as a nebulizer or disposable components therefore such as the mouthpiece, nosepiece, or mask.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO:6 is expressed in a DNA sequence format (e.g., reciting T for thymine), but it can refer to either a DNA sequence that corresponds to an E. coli MazF ribonuclease nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Reagents—

All reagents were purchased from Sigma, Israel unless otherwise stated. All secondary HRP-conjugated antibodies were from Jackson ImmunoResearch Laboratories, USA. ECL reagent, cell culture media and additives were from Beit-Haemek, Israel. Nitrocellulose filters were from Schleicher & Schuell BioScience, USA. Annexin V and Reddot2 dye were purchased from Biotium, and G418 was purchased from Gibco. All plasmid and DNA fragment purifications were carried out with a High-Speed Plasmid Mini Kit and a Zymoclean™ Gel/PCR DNA recovery Kit (Fermentas and Zemo Research, respectively) unless otherwise specified. T4 DNA ligase and restriction enzymes were purchased from New England Biolabs, USA. DNA ligations were carried out overnight at 16° C.

Bacterial Strains—

The following Escherichia coli (E. coli) strains were used: DH5a (Stratagene, USA) for plasmid propagation and BJ5183 (Stratagene, USA) for the generation of recombinant adenovirus plasmid DNA.

Cell Lines—

HT29 human colon adenocarcinoma, HCT116 human colon cancer, R1 KRAS transformed rat enterocytes and HEK293 human kidney cell lines were grown in high-glucose Dulbecco's modified Eagle's medium (DMEM), all supplemented with 5% heat-inactivated fetal bovine serum (FBS), 1% penicillin and streptomycin in an atmosphere of 95% oxygen and 5% CO₂. 1 μg/ml tetracycline was added to the HEK293 medium for TA virus production. In addition, 600 μg/ml G418 was added to the culture medium of MazE-expressing cells.

Oligonucleotides—

All the oligonucleotides that were used in this study were purchased from Sigma, Israel.

Recombinant DNA techniques were carried out according to standard protocols or as recommended by the manufacturers. A more detailed description of the procedure is provided hereinbelow.

Construction and Propagation of Recombinant Adenovirus Vectors

Construction of the Vector Encoding for “mCherry-MazF”—

The monomeric red fluorescence protein mCherry was amplified from an expression cassette by PCR.

The PCR product was digested with HindIII and XbaI and cloned between the corresponding sites of the plasmid “pGL3 promoter-Py4-PUMA” (replacing the PUMA gene) that had been previously prepared, generating the “pGL13 promoter-Py4-mCherry” plasmid.

The MazF coding sequence which was amplified from an expression cassette (kindly provided by Dr. Assaf Shapira, Department of Molecular Microbiology and Biotechnology, Tel-Aviv University, Israel) using the primes Hind-Cher-For and Xba-Maz-Rev [5′-CTTTTGCAAAAAGCTTCCACCATGggaattcacGTGAGCAAGGGCGAGGAGG-3′ (SEQ ID NO:21) and 5′-CCGCCCCGACTCTAGActaaccggtgccaatcagtacgttaattttggc-3′ (SEQ ID NO:22), respectively] and was fused to the C terminus of the mCherry under the RRE, Py4-SV40 mP, generating the “pGL3 promoter-Py4-mCherry-MazF” plasmid. This intermediate vector was used as the template for the amplification of the Py4-mCherry-MazF fusion genes, and the amplified product was cloned using the AdEasy system (pShuttle and pAdEasy-1), as previously described (He et al., 1998, Luo et al., 2007) to generate the regulated expression cassette “pAdEasy-Py4-mCherry-MazF”.

Construction of the Vector Encoding for “mCherry”—

The sequence of the red fluorescent protein mCherry3 was amplified by PCR from an expression cassette and cloned downstream to a CMV promoter, generating the expression cassette “pAdEasy-CMV-mCherry”.

Construction of the Vector Encoding for “MazEF”—

The tetracycline repressor coding sequence was located downstream to an IRES sequence and cloned downstream to the SV40-mCherry-MazF sequence. An additional arm was introduced to this cassette, controlled by a different minimal promoter (mP) without the RAS-responsive elements. Starting from its N terminus, this arm includes: a CMV mP, a tetracycline operator sequence, the full MazE coding sequence, IRES and EGFP coding sequence (FIG. 2A), generating the expression cassette “pAdEasy-Py4-mCherry-MazF-IRES-TetR-CMVmp-MazE-IRES-EGFP”.

Plasmids were isolated by a standard miniprep procedure and sequenced to confirm their predicted composition. For the production of virus particles, the plasmids “pAdEasy-Py4-mCherry-MazF”, “pAdEasy-Py4-mCherry-MazF-IRES-TetR-CMVmp-MazE-IRES-EGFP”, “pAdEasy-SV40-mCherry-MazF-IRES-TetR-CMVmp-MazE-IRES-EGFP” and “pAdEasy-mCherry” were isolated from selected “positive” clones and digested with Pad. Next, DNA was purified using a Zymoclean™ Gel DNA Recovery Kit according to the manufacturer's instructions. Five micrograms (μg) of the purified, digested plasmids were used to transfect 70% confluence HEK293 cells (supplemented with tetracycline at a final concentration of 1 μg/ml when the TA construct had been transfected) and HEK293-MazE cells (for the mCherry-MazF construct, supplement S2) in 60-mm culture plates using the calcium-phosphate method. After 24 hours, the transfection medium was replaced with 5 ml of fresh medium (that was supplemented with 1 μg/ml of tetracycline in the TA system). From 7 to 10 days post-transfection, when a cytopathic effect (CPE) had been clearly observed, the cells were collected by scraping them off the plate and pelleting them along with any floating cells in the culture. The pellet was washed once with phosphate-buffered saline (PBS), suspended in 0.5 ml PBS and subjected to 4 cycles of freeze/thaw. Cell debris was precipitated by brief centrifugation, and 300 μl of the supernatant that contained virus particles were used to infect 70% confluent HEK293 and its derivative cells in 10 cm plates (first amplification “cycle”). When one-third to one-half of the cells had been detached (usually after 3-5 days), virus particles were released by freeze/thaw cycles as described above. The supernatant containing viruses was kept at −80° C.

High scale production of the adenoviruses was performed by SIRION biotech, Germany.

Establishment of New Packaging Cell Line for the Production of the Toxin—

The lethal transgene MazF coding sequence was fused to the mCherry gene and cloned into the Ad5 adenoviral vector plasmid DNA. The propagation of the virus particles in the HEK293 packaging cells resulted in low yields of virus production due to the highly toxic nature of the selected gene. Hence, an innovative packaging system was established, based on MazF-resistant HEK293 cells that constitutively express the MazE antitoxin, encoded from the pIRES2-MazE-IRES-EGFP plasmid. The EGFP marker was used for effective and rapid screening of stable clones according to their high fluorescence intensity. The yield was higher by almost 2 orders of magnitude after propagation in MazF-resistant cells compared to propagation in parental HEK293 packaging cells.

Cell-Viability Assay—

The cell-killing activities of adenoviruses encoding for MazF, and MazEF were measured by the Thiazolyl Blue Tetrazoliam Bromide (MTT) enzymatic assay. Briefly, 1×10⁴ cells were seeded in 96-well plates. After 24 hours, different dilutions of recombinant adenoviruses encoding for the above-described cassettes were added. At 72 hours post infection, the media was replaced by fresh media (100 μl per well) containing 1 mg/ml MTT and the cells were incubated for 2-4 more hours. MTT-formazan crystals were dissolved by the addition of extraction solution (0.1N HCl in absolute isopropanol). Absorbance at 570 nm and a reference wavelength of 690 nm was recorded on an automated microplate reader.

Detection of Cell Death

Apoptosis—

Cells were seeded in 12-well plates (1×10⁵ cells/well) in complete medium and infected with the different adenoviruses at several multiple of infection (MOI) for 72 hours. Annexin V (Annexin V, CF640R conjugate) was detected according to the manufacturer's protocol (Biotium Inc., USA). The cells were washed with PBS and then incubated in a solution of Annexin V binding protein. The cells were analyzed by flow cytometry [FACSCalibur (Becton Dickinson, CA)], and the results were analyzed with the CELLQuest program (Becton Dickinson).

Total Dead Cells—

Cells were seeded in 12-well plates (1×10⁵ cells/well) in complete medium and infected with the different adenoviruses at several MOI for 72 hours. Dead cells were detected by RedDot™2, a far-red cell membrane-impermeable nuclear dye, according to the manufacturer's protocol (Biotium Inc., USA). The cells were washed with PBS, and then incubated in a solution of RedDot2 dye. Far red nuclear staining was detected by flow cytometry.

End-Point Dilution Assay (EPDA)—

1×10⁴ HEK293 cells/well were seeded in 96-well plate in 100 μl of growth medium. The recombinant adenovirus stock solutions were serially diluted 10-fold to a concentration in a range of 10⁻³-10⁻¹⁰ into growth medium and added to each well in columns 1-10. Virus-free growth medium was added to the wells in columns 11 and 12 which served as controls for the viability of non-infected cells. The plate was incubated in a humidified CO₂ (5%) incubator for 10 days at 37° C. Each well was checked for CPE using a microscope. A well was scored as CPE positive even if only a few cells showed cytopathic effects. The viral titer was calculated according to the formula: Titer (pfu/ml)=10^{(x+0.8)}, where x=the sum of the fractions of CPE-positive wells for each dilution (10 out of 10 wells with CPE calculated as “1”).

Colony Formation Assay—

5×10⁵ HCT116 and HT29 cells were seeded per well in 6-well plates. After 24 hours, the cells were infected with 25 and 10 MOI of the viruses “pAdEasy-Py4-mCherry-MazF-IRES-TetR-CMVmp-MazE-IRES-EGFP” and “pAdEasy-SV40-mCherry-MazF-IRES-TetR-CMVmp-MazE-IRES-EGFP” or left un-infected. After 7 hours, the cells were trypsinized and seeded in 3-fold dilutions and incubated for 7 days. Surviving colonies were fixed with 4% formaldehyde in PBS and stained with 0.02% crystal violet.

Xenograft Model in Mice for Measuring In Vivo Tumor Development—

Male 6-8 week old athymic nude mice (Harlan Laboratories) (n=18) were housed in sterile cages and handled with aseptic precautions. The mice were fed ad libitum. For testing the therapeutic potential of the TA system, exponentially growing HCT116 cells were harvested and resuspended at a final concentration of 5×10⁶ cells per 0.1 ml PBS per injection. The cells were injected subcutaneously at two sites on the backs of the mice. When tumors were palpable (˜0.3-0.5 cm³), the mice were randomly divided into three groups of six each and the treatment was started. The viruses Ad-Py4-TA (6 mice) and ΔPy4-TA 1×10⁹ pfu (6 mice) or PBS (6 mice) were administrated via two intraperitoneal injections with a 3-day interval between injections. The mice were weighed, the tumor volume was measured with a caliper every two days starting from treatment onset and those results were carefully plotted. Tumor volume was calculated as 4/3π·a·b². At the end of the experiment, MazF and MazE expression in the tumors was monitored by imaging using the CRi Maestro system. The mice were anesthetized and then sacrificed by cervical dislocation and the tumors were excised.

TABLE 3
Table 3.
RT-PCR primers

| | | SEQ | | SEQ |
|---|---|---|---|---|
| | Forward primer | ID | Reverse primer | ID |
| Gene | (5′-3′) | NO: | (5′-3′) | NO: |
| P53 | CCCAAGCAATGGATG | 25 | GGCATTCTGGGAGCTTCA | 36 |
| | ATTTGA | | TCT | |
| P21 | GGCAGACCAGCATGA | 26 | GCGGATTAGGGCTTCCTC | 37 |
| | CAGATT | | TT | |
| Bax | TGAGCAGATCATGAA | 27 | GCTCGATCCTGGATGAA | 38 |
| | GACAGGG | | ACC | |
| Noxa | AGAGCTGGAAGTCGA | 28 | GCACCTTCACATTCCTCT | 39 |
| | GTGT | | C | |
| Puma | TCAACGCACAGTACG | 29 | GTAAGGGCAGGAGTCCC | 40 |
| | AGCG | | ATG | |
| MDM2 | CAGGCAAATGTGCAA | 30 | GGTTACAGCACCATCAGT | 41 |
| | TACCAA | | AGGTACAG | |
| 14-3-3 | GCCTATAAGAACGTG | 31 | CCTCGTTGCTTTTCTGCT | 42 |
| | GTGGGC | | CAA | |
| CD95 | CCCTCCTACCTCTGGT | 32 | TTGAATGTCAGTCACTTG | 43 |
| | TCTTACG | | GGCAT | |
| Btg2 | CCAGGAGGCACTCAC | 33 | GCCCTTGGACGGCTTTTC | 44 |
| | AGAGC | | | |
| GADD45 | CTCAACGTCGACCCC | 34 | ACATCTCTGTCGTCGTCC | 45 |
| | GATAA | | TCG | |
| Survivin | CCACCGCATCTCTAC | 35 | CAAGTCTGGCTCGTTCTC | 46 |
| | ATTCA | | AGT | |

Statistics—

Data from the in vitro studies are presented as mean±SD (standard deviation) of sets of data as determined in triplicates. Statistical significance between treatments was determined by Student t-test, P values <0.05 were considered significant.

In the in vivo studies, the tumor-bearing mice were randomized into various treatment groups (n=6) and the tumor volumes were periodically monitored and calculated as 4/3π·a·b². Statistical significant differences between groups and at different time points were determined by Student t-test.

Study Approval—

The study was approved by the Institutional committee for animal welfare at Tel-Aviv Sourasky Medical Center.

### Example 1

Experimental Results

The Activity of the PY4 Ras-Responsive Element was Tested in HCT116 Cells—

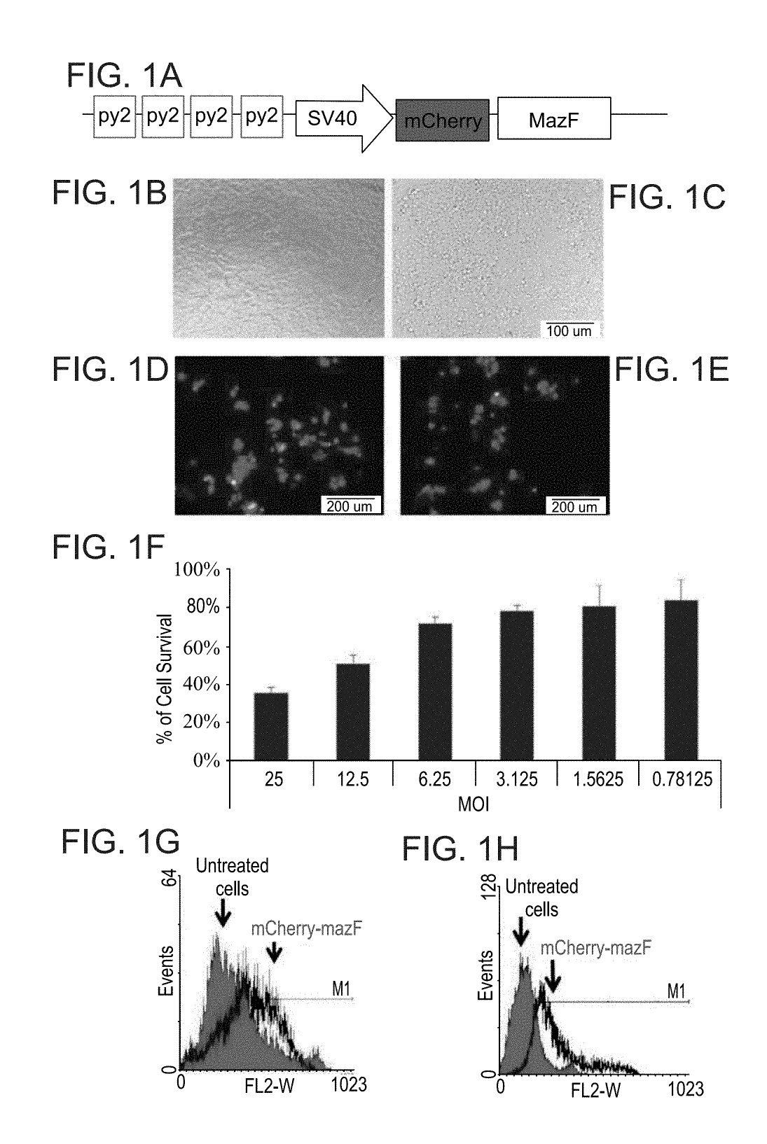

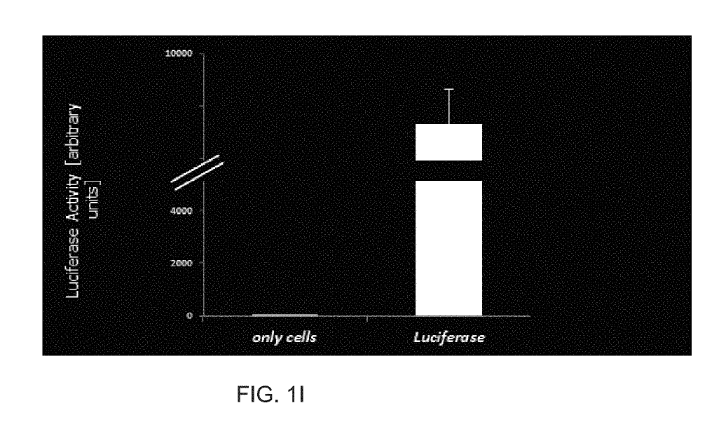

The activity of the KRAS pathway was evaluated in mutated CRC cells (HCT116). HCT116 cells were transfected with the luciferase vector (and Renilla plasmid) in which luciferase expression is under the control of the SV40 promoter and the PY4 enhancer (the luciferase construct is depicted in FIG. 9B). HCT116 CRC cell line is highly responsive to the Ras-activated promoter containing the Py2 Repeats (PY4). The Luciferase activity was normalized to Renilla Luciferase activity from a parallel co-transfection (FIG. 1I). As shown in FIG. 1I, the luciferase activity was significantly higher in the transfected cells compared to the untransfected cells.

Eradication of Mutated RAS-Harboring Cells by Adenovirus-Mediated Delivery of MazF Ribonuclease—

The potency and ability of MazF to kill the target cells were evaluated prior to engineering a more complex system with several toxicity control points. Massive cell death, in a dose-dependent manner, was induced following infection of HCT116 cells [containing a mutated KRAS at codon 13 (Gly to Asp)] with Ad-Py4-SV40-mCherry-MazF (FIG. 1A). FIGS. 1A-H show the cytotoxicity induced by the ribonuclease activity qualitatively evaluated by a fluorescent microscope examination 72 hours after the infection (FIGS. 1D-E) as compared to the uninfected cells (FIGS. 1B-C). About 35% cell survival (relative to the uninfected controls) was quantitatively measured by the enzymatic MTT assay upon treatment, when employing a MOI of 25 (FIG. 1F). Cytotoxic activity of MazF was confirmed by FACS analysis: 50% apoptosis was measured using annexin V (FIG. 1G), while about 80% membrane compromised or dead cells was detected with RedDot2 (FIG. 1H).

### Example 2

### Design of a Toxin-Antitoxin Cassette Utilizing Hyperactive RAS in Cancer Cells

Rational Design of an Innovative Toxin-Antitoxin Cassette for Enhanced Regulation—

The rationale behind the design of the“pAdEasy-Py4-SV40Mp-mCherry-MazF-IRES-TetR-CMVmp-MazE-IRES-EGFP” construct (FIG. 2A), or briefly “pAdEasy-Py4-TA”, was to couple the ribonuclease activity with its antidote in order to enable protection of non-target cells (i.e., normal cells without a hyperactive RAS pathway) while allowing a high level of expression of the toxic agent in mutated RAS-harboring cells.

In hyperactive RAS cells (FIG. 2C), the Py4 enhancer element induced toxin expression significantly more than that of the antitoxin. The Tet repressor, which is also expressed in high levels, binds to the Tet operator sequence and further inhibits the transcription of the antitoxin. Altogether, MazF is expected to overcome the antitoxin inhibition and the cells should die.

In cells that do not harbor mutated RAS (FIG. 2D), the Py4 enhancer is not activated, therefore there is no preference for expression from the SV40 mP. Since the CMV mP is slightly stronger than the SV40 mP and one molecule of the AT inhibits two molecules of toxin, the inhibitory activity of the antitoxin should prevail.

Consequently, the MazE in these cells will overcome the toxicity of MazF and the cells will survive. FIG. 2E shows a representative Western blot that confirms the differences in the degree of expression of the toxin (represented by the mCherry) vs. antitoxin (represented by the GFP). In cells with mutated KRAS, the expression of the toxin is higher than the antitoxin upon infection with the PY4-TA viruses.

### Example 3

### Maze Protects Normal Cells from MazF Cytotoxic Activity

MazE Protects Normal Cells from MazF Cytotoxic Activity

Experimental Results

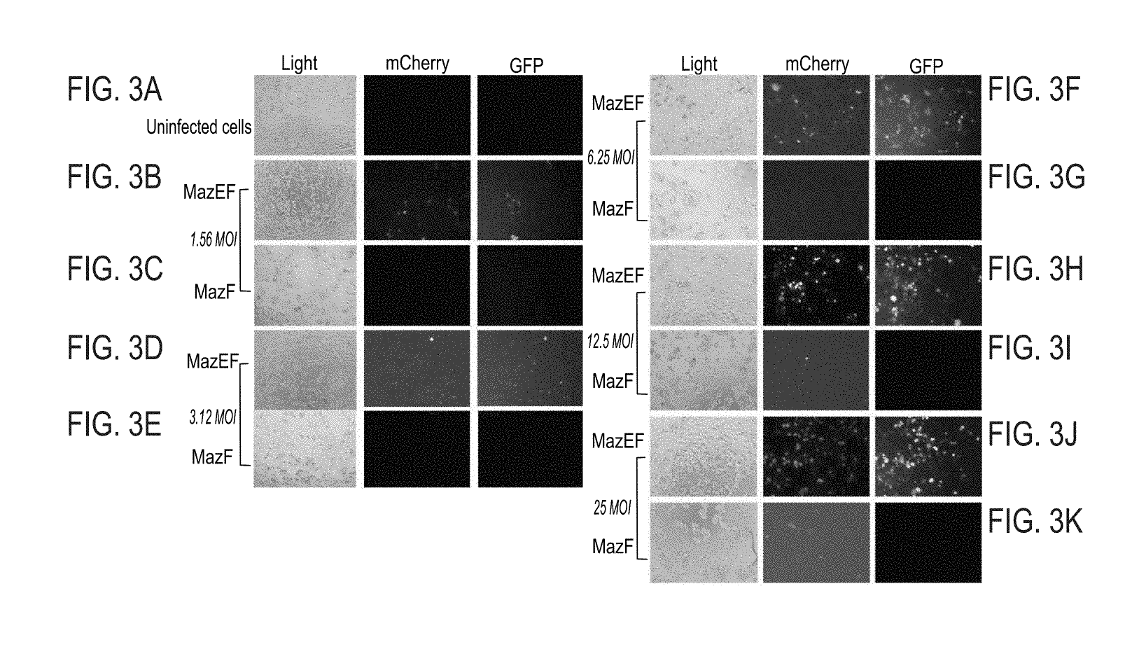

In order to demonstrate the advantage of using the pAdEasy-Py4-TA cassette, the present inventors tested its ability to protect cells with wild type (WT) RAS from possible “leakage” of the lethal gene. The basal expression from the SV40 mP along with low expression levels of RAS in normal cells induce low expression of MazF. However, even this low level of expression is sufficient to kill a cell. HT29 cells, with WT RAS, were infected with twofold dilutions of the MazEF- or MazF-encoding viruses, and the viability of the cells was qualitatively examined by light and fluorescence microscopy. As shown in FIGS. 3C, 3E, 3G, 3I, and 3K, infection with MazF decreased cell viability, indicating a leakiness of MazF expression even in the absence of mutated RAS. In contrast, infection with the MazEF construct was well tolerated. When visualized under a fluorescence microscope, the intoxicated Ad-Py4-SV40-mCherry-MazF-infected cells showed very faint red fluorescence, indicating inefficient mCherry-MazF accumulation. This is due to the ribonuclease activity of MazF that results in inhibition of protein synthesis, including its own (Zhang 2003; Shapira et al., 2012). On the other hand, the ribonuclease activity of MazF was neutralized by its antidote MazE in cells infected with pAdEasy-Py4-TA, as indicated by the presence of both red and green fluorescence (FIGS. 3B, 3D, 3F, 3H and 3J).

### Example 4

### The MazEF Cassette is Capable of Killing Cells Harboring Hyperactive RAS

Experimental Results

Adenovirus-Mediated Delivery of TA-Encoding Cassette Specifically Eliminates R1 Cells Harboring Activated RAS—

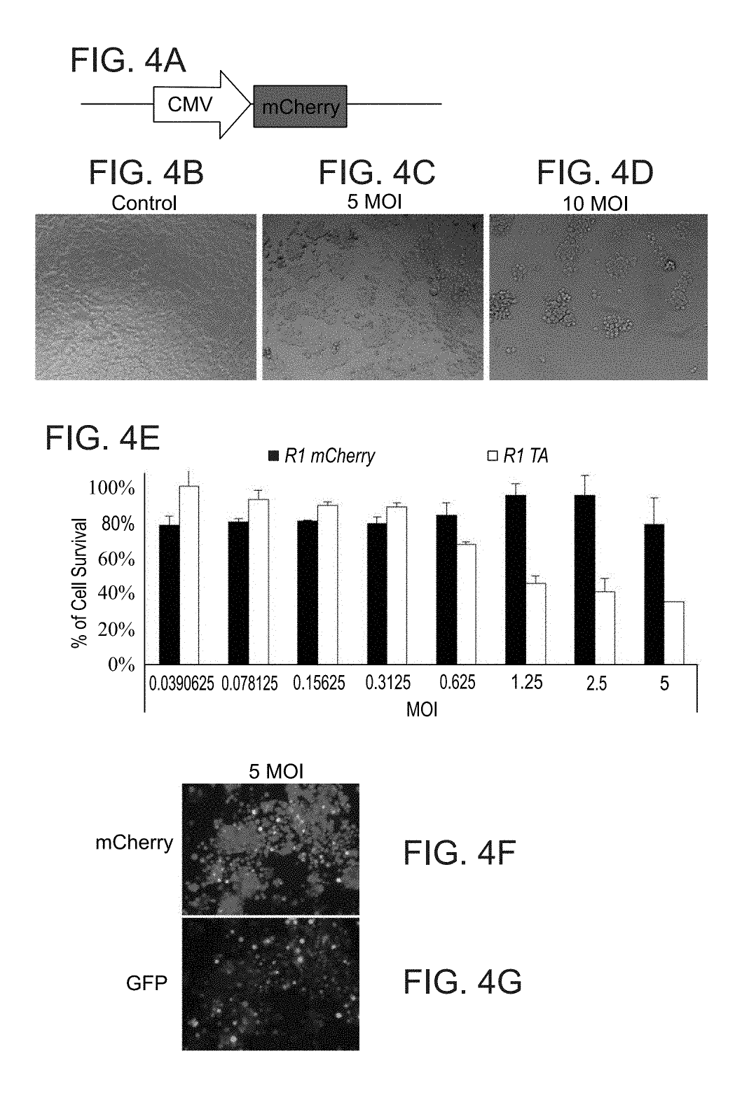

The potential of the MazEF-encoding cassette to kill target cells was tested in a proof of concept study performed in R1 cells, which serve as a model system for hyperactive RAS-harboring cells (Arber et al., 1996). R1 cells were infected with twofold dilutions of the mCherry encoding virus (the mCherry construct is schematically depicted in FIG. 4A) or of the PY4-TA (MazEF-encoding) virus (FIG. 2A). Infection of R1 cells with MazEF elicited a considerable cytotoxic effect, decreasing viability to 36% (at MOI 5) relative to the uninfected controls, while no significant effect was seen after infection with the mCherry cassette (80-90%) (shown qualitatively by light microscope in FIGS. 4B-D and quantitatively by MTT in FIG. 4E). The expression of the GFP and mCherry proteins indicates that both MazF and MazE components had been expressed (FIGS. 4F-G). However, although both, mazF and mazE were expressed, the mazF overcame the inhibition of the antitoxin in KRAS mutated cells;

The MazEF Cassette Kills Mutated RAS-Harboring Cells—

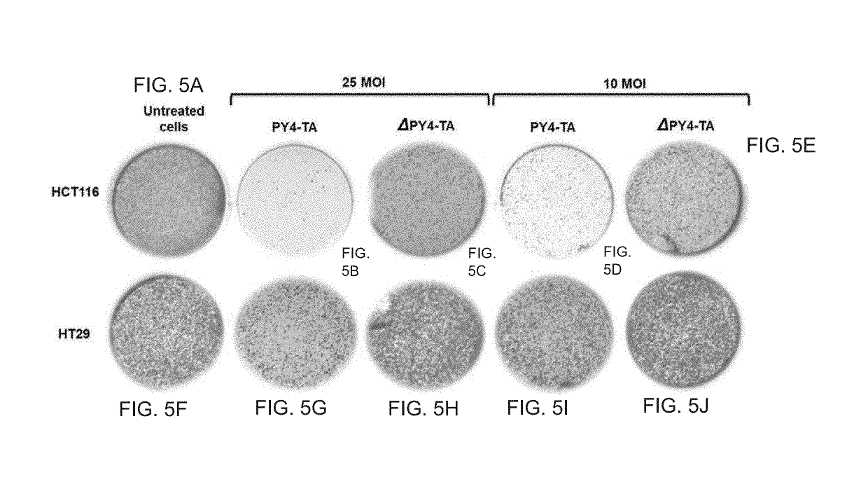

An in vitro colony-forming assay was performed to qualitatively and comparatively assess the sensitivity of CRC cells with mutated RAS to the expression of the transgene. In addition, this assay was intended to verify that MazF is well tolerated by the normal cells that do not harbor hyperactive RAS. HCT116 (harboring hyperactive RAS) and HT29 (no KRAS mutation) cells were infected with 25 and 10 MOI of “pAdEasy-Py4-TA”, “pAdEasy-ΔPy4-TA” or left uninfected. The cells were trypsinized and seeded in 3-fold dilutions 7 hours later. Surviving colonies were stained after 7 days. FIGS. 5A-J show the potency of the expressed transgene under the control of the RRE (FIG. 5B) compared to uninfected cells (FIG. 5A) and to the control cassette carrying a deletion of the RRE (FIG. 5D). Prominent differences in the numbers of surviving colonies were observed, confirming that MazF indeed overcame the inhibitory effect of MazE in mutated RAS-harboring cells (FIG. 5B) while MazE was able to protect cells (with the wild type RAS, devoid of the RA hyperactive mutation) from MazF toxicity (FIG. 5G). In addition, the selectivity of this targeting system was confirmed since the massive cell death took place only in the RRE- including cassette (FIG. 5B), while no significant effect was seen after infection with the ΔPy4-TA cassette that lacks the RRE (FIG. 5D).

Deletion of the RAS-Responsive Element Decreases the Cytotoxic Activity of mazF—

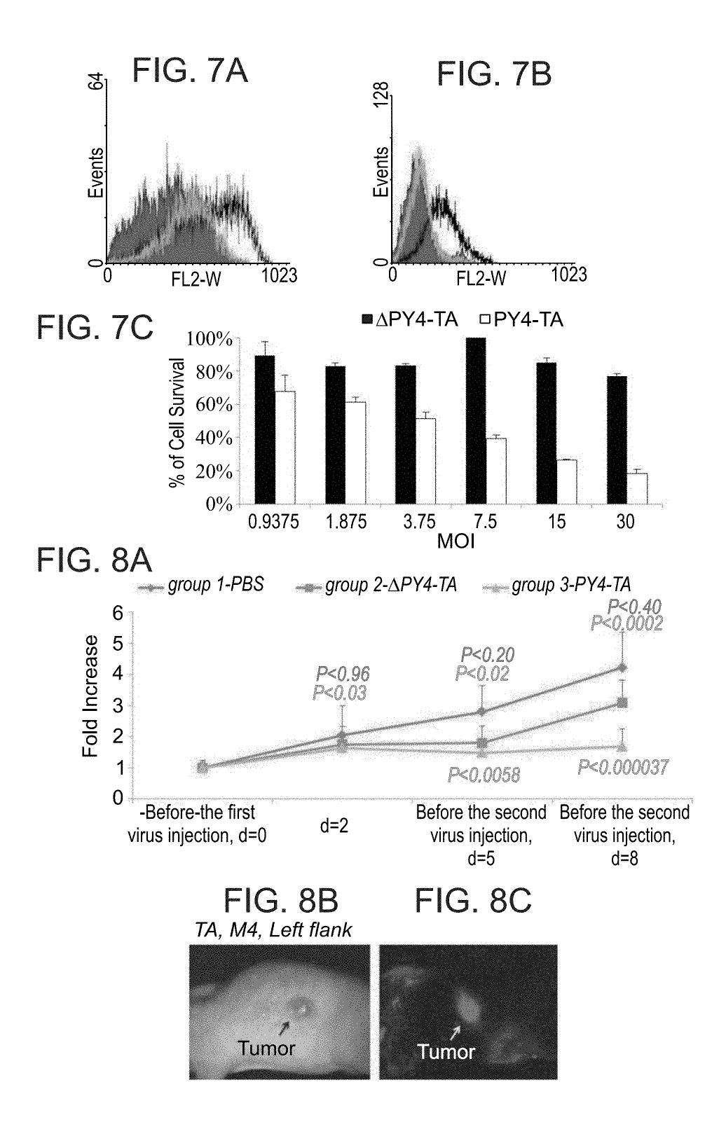

As mentioned above, an additional cassette lacking the RRE, “pAdEasy-ΔPy4-TA”, was also constructed (FIG. 2B). FIGS. 7A-C demonstrate that deletion of the RAS-responsive DNA element enhanced the contribution of MazE. Cell viability was measured by FACS analysis: while massive cell death (55% apoptosis, 82% dead cells) was observed following infection with the full toxin-antitoxin encoding viruses, deletion of the RAS-responsive DNA element dramatically reduced this effect to 18% and 10%, respectively (FIGS. 7A-B; the untreated cells are shown in red, the toxin antitoxin-treated cells are shown in black, and the cells that were treated with the RRE deletion cassette are shown in green). These results were also confirmed by the enzymatic MTT assay (FIG. 7C), where a difference of about 60% was observed between Py4-TA and ΔPy4-TA.

Adenovirus-Mediated Delivery of TA Encoding Cassette Specifically Eliminates Activated RAS-Harboring CRC Cells—

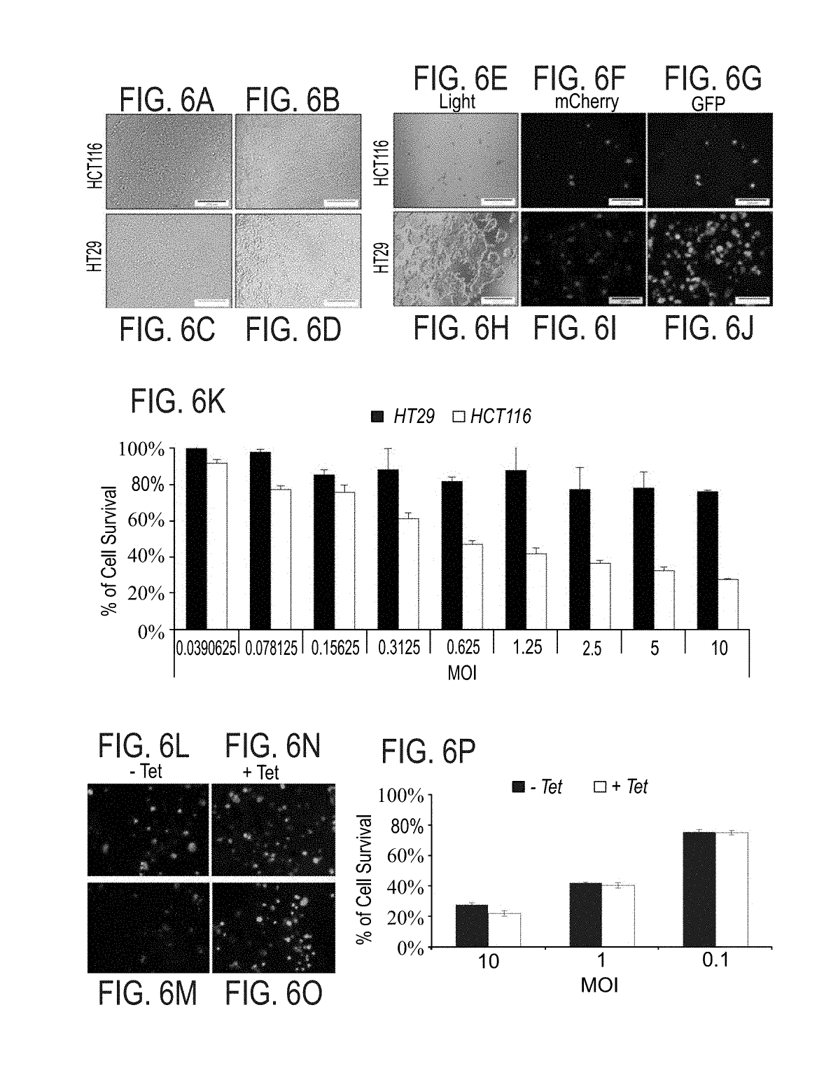

The present inventors further examined the ability of the above-described TA system to kill a human cancer cell line expressing hyperactive RAS. HCT116 and HT29 cells were infected with twofold dilutions of the MazEF-encoding viruses, starting from 10 MOI (FIGS. 6E-J), or left untreated (FIGS. 6A-D). Massive cell death (73%, relative to the uninfected control, at 10 MOI) was demonstrated exclusively in the mutated KRAS harboring cells (HCT116) but not in cells lacking hyperactive RAS (HT29 cells), emphasizing the potency of this system (FIG. 6K). The percentage and intensity of the fluorescence of green cells (FIG. 6J) was higher than that of the red ones (FIG. 6I) in HT29 cells, lacking hyperactive RAS. This indicates that the expression of the antitoxin increases and exceeds that of the toxin in cells devoid of the hyperactive RAS.

In order to confirm and further support the ability of this system to protect normal cells on one hand, and to efficiently kill cancer cells on the other hand, additional CRC cell lines were tested and yielded very similar results, substantiating the above observations (data not shown). The potency of this system was also tested for other cancer types, such as pancreatic and prostate, suggesting a wide range of therapeutic potential of this suggested treatment modality (data not shown).

Induction by Tetracycline Results Increased Expression of the Antitoxin MazE in Naïve Cells—

Inclusion of tetracycline provided an additional protective layer, not only for virus production and propagation but also for normal cell protection. Binding of tetracycline to the Tet repressor led to a conformation change that resulted in relief of the MazE transcriptional inhibition. Consequently, expression of the antitoxin was increased in naïve cells, as demonstrated by the increase in green fluorescence (FIGS. 6N and 6O), while tetracycline did not compromise the toxicity of MazF in mutated RAS-harboring cells (FIG. 6P). It is noted that in cells with mutated RAS with or without the addition of tetracycline, the viability of the cells was the same.

### Example 5

### MazEF-Encoding Viruses Inhibit Tumor Growth In Vivo

Experimental Results

MazEF-Encoding Viruses Inhibit Tumor Growth In Vivo—

The therapeutic potential of the TA system was tested by specific targeting of tumor cells in nude mice bearing a xenograft of HCT116 CRC cells, harboring mutated RAS. The growth of these cells was markedly inhibited by Py4-TA-encoding viruses (FIG. 8A). Impressive tumor shrinkage was demonstrated in vivo following treatment with Ad-Py4-SV40-MazEF-encoding adenovirus (61%) (P<0.0002) without any toxic or side effects. In the Ad-ΔPy4-SV40-MazEF treated mice (control group) tumor volume was reduced by only 27% (P<0.4). No growth inhibition was seen following injection of PBS (FIG. 8A).

Throughout this study the mice were monitored for their general well-being, weight, food and water consumption. At no time there was any evidence of toxicity ascribable to the adenoviral vectors (data not shown).

The expression of MazF and MazE in the infected tumor cells was monitored with the Maestro imaging CRi device (FIG. 8B). The imaging was performed on live mice (FIG. 8B) and outside the mouse body (FIG. 8C). This analysis confirmed that the adenoviruses indeed targeted the mutated RAS-harboring tumors and that the MazEF genes were expressed.

### Example 6

### An Orthotopic Model for Testing the Constructs

The present inventors have set up an orthotopic model for testing the efficacy of the therapeutic constructs system. For that purpose, the R1 cells (enterocytes that constitutively express mutated KRAS) are initially used. Additionally or alternatively, a similar system in murine cells (mc38 cell line) which express the KRAS mutated oncogene is currently designed.

Study Design:

The cells are injected into the colon of the mice; the initial volume of the tumors is measured by colonoscopy and then the treatment by a systemic injection of the viruses begins.

Since the orthotopic model is performed in C57/bl mice, the present inventors first tested whether injection of the R1 cell line can grow to form tumors in the mouse. For that purpose, 5×10⁶ R1 cells were subcutaneously injected at two sites on the backs of the mice. All the mice (5/5) developed tumors (data not shown). The viruses used for this experiment were propagated, produced and their titer was determined.

### Example 7

### Establishment of Stable Transfected System for Kras Mutation

The present inventors have designed and constructed an additional vector which encodes for the mutated has having a missense mutation G13D in the KRAS (FIG. 9A). FIG. 9B schematically illustrates the luciferase construct. Next, a stable transfection to the mc38 murine colon carcinoma cells was performed and by measuring the luciferase expression the best clone was screened and selected (FIG. 9B), and as shown, the best clone was C3.

### Example 8

### AAV (Adeno Associated Virus)

The limitations of using adenovirus are well known, among other reasons because it causes an immune response. Therefore, the present inventors currently establish less immunogenic delivery systems; such as the AAV delivery system.

The AAV is one of the smallest ‘viruses, it is a single-stranded DNA. Its properties made it one of the most promising delivery systems, partly because of its low immunogenicity, the long duration of transgenic expression, and because there are many serotypes to AAV and each one of them can infect only specific cell type, therefore the selectivity of the virus is high.

Identification of the Most Appropriate Serotype for the Different Target Cells, Especially Colorectal and Pancreatic Cancer Cells—

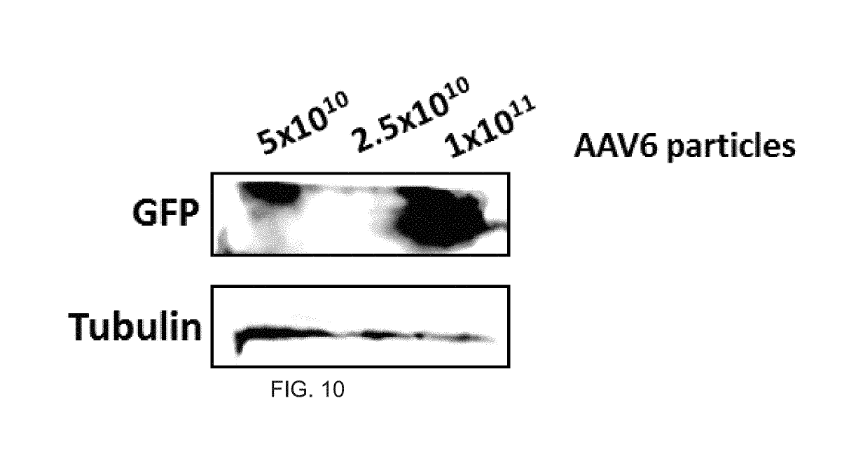

For that purpose, the present inventors induced tumors that derived from different cell lines in nude mice. Then a systemic single infecting of the various serotypes in several titers were conducted, and after two weeks the mice were sacrificed, the tumors were removed and the expression of the GFP was evaluated by Western blot analysis as shown in FIG. 10. According to these results, the AAV serotype 6 is the best serotype for the HT29 target cells.

The PY4-mazEF and ΔPY4-mazEF cassettes were cloned to AAV serotype 6 and the viruses are produced. These particles are evaluated in vivo, in nude mice bearing xenograft of pancreatic cancer cells.

### Example 9

### Establishment of a Dual System Based on RAS and P53 Responsive Elements

For establishing the dual system adenoviral vectors carrying the toxin (PY4-MazF-mcherry) and the antitoxin (RGC-MazE-IRES-GFP) were cloned under the regulation of Ras responsive elements and p53 responsive elements, respectively. FIG. 11A provides a schematic illustration of such a dual system.

Virus particles were produced, their titer was calculated and their potency was tested in vitro. Cell death was measured qualitatively by using the fluorescent microscopy and was quantified by the enzymatic MTT assay.

A594, Ras^{mut}/p53^{wt}; H2030, Ras^{mut}/p53^{wt}; H1299, Ras^{mut}/p53^{wt}; H1650, Ras^{wt}/p53^{wt} and H1975, Ras^{wt}/p53^{mut}—lung cancer (LC) cell lines were used as a model system for testing the potency of the adenoviruses-based system. Mia Paca, Ras^{mut}/p53^{mut}; Colo357, Ras^{mut}/p53^{mut} Panc1, Ras^{mut}/p53^{mut} and BxPC3, Ras^{wt}/p53^{mut}—pancreatic cancer cell lines were tested as well.

Co-infection assays were performed, using the optimal 1:0.5 MOI ratio. The results showed decrease in the mortality of the mutated Ras cells expressing wild type (WT) p53; 36% with a titer of 7.5 MOI (FIG. 12). These results indicate that cells, which have WT p53, that expressed the toxin were protected by the antitoxin expressed under the p53 responsive element, while cells that have mutations of both genes, i.e., ras and p53, such as the SHP77, showed increased sensitivity.

The efficacy of the above dual system is tested in vitro and in vivo.

### Example 10

The present inventors further tested whether the RAS responsive PY4 element works as an enhancer only in cells that carry mutation of the KRAS or even of N- and H-RAS genes. For that purpose, the ability of all the three RAS oncogenes (HRAS, NRAS, and KRAS) to activate their pathways was examined by testing their ability to stimulate their downstream transcription factors. H1299 (NRAS oncogene expressing cell line), A549 (KRAS oncogene expressing cell line) and T24 (HRAS oncogene expressing cell line) were co-transfected with PY4-luciferase and Renila luciferase plasmids. The results presented in FIG. 13A showed that the all three are able to induce the transcription of the luciferase reporter gene, with different efficiency (transcription levels of PY4-luciferase were normalized to the Renila luciferase activity).

Next, the present inventors examined the ability of those three oncogenes to induce the transcription of the toxin under the regulation of PY4 element. H1299, H2030, A549, and T24 were infected with Ad-PY4-mcherry-mazF viruses. Cell viability was measured by the MTT assay (72 hours post infection). These experiments support the previous observations by showing the different levels of toxin expression which in turn are leading to different percentage of cell viability (FIG. 13B).

### Example 11

### Chemical Induction of P53 Expression in CRC Cells P53 Responsive Element

Experimental Results

In order to test the ability of p53 to bind to its responsive element and stimulate transcription, HCT116 cells were transfected with RGC-mazE-IRES-GFP plasmid. Since p53 is degraded in un-activated cells, the present inventors used Quercetin, a ubiquitous bioactive plant flavonoid that is able to induce p53 phosphorylation, stabilization and total p53 protein accumulation. Another widely used p53 inducer is the antimetabolite agent 5-FU. P53 transcription activity was evaluated by measuring the expression of the downstream reporter gene, GFP. The present inventors show that the addition of Quercetin (50 μM) to the transfected HCT116 cells increased the expression of GFP by about 10 folds. Addition of 5-FU has led to significantly higher expression levels of GFP, even in lower concentration (10 μM). Furthermore, Quercetin showed severe cell toxicity (60% cell death) while no toxicity has been shown upon treatment with 5-FU, even very high doses (data not shown).

Induction of the Transcription of P53 Target Genes—

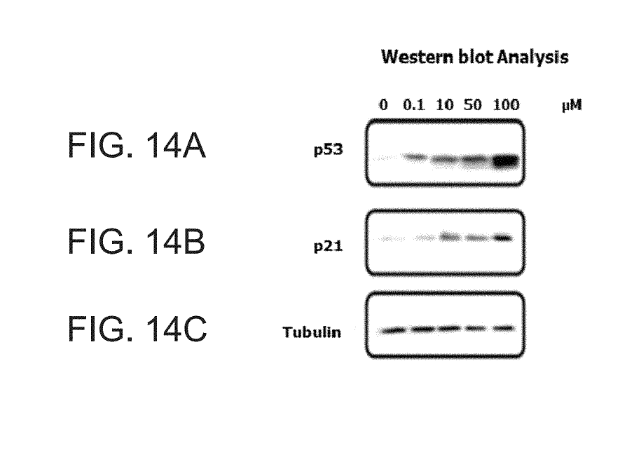

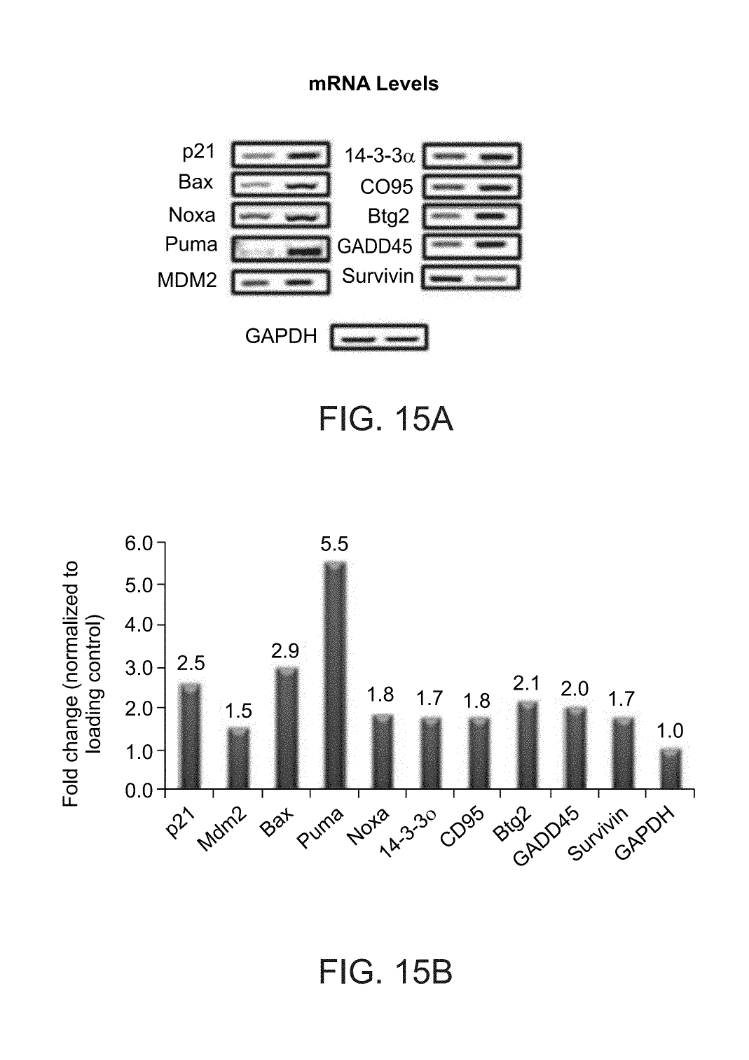

Further validation of endogenous p53 activation led the present inventors to test other canonical target genes. 5-FU (50 μM) was added to HCT116 p53 WT cells for 24 hours. Total cell lysate was prepared and subjected into Western blot analysis and at the same time RNA was prepared and used as a template for cDNA and semi-quantitative PCR was performed. The results show and confirm that protein expression, of all the target genes, correlates to the mRNA up regulation due to p53 activation. In particular a tight correlation between p21 and p53 protein levels (FIGS. 14A-C).

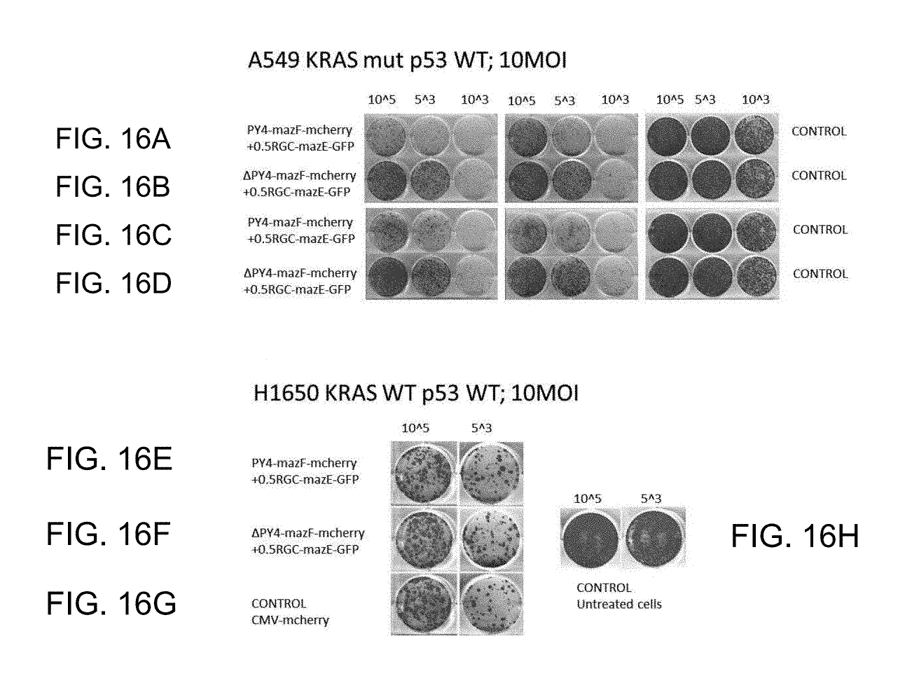

An in vitro colony-forming assay was performed to qualitatively and comparatively assess the sensitivity of lung cancer cells with mutated RAS to the expression of the transgene. In addition, this assay was intended to verify that MazF is well tolerated by the normal cells that do not harbor hyperactive RAS. A549 and H1650 cells were infected with 10 MOI of the adeno-PY4-mazF-mcherry and adeno-RGC-mazE-GFP viruses, in a ratio of 1:0.5 ratio, respectively. In parallel, those cells were infected with adeno-ΔPY4-mazF-mcherry and adeno-RGC-mazE-GFP viruses, in a ratio of 1:0.5, respectively. Empty vector (CMV-mcherry) infected or uninfected cells used as a control. After 7 hours, the cells were trypsinized and seeded in 3-fold dilutions and incubated for 7 days. Surviving colonies were fixed with 4% formaldehyde in PBS and stained with 0.02% crystal violet. A549 KRAS mutated and p53 WT expressing cells that were infected with PY4-mazF-mcherry and RGC-mazE-GFP viruses showed lower survival rate compared to ΔPY4-mazF-mcherry and RGC-mazE-GFP viruses infected cells. However, H1650 KRAS WT and p53 WT expressing cells that were infected with PY4-mazF-mcherry and RGC-mazE-GFP viruses showed no significant fold change in survival compared to ΔPY4-mazF-mcherry and RGC-mazE-GFP viruses infected cells (FIGS. 16A-H).

A549 cells express the mutated KRAS oncogene therefore the expression of the mazF toxin is high. In addition, these cells carry WT form of p53 that binds to its responsive element and enhances the transcription of the mazE anti-toxin. On the contrarily, H1650 cells express WT RAS, therefore the transcription of the toxin wasn't enhanced. Consequently, there was not a significant difference in toxicity between cells that were infected with viruses that carry or not the RAS responsive elements. The presence of WT p53 in these cells leads to the expression of the anti-toxin. Both the toxin and the antitoxin were visualized by the expression of the fluorescence proteins (mcherry and GFP, respectively). It is important to note the two cell lines above are different in their sensitivity to viral infections. H1650 cells showed much lower survival level then A549 line, upon infection with an empty vector (FIGS. 16A-H).

The Efficacy of mazF to Kill has been Evaluated Also in Pancreatic Cancer Cells—

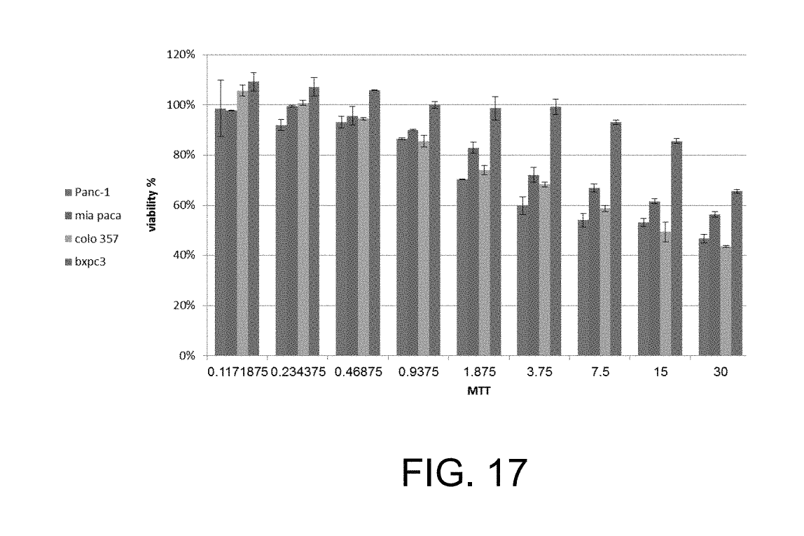

PANC1, Mia Paca2, Colo357 (KRAS mutated cells) and BxPC3 (wt RAS) cell lines were seeded in 96-well plates. After 24 hours different dilutions of PY4-mazF-mcherry viruses were added. 72 hours later, cell survival was measured the enzymatic MTT assay. The results show that cells with hyperactive KRAS were more sensitive, about 50% viability in MOI of 15. However, WT RAS expressing cells showed 80% viability under the same conditions (FIG. 17).

Analysis and Discussion

In the present study vectors for cancer-directed gene delivery were constructed; “pAdEasy-Py4-SV40mP-mCherry-MazF”, “pAdEasy-Py4-SV40mP-mCherry-MazF-IRES-TetR-CMVmp-MazE-IRES-EGFP”, “pAdEasy-ΔPy4-SV40mP-mCherry-MazF-IRES-TetR-CMVmp-MazE-IRES-EGFP” and “pAdEasy-mCherry”. Virus particles were produced and their potency was tested. Cell death was measured qualitatively by using the fluorescent microscopy and colony formation assay, and was quantified by MTT. FACS analysis using annexin V and RedDot2 dyes was performed for measuring apoptosis and dead cells, respectively. In vivo tumor formation was measured in xenograft model.

Herein, an improved approach is suggested by tightening the expression of the toxin and replacing the pro-apoptotic gene by a significant more potent toxic molecule that does not exist in human cells.

These results demonstrate a very well-regulated system that can precisely control gene delivery and expression at a specific targeted site. This system exploits the hyperactive RAS pathway, rather than inhibiting it. In addition, the results presented here demonstrate a proof-of-concept that normal tissues can be selectively spared from the toxic effects of drugs by taking advantage of their wt RAS that express the “antidote”.

Thus, the MazF-MazE toxin-antitoxin system has a potential to be used as a therapeutic tool, to kill undetectable micrometastases that are a major hurdle in challenging.

This approach, of taking advantage of Ras mutation in order to selectively kill cancer cells while sparing the normal cells, either alone or preferably in conjunction with other treatment modalities can enhance efficacy while reducing toxicity.

Thus, the suggested gene therapy strategy can advance the management of human cancer, allowing a tailor-made protocol for biological treatment specific to the molecular profile of a tumor.

### Example 12

### Delivery of the Therapeutic Constructs According to Some Embodiments of the Invention Using Exosomes

Colony Formation Assay

Target cancer cells harboring KRAS mutation at 5×10⁵ per well are seeded in 6 wells plate. 24 h later, cells are treated with different amounts of exosomes loaded with the different cassettes, such as described above, or left untreated. Following 24 hours, cells are trypsinized and seeded in 3 fold dilutions and incubated for 7-10 days. Surviving colonies are fixed with 4% formaldehyde in PBS and stained with 0.02% crystal violet.

Functional Assays

The cell killing activity of MazF- and MazE-encoding exosomes is measured by the MTT assay.

MazF-induced cytotoxicity is also determined by ³H-Leucine incorporation. In addition, apoptosis is detected using the Annexin V PI staining (Annexin V Apoptosis kit, MBL) and cell cycle arrest by using propidium iodide (PI) staining for analysis of DNA content. The number of subdiploid cells, representing apoptotic cells, is quantified by FACS.

Delivery of the Toxin-Antitoxin Exosomes into Cancerous Cells of Interest

To increase the specific targeting of the exosomes expressing the toxin-anti-toxin constructs into the cancerous cells of interest, e.g., cancerous cells which express CD24 on their membrane, the present inventors have devised cell lines which constitutively express anti-CD24 antibody on the cell membrane, such that exosomes generated by such cells also present the CD24 antibody on their membrane. The exosomes presenting the CD24 antibody can be then purified and further transfected [e.g., using the Exo-Fect™ transfection kit (System Biosciences)] with the constructs of some embodiments of the invention which include the toxin-antitoxin sequences. Using this system the exosomes harboring the toxin-antitoxin system are directly delivered to cancerous cells expressing CD24.

Construction of scFv or Fab that are Expressed on the Exosome Membrane

Nucleic acid sequences of the scFV, FAB or the anchoring sequence (e.g., transmembrane domain or a viral envelop glycoprotein) are codon optimized for better expression in mammalian systems. The gene, which is encoding the scFv or Fab of the anti-CD24 mAb, is synthesized and cloned into the respective expression vector. The construct which produces the small fragment antibody further includes a membrane anchoring sequence such as a transmembrane domain of a membrane protein such as that of EGFR, PDGFR or FGFR (that anchors the antibody to allow the flexibility of the antibody fragment to reach its target, and thus reduce steric hindrance); or alternatively an anchoring sequence from a viral envelope protein such as of VSV-G or GPI anchoring proteins. The construct is used to generate a stable cell line (e.g., HEK293 or T-REx-293 cell line) that constitutively expresses the above antibody and produces the modified exosomes (wherein the scFv or Fab of the anti-CD24 mAb is presented on the exosome membrane).

I. A first type of exosome is obtained by transfecting the cells with a construct that includes the following nucleic acid sequences (from the 5′ to the 3′) encoding the following domains: signal peptide, HA tag, restriction enzyme site, scFv(CD24) sequence, restriction enzyme site, HIS×6 tag, G₄S (glycine-serine) linker, transmembrane domain.

The scFv includes the V_{H} and V_{L} sequences and linker of G₂SG₄SG₃ between them.

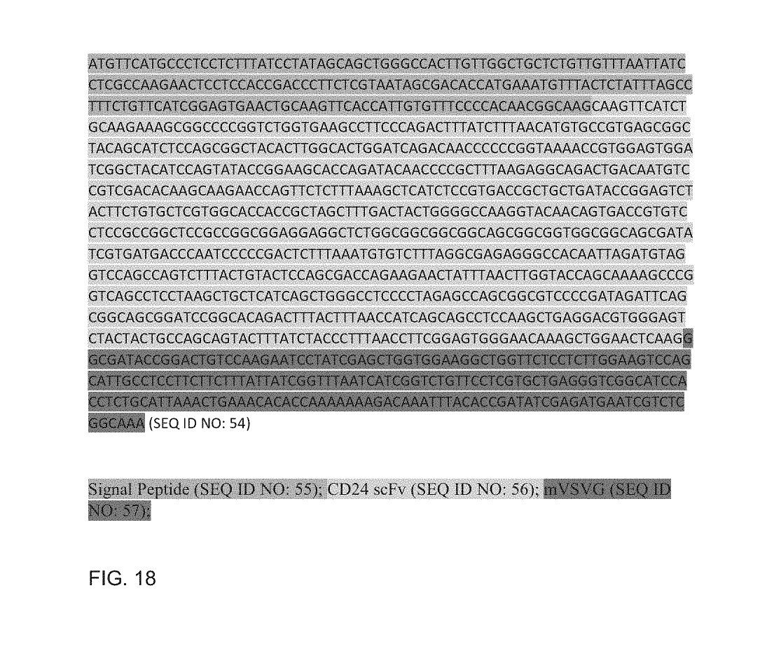

II. A second type of exosome is obtained by transfecting the cells with a construct which includes the scFv(CD24) coding sequence (e.g., SEQ ID NO: 56) further includes the coding sequence of at least a partial sequence of the viral envelop glycoprotein such as the VSV-G envelop glycoprotein (SEQ ID NO: 57). Such a partial sequence of the VSV-G envelop glycoprotein is capable of anchoring the scFv (CD24) antibody within the cell membrane, so as to increase presentation of the CD24 antibody on the cell membrane. The construct can further include a signal peptide upstream of the CD24 scFv (SEQ ID NO: 55). A representative polynucleotide sequence which includes the signal peptide, the CD24 scFV and the minimal sequence VSV-G is depicted in FIG. 18 and is set forth by SEQ ID NO: 54.

Screening for Clones Having Superior Targeting Ability—

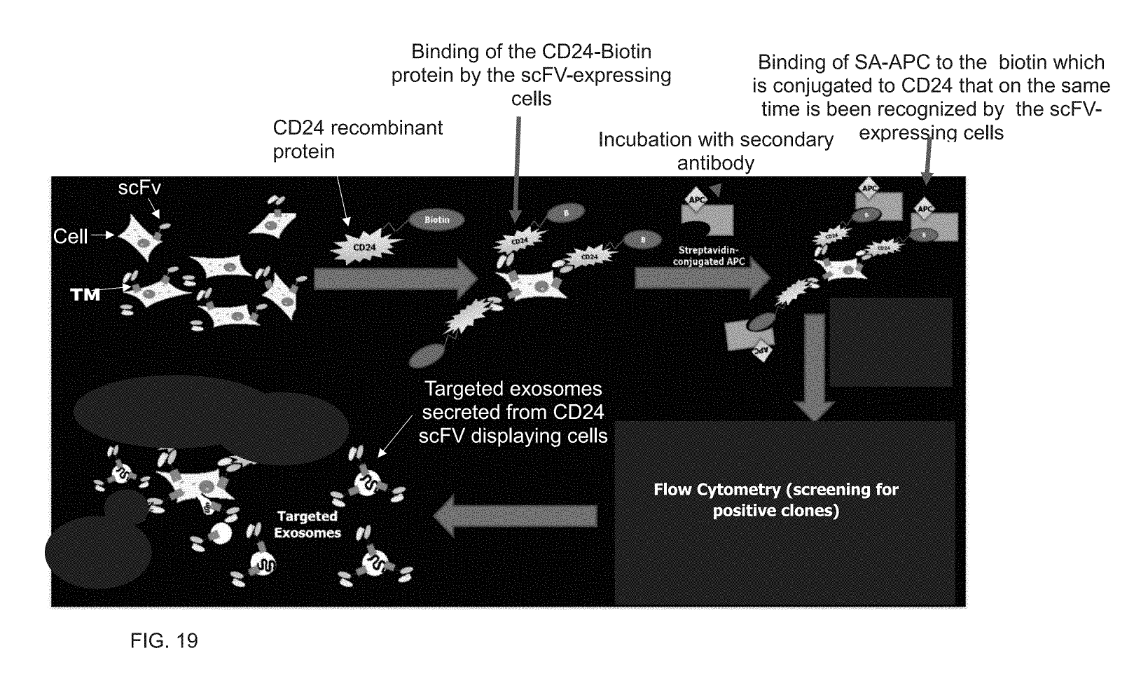

In order to screen for the best clone, a bioassay was developed. A schematic illustration of the method is depicted in FIG. 19.

Expressing vectors (pCDNA4/TO) were generated with a polynucleotide sequence comprising the CD24 scFV and the mVSV-G (e.g., the polynucleotide set forth by SEQ ID NO: 54) and were used to transform T-REx-293 cells by stable transformation. The stable clones display the CD24 scFv. The cells are incubated with the recombinant CD24 protein that is fused to a biotin molecule. Then, a Streptavidin-conjugated Allophycocyanin (APC) antibody is added (which binds to the biotinylated CD24 that is bound the cells via the scFv) and the detection of cells expressing CD24 scFV antibody is done by Flow cytometry.

FIG. 19 schematically illustrates the main steps of generating exosomes which are targeted to cancer cells according to some embodiments of the invention.

Once stable clones are elected, exosomes can be purified from these stable clone cells. Such exosomes express the scFv CD24 antibody on their cell membrane. The exosomes are purified and isolated. Once isolated, the CD24 expressing exosomes are loaded with the nucleic acid constructs comprising the toxin and anti-toxin sequences [which can be labeled with the mCherry (for the toxin construct) and the GFP (for the anti-toxin construct)] such the cell-free particle (e.g., exosome in this case) of some embodiments of the invention includes both the toxin-anti toxin system and the targeting moiety (e.g., CD24 scFV) to cancerous cells.

Purification of Exosomes:

Polymer-Based Precipitation of Exosomes

The conditioned medium of HEK293 cell line (stably transfected with the construct of the scFv) is replaced to exo-free FBS and then the cells are cultured for additional 3-4 days and then collected and centrifuged at 3000×g for 15 minutes to remove cells and cell debris. Then the supernatant is transferred to a sterile vessel and an appropriate volume of precipitation solution (according to the manufacturer's instructions, when using the ExoQuick-TC (SBI), 1 ml of precipitation solution is added for each 5 ml of sample volume is added). After at least 12 h of incubation at 4° C., the mixture is centrifuged and the exosomes appear as a beige or white pellet at the bottom of the tube after which they are resuspended is PBS. Additionally, the exosomes are purified by passing the precipitate on a column (magnetic beads or Ni⁺² beads) fused with the antibody's epitope (HIS×6 tag) so as to remove non-specific binding or exosomes that do not have antibody fragments on their surface, followed by their elution from the column.

Differential Centrifugation

Differential centrifugation remains one of the most common techniques of exosome isolation. The method consists of several steps, including 1) a low speed centrifugation to remove cells and apoptotic debris, 2) a higher speed spin to eliminate larger vesicles and finally, 3) high speed centrifugation to precipitate exosomes. According to the most common protocol, exosome isolation is realized through four consequent centrifugation steps: 10 min at 300 g, 10 min at 2000 g. The low speed centrifugation is aimed at pelleting the cells, cell debris and large vesicles. Then 30 min at 10000 g, is intended to pellet the vesicles with diameters exceeding 100-150 nm, presumably corresponding to the population of shedding vesicles. Then exosome pelleting by centrifugation at 100000 g for 70 min. A repeated 100000 g centrifugation of the re-suspended pellet is usually applied for to purify the exosome preparation from the lower mobility fractions, mainly from free proteins.

Importantly, the viscosity of the conditioned medium has a significant correlation with the purity of isolated exosomes.

Density Gradient Centrifugation

This approach combines ultracentrifugation with sucrose density gradient (Witwer K, et al., J Extracell Vesicles. 2013; 2). More specifically, density gradient centrifugation is used to separate exosomes from non-vesicular particles, such as proteins and protein/RNA aggregates. Thus, this method separates vesicles from particles of different densities. The adequate centrifugation time is very important, otherwise contaminating particles may be still found in exosomal fractions if they possess similar densities.

The gradient is generated by diluting a stock solution of 60% (w/v)) with 0.25 M sucrose, 10 mM Tris-HCl, pH 7.5 (40% (w/v), 20% (w/v), 10% (w/v) and 5% (w/v) solutions of iodixanol. These solutions are made immediately prior to centrifugation, and allow up to 30 min mixing time for each solution. The gradient was formed by adding 3 ml of 40% iodixanol solution, followed by careful layering of 3 ml each of 20% and 10% solutions, and 2 ml of the 5% solution. Concentrated culture medium was overlaid onto the top of the gradient, and centrifugation at 100,000×g (swinging bucket) for 18 h at 4° C. is performed. After centrifugation, 12 individual 1 ml gradient fractions are collected manually (with increasing density—top-bottom collection). Fractions are diluted with 2 ml PBS and centrifuged at 100,000×g for 3 h at 4° C. followed by washing with 1 ml PBS, and re-suspension in 50 μl PBS.

Exosome Quantification

The ExoELISA kit (SBI) is used to quantify the purified exosomes. Briefly, it is designed as a direct Enzyme-Linked ImmunoSorbent Assay (ELISA). The exosome particles and their proteins are directly immobilized onto the wells of the microtiter plate. After binding, wells are coated with a block agent to prevent non-specific binding of the detection antibody. The detection (primary) antibody is added to the wells for binding to specific antigen (e.g. CD63, a known exosomal marker) protein on the exosomes. A Horseradish Peroxidase enzyme (HRP) linked secondary antibody (goat anti-rabbit) is used for signal amplification and to increase assay sensitivity. A colorimetric substrate (TMB) will be used for the assay read-out. The accumulation of the colored product is proportional to the specific antigen present in each well. The results are quantitated by a microtiter plate reader at 450 nm absorbance and calibrated by the exosome standards.

Toxicity Test in Mice

Exosomes toxicity is evaluated in BALB/c mice (6-8 weeks old), that will be given a single i.p. injection with different amounts of each exosome/200 μl PBS. Mice are monitored for survival, weight loss, blood counts, and signs of distress or death for 2 weeks after injection. All tissues are examined for pathology by the end of the experiment.

Biodistribution Study

To confirm that the exosomes reach the tumor and accumulate there, exponentially growing target cells are injected subcutaneously at one site on the back of nude mice. The exosomes, loaded with the engineered DNA cassette, are administrated via one intraperitoneal injection. The distribution of the exosomes is monitored by live imaging using the IVIS device that quantifies the intensity of the fluorescent signal. Another option for monitoring the exosomes is using RT-PCR that ensures the expression of the fluorescence reporter genes.

Combined Experiments, Exosomes and Chemotherapy

A calibration study is performed to find the lowest effective exosome concentration. Tumor development, tissue distribution of the exosomes, histological examination of the tumor and the expression of mazF and mazE are measured. In an additional set of experiments, the present inventors compare mutant p53 tumor cells with wt p53 controls. CRC are tested alone by treating with SV40-mazF-loaded exosomes only, RGC-MazE-loaded exosomes only, or a mixture of exosomes together in the presence of 5-FU. Cell killing is monitored as above, and various features of p53 activation. For the combined experiments, in vivo, each group receives a suspension of exosomes, in a does as defined in the calibration test and depending on the model of malignancy. Chemotherapeutic agents are given i.p. and in dependence of the type of cancer.

Basic Protocol for Chick Chorioallantoic Membrane Assay (CAM Assay)

Fertilized eggs are incubated until day 3 (37° C., 75-90% humidity). 2 ml of yolk are pulled from the egg using a syringe, in order to separate the CAM membrane from the egg shell. Then, a small window in the egg shell is made, and resealed with adhesive tape, in order to reduce dehydration and contamination, and eggs will be returned to the incubator until day 7 of chick embryo development.

On day 7, cancer cells suspension (3-5×10⁶) are mixed with matrigel (1:1) in a total volume of 30μl. Matrigel grafts are placed on top of the CAM and eggs are resealed and returned to the incubator for 3 days when a large vascularization appears around the cells, and 3D cancerous tumor appears.

On day 10 the tested exosomes are applied and then evaluation of the responsiveness of the tumor to treatment, as compared to control un-treated tissue, is performed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. American Cancer Society. Colorectal Cancer Facts & Figures. 2014.
2. Reddy M A, Langer S J, Colman M S, et al. An enhancer element responsive to ras and fms signaling pathways is composed of two distinct nuclear factor binding sites. Mol Endocrinol 1992; 6:1051-60.
3. Nabel G J. Genetic, cellular and immune approaches to disease therapy: past and future. Nat Med 2004; 10:135-41.
4. Kootstra N A, Verma I M. Gene therapy with viral vectors. Annu Rev Pharmacol Toxicol 2003; 43:413-39.
5. Verma I M, Weitzman M D. Gene therapy: twenty-first century medicine. Annu Rev Biochem 2005; 74:711-38.
6. Young L S, Searle P F, Onion D, et al. Viral gene therapy strategies: from basic science to clinical application. J Pathol 2006; 208:299-318.
7. Kaplan J M. Adenovirus-based cancer gene therapy. Curr Gene Ther 2005; 5:595-605.
8. El-Aneed A. Current strategies in cancer gene therapy. Eur J Pharmacol 2004; 498:1-8.
9. Dvory-Sobol H, Kazanov D, Arber N. Gene targeting approach to selectively kill colon cancer cells, with hyperactive K-Ras pathway. Biomed Pharmacother 2005; 59 Suppl 2:S370-4.
10. Dvory-Sobol H, Sagiv E, Kazanov D, et al. Targeting the active beta-catenin pathway to treat cancer cells. Mol Cancer Ther 2006; 5:2861-71.
11. Dvory-Sobol H, Sagiv E, Liberman E, et al. Suppression of gastric cancer cell growth by targeting the beta-catenin/T-cell factor pathway. Cancer 2007; 109:188-97.
12. Giladi N, Dvory-Sobol H, Sagiv E, et al. Gene therapy approach in prostate cancer cells using an active Wnt signal. Biomed Pharmacother 2007; 61:527-30.
13. Naumov I, Kazanov D, Lisiansky V, et al. Novel approach to abuse the hyperactive K-Ras pathway for adenoviral gene therapy of colorectal cancer. Exp Cell Res 2012; 318:160-8.
14. FitzGerald D, Pastan I. Redirecting Pseudomonas exotoxin. Semin Cell Biol 1991; 2:31-7.
15. Lisiansky V, Naumov I, Shapira S, et al. Gene therapy of pancreatic cancer targeting the K-Ras oncogene. Cancer Gene Ther 2012; 19:862-9.
16. Inouye M. The discovery of mRNA interferases: implication in bacterial physiology and application to biotechnology. J Cell Physiol 2006; 209:670-6.
17. Pandey D P, Gerdes K. Toxin-antitoxin loci are highly abundant in free-living but lost from host-associated prokaryotes. Nucleic Acids Res 2005; 33:966-76.
18. Engelberg-Kulka H, Amitai S, Kolodkin-Gal I, et al. Bacterial programmed cell death and multicellular behavior in bacteria. PLoS Genet 2006; 2:e135.
19. Engelberg-Kulka H, Hazan R, Amitai S. mazEF: a chromosomal toxin-antitoxin module that triggers programmed cell death in bacteria. J Cell Sci 2005; 118:4327-32.
20. Cook G M, Robson J R, Frampton R A, et al. Ribonucleases in bacterial toxin-antitoxin systems. Biochim Biophys Acta 2013; 1829:523-31.
21. Thisted T, Sorensen N S, Wagner E G, et al. Mechanism of post-segregational killing: Sok antisense RNA interacts with Hok mRNA via its 5′-end single-stranded leader and competes with the 3′-end of Hok mRNA for binding to the mok translational initiation region. EMBO J 1994; 13:1960-8.
22. Gerdes K, Bech F W, Jorgensen S T, et al. Mechanism of postsegregational killing by the hok gene product of the parB system of plasmid R1 and its homology with the relF gene product of the E. coli relB operon. EMBO J 1986; 5:2023-9.
23. Zhang Y, Zhang J, Hoeflich K P, et al. MazF cleaves cellular mRNAs specifically at ACA to block protein synthesis in Escherichia coli. Mol Cell 2003; 12:913-23.
24. Shapira A, Shapira S, Gal-Tanamy M, et al. Removal of hepatitis C virus-infected cells by a zymogenized bacterial toxin. PLoS One 2012; 7:e32320.
25. Arber N, Sutter T, Miyake M, et al. Increased expression of cyclin D1 and the Rb tumor suppressor gene in c-K-ras transformed rat enterocytes. Oncogene 1996; 12:1903-8.
26. Okamoto M, Chono H, Kawano Y, et al. Sustained inhibition of HIV-1 replication by conditional expression of the E. coli-derived endoribonuclease MazF in CD4+ T cells. Hum Gene Ther Methods 2013; 24:94-103.
27. Chono H, Matsumoto K, Tsuda H, et al. Acquisition of HIV-1 resistance in T lymphocytes using an ACA-specific E. coli mRNA interferase. Hum Gene Ther 2011; 22:35-43.
28. Shimazu T, Mirochnitchenko O, Phadtare S, et al. Regression of Solid Tumors by Induction of MazF, a Bacterial mRNA Endoribonuclease. J Mol Microbiol Biotechnol 2014; 24:228-33.

What is claimed is:

1. A cell-free particle comprising a nucleic acid sequence encoding a toxin and a nucleic acid sequence encoding an anti-toxin and wherein said particle comprises a targeting moiety for delivery of the particle into a cancer cell.

2. The cell-free particle of claim 1, comprising a nucleic acid construct system comprising:
(i) a first nucleic acid construct encoding said toxin operatively linked to a first promoter and at least one cancer-associated signaling responsive enhancer element;
(ii) a second nucleic acid construct encoding said anti-toxin operatively linked to a second promoter, said second promoter being stronger than said first promoter.

3. The cell-free particle of claim 1, comprising a nucleic acid construct system comprising:
(i) a first nucleic acid construct encoding a toxin operatively linked to a first promoter and at least one cancer-associated signaling responsive enhancer element; and
(ii) a second nucleic acid construct encoding an anti-toxin operatively linked to a second promoter;
wherein said first nucleic acid construct is provided at a higher concentration than said second nucleic acid construct.

4. The cell-free particle of claim 3, wherein said first promoter and said second promoter are identical promoters.

5. The cell-free particle of claim 1, wherein said toxin and said anti-toxin are selected from the group consisting of MazF/MazE, kid/kis, CcdB/CcdA, ChpBK/ChpBI, RelE/RelB, ParE/ParD, HipA/HipB, PhD/Doc, Hok/Sok, YafM/YoeB, YafN/YafO, YgjM/YgjN, YgiT/YgiU, DinJ/YafQ, VapB/VapC, HipB/HipA, and HicB/HicA.

6. The cell-free particle of claim 1, wherein said anti-toxin is translationally fused to a destabilization sequence.

7. The cell-free particle of claim 1, wherein said targeting moiety is selected from the group consisting of an antibody, an antibody fragment, a peptide and an aptamer.

8. The cell-free particle of claim 7, wherein said antibody or antibody fragment is encoded from a polynucleotide comprising a nucleic acid sequence encoding said antibody or antibody fragment translationally fused upstream of a nucleic acid sequence encoding a membrane-anchored amino acid sequence.

9. The cell-free particle of claim 8, wherein said membrane-anchored amino acid sequence comprises at least a partial transmembrane and an extracellular amino acid sequence of a retrovirus envelop glycoprotein.

10. The cell-free particle of claim 9, wherein said retrovirus is VSV and wherein said envelop glycoprotein is set forth by SEQ ID NO: 58.

11. The cell-free particle of claim 9, wherein said retrovirus is VSV and wherein said partial transmembrane and an extracellular amino acid sequence of said envelop glycoprotein is encoded by the nucleic acid sequence set forth by SEQ ID NO: 57.

12. The cell-free particle of claim 8, wherein said polynucleotide further comprises a nucleic acid sequence encoding a signal peptide being translationally fused upstream of said nucleic acid sequence encoding said antibody or said antibody fragment.

13. The cell-free particle of claim 7, wherein said targeting moiety specifically binds a tumor marker.

14. The cell-free particle of claim 13, wherein said tumor marker is selected from the group consisting of CD24, AFP, αᵥβ₃ (vitronectin receptor), CA125 (MUC16), CD4, CD20, CD22 (Siglec-2), CD30 (TNFRSF1), CD33 (Siglec-3), CD52 (CAMPATH-1), CD56 (NCAM), CD66e (CEA), CD80 (B7-1), CD140b (PDGFRβ), CD152 (CTLA4), CD227 (PEM, MUC1, mucin-1), EGFR (HER1, ErbB1), EpCam, GD3 ganglioside, HER2 (HER2/neu,ErbB2), PSMA, Sialyl Lewis, VEGF, E-cad, CLDN7, FGFR2b, N-cad, Cad-11, FGFR2c, EGFR, FGFR1, FOLR1, IGF-I Ra, GLP1R, PDGFRa, PDGFRb, TNFRSF11b, EPHB6, VEGFR, ABCG2, CXCR4, CXCR7, integrin-αvβ3, SPARC, VCAM, ICAM and CD44.

15. The cell-free particle of claim 1, wherein the particle has a diameter of about 30-120 nm.

16. The cell-free particle of claim 1, wherein said cell-free particle is comprised in a cell free sample in which the majority of protein is comprised in cell-free particles comprising a plurality of said cell-free particle.

17. The cell-free particle of claim 1, being an exosome.

18. The cell-free particle of claim 1, being derived from a cell selected from the group consisting of a tumor cell, a stem cell, healthy cell, stably transfected cell.

19. The cell-free particle of claim 18, wherein said cell is a human cell.

20. The cell-free particle of claim 2, wherein said second promoter comprises CMV and said first promoter comprises SV40.

21. The cell-free particle of claim 2, wherein said cancer-associated signaling responsive enhancer element comprises a Ras-responsive element.

22. The cell-free particle of claim 2, wherein said first nucleic acid construct and said nucleic acid construct are co-transfected into cells at a 1 to 0.5 ratio, respectively.

23. The cell-free particle of claim 21, wherein said Ras comprises K-Ras.

24. The cell-free particle of claim 2, wherein said toxin and said anti-toxin comprise a bacterial-derived toxin anti-toxin system.

25. The cell-free particle of claim 2, wherein said toxin anti-toxin system comprise a MazEF system.

26. The cell-free particle of claim 2, wherein said second nucleic acid construct further comprises a non-cancerous associated responsive element for regulating transcription of said anti-toxin.

27. The cell-free particle of claim 26, wherein said non-cancerous associated responsive element comprises the p53 wild type responsive element.

28. The cell-free particle of claim 2, wherein said first nucleic acid construct comprises four repeats of the PY2 sequence set forth by SEQ ID NO:2 being upstream and operably linked to the SV40 minimal promoter region set forth by SEQ ID NO:4, and a toxin coding sequence being downstream of and transcriptionally regulated by said SV40 minimal promoter region.

29. The cell-free particle of claim 2, wherein said second nucleic acid construct comprises the p53 wild type responsive element set forth by SEQ ID NO:14 being upstream and operably linked to the SV40 minimal promoter region set forth by SEQ ID NO:4, and an antitoxin coding sequence being downstream of and transcriptionally regulated by said SV40 minimal promoter region.

30. The cell-free particle of claim 29, wherein said second nucleic acid construct comprises about 17 repeats of said p53 wild type responsive element.

31. A pharmaceutical composition comprising the cell-free particle of claim 1 and a pharmaceutically acceptable carrier or diluents.

32. A method of treating cancer in a subject in need thereof, the method comprising administering to the subject a therapeutically effective amount of the pharmaceutical composition of claim 31, wherein cancer cells of the subject are characterized by expression of a tumor marker to which said targeting moiety is directed, thereby treating cancer in the subject.

33. The method of claim 32, wherein said cancer cells are characterized by hyper activity of said signaling as compared to non-cancerous cells of the same tissue, thereby treating the cancer.

34. A composition comprising the cell-free particle of claim 1 for use in the treatment of cancer, wherein cancer cells of the cancer are characterized by expression of a tumor marker to which said targeting moiety is directed and optionally wherein said cancer cells are characterized by hyper activity of said signaling as compared to non-cancerous cells of the same tissue.

35. The method of claim 32, wherein said cancer comprises colon cancer.

36. The method of claim 32, wherein said cancer comprises pancreatic cancer.

37. The method of claim 32, wherein said cancer comprises lung cancer.

38. The method of claim 32, wherein said tumor marker comprises CD24.

39. The method of claim 32, further comprising treating a subject having said cancer by a treatment selected from the group consisting of chemotherapy, biological therapy, radiotherapy, phototherapy, photodynamic therapy, surgery, nutritional therapy, ablative therapy, combined radiotherapy and chemotherapy, brachiotherapy, proton beam therapy, immunotherapy, cellular therapy and photon beam radiosurgical therapy.

40. The composition of claim 34, further comprising an agent suitable for a treatment selected from the group consisting of chemotherapy, biological therapy, photodynamic therapy, nutritional therapy, brachiotherapy, immunotherapy, and cellular therapy.

41. The cell-free particle of claim 1, further comprising an anti-cancer agent.

42. The cell-free particle of claim 41, wherein said anti-cancer agent is encapsulated in or conjugated to said particle.


SEQUENCE LISTING (62 sequences)

SEQ ID NO: 1 - DNA, 7 bp, Artificial sequence
  Ets binding site
caggaag

SEQ ID NO: 2 - DNA, 8 bp, Artificial sequence
  AP-1 binding site
tgactaag

SEQ ID NO: 3 - DNA, 15 bp, Artificial sequence
  PY2 sequence
caggaagtgactaag

SEQ ID NO: 4 - DNA, 203 bp, Artificial sequence
  SV40 minimal promoter
gcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 5 - DNA, 717 bp, Artificial sequence
  mCherry coding sequence
atgggaattcacgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 6 - DNA, 342 bp, Artificial sequence
  E. coli MazF ribonuclease
atggtaagccgatacgtacccgatatgggcgatctgatttgggttgattttgacccgacaaaaggtagcgagcaagctggacatcgtccagctgttgtcctgagtcctttcatgtacaacaacaaaacaggtatgtgtctgtgtgttccttgtacaacgcaatcaaaaggatatccgttcgaagttgttttatccggtcaggaacgtgatggcgtagcgttagctgatcaggtaaaaagtatcgcctggcgggcaagaggagcaacgaagaaaggaacagttgccccagaggaattacaactcattaaagccaaaattaacgtactgattggcaccggttag

SEQ ID NO: 7 - DNA, 588 bp, Artificial sequence
  IRES
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 8 - DNA, 657 bp, Artificial sequence
  Tetracycline repressor
atgagcagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaa

SEQ ID NO: 9 - DNA, 241 bp, Artificial sequence
  CMV minimal promoter+2xtetracycline operator
accaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccg

SEQ ID NO: 10 - DNA, 42 bp, Artificial sequence
  2xtetracycline operator
tccctatcagtgatagagatctccctatcagtgatagagatc

SEQ ID NO: 11 - DNA, 21 bp, Artificial sequence
  tetracycline operator
tccctatcagtgatagagatc

SEQ ID NO: 12 - DNA, 717 bp, Artificial sequence
  GFP coding sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 13 - DNA, 249 bp, Artificial sequence
  E. coli antitoxin MazE
atgatccacagtagcgtaaagcgttggggaaattcaccggcggtgcggatcccggctacgttaatgcaggcgctcaatctgaatattgatgatgaagtgaagattgacctggtggatggcaaattaattattgagccagtgcgtaaagagcccgtatttacgcttgctgaactggtcaacgacatcacgccggaaaacctccacgagaatatcgactggggagagccgaaagataaggaagtctggtaa

SEQ ID NO: 14 - DNA, 19 bp, Artificial sequence
  p53 wild type responsive element
cctgcctggacttgcctgg

SEQ ID NO: 15 - DNA, 325 bp, Artificial sequence
  RGCx17 (17 repeats of the p53 WT responsive element)
cctgcctggacttgcctggcctgcctggacttgcctggcctgcctggacttgcctggcctgcctggacttgcctggcctgcctggacttgcctggcctgcctggacttgcctggcctgcctggacttgcctggcctgcctggacttgcctggcctgcctggacttgcctggcccctgcctggacttgcctggcctgcctggacttgcctggcctgcctggacttgcctggcctgcctggacttgcctggcctgcctggacttgcctggcctgcctggacttgcctggcctgcctggacttgcctggcctgcctggacttgcctgg

SEQ ID NO: 16 - protein, 188 aa, Homo sapiens
  MISC_FEATURE (13)..(13): Xaa can be glycine or aspartic acid
MTEYKLVVVGAGXVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 17 - protein, 189 aa, homo sapiens
  MISC_FEATURE (61)..(61): Xaa can be Glutamine or lysine
MTEYKLVVVGAGGVGKSALTIQLIQNHFVDEYDPTIEDSYRKQVVIDGETCLLDILDTAGXEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 18 - protein, 189 aa, homo sapiens
  MISC_FEATURE (12)..(12): Xaa can be glycine or valine
MTEYKLVVVGAXGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 19 - DNA, 527 bp, Artificial sequence
  CMV minimal promoter (without the tetracycline operator)
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaga

SEQ ID NO: 20 - DNA, 1653 bp, Artificial sequence
  luciferase coding sequence
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 21 - DNA, 52 bp, Artificial sequence
  Hind-Cher-Forward primer
cttttgcaaaaagcttccaccatgggaattcacgtgagcaagggcgaggagg

SEQ ID NO: 22 - DNA, 49 bp, Artificial sequence
  Xba-Maz-Reverse primer
ccgccccgactctagactaaccggtgccaatcagtacgttaattttggc

SEQ ID NO: 23 - DNA, 4 bp, Artificial sequence
  ETS domain
ggaw

SEQ ID NO: 24 - DNA, 36 bp, Artificial sequence
  ToxI RNA repeated motif
aggtgatttgctacctttaagtgcagctagaaattc

SEQ ID NO: 25 - DNA, 21 bp, Artificial sequence
  Single strand DNA oligonucleotide
cccaagcaatggatgatttga

SEQ ID NO: 26 - DNA, 21 bp, Artificial sequence
  Single strand DNA oligonucleotide
ggcagaccagcatgacagatt

SEQ ID NO: 27 - DNA, 22 bp, Artificial sequence
  Single strand DNA oligonucleotide
tgagcagatcatgaagacaggg

SEQ ID NO: 28 - DNA, 19 bp, Artificial sequence
  Single strand DNA oligonucleotide
agagctggaagtcgagtgt

SEQ ID NO: 29 - DNA, 19 bp, Artificial sequence
  Single strand DNA oligonucleotide
tcaacgcacagtacgagcg

SEQ ID NO: 30 - DNA, 21 bp, Artificial sequence
  Single strand DNA oligonucleotide
caggcaaatgtgcaataccaa

SEQ ID NO: 31 - DNA, 21 bp, Artificial sequence
  Single strand DNA oligonucleotide
gcctataagaacgtggtgggc

SEQ ID NO: 32 - DNA, 23 bp, Artificial sequence
  Single strand DNA oligonucleotide
ccctcctacctctggttcttacg

SEQ ID NO: 33 - DNA, 20 bp, Artificial sequence
  Single strand DNA oligonucleotide
ccaggaggcactcacagagc

SEQ ID NO: 34 - DNA, 20 bp, Artificial sequence
  Single strand DNA oligonucleotide
ctcaacgtcgaccccgataa

SEQ ID NO: 35 - DNA, 20 bp, Artificial sequence
  Single strand DNA oligonucleotide
ccaccgcatctctacattca

SEQ ID NO: 36 - DNA, 21 bp, Artificial sequence
  Single strand DNA oligonucleotide
ggcattctgggagcttcatct

SEQ ID NO: 37 - DNA, 20 bp, Artificial sequence
  Single strand DNA oligonucleotide
gcggattagggcttcctctt

SEQ ID NO: 38 - DNA, 20 bp, Artificial sequence
  Single strand DNA oligonucleotide
gctcgatcctggatgaaacc

SEQ ID NO: 39 - DNA, 19 bp, Artificial sequence
  Single strand DNA oligonucleotide
gcaccttcacattcctctc

SEQ ID NO: 40 - DNA, 20 bp, Artificial sequence
  Single strand DNA oligonucleotide
gtaagggcaggagtcccatg

SEQ ID NO: 41 - DNA, 26 bp, Artificial sequence
  Single strand DNA oligonucleotide
ggttacagcaccatcagtaggtacag

SEQ ID NO: 42 - DNA, 21 bp, Artificial sequence
  Single strand DNA oligonucleotide
cctcgttgcttttctgctcaa

SEQ ID NO: 43 - DNA, 23 bp, Artificial sequence
  Single strand DNA oligonucleotide
ttgaatgtcagtcacttgggcat

SEQ ID NO: 44 - DNA, 18 bp, Artificial sequence
  Single strand DNA oligonucleotide
gcccttggacggcttttc

SEQ ID NO: 45 - DNA, 21 bp, Artificial sequence
  Single strand DNA oligonucleotide
acatctctgtcgtcgtcctcg

SEQ ID NO: 46 - DNA, 21 bp, Artificial sequence
  Single strand DNA oligonucleotide
caagtctggctcgttctcagt

SEQ ID NO: 47 - protein, 52 aa, Escherichia coli
MKLPRSSLVWCVLIVCLTLLIFTYLTRKSLCEIRYRDGHREVAAFMAYESGK

SEQ ID NO: 48 - DNA, 159 bp, Escherichia coli
atgaaactaccacgaagttcccttgtctggtgtgtgttgatcgtgtgtctcacactgttgatattcacttatctgacacgaaaatcgctgtgcgagattcgttacagagacggacacagggaggtggcggctttcatggcttacgaatccggtaagtag

SEQ ID NO: 49 - DNA, 225 bp, Escherichia coli
atgtggactagacatagggatgcctcgtggtggttaatgaaaattaacttactacggggctatcttctttctgccacacaacacggcaacaaaccaccttcacgtcatgaggcagaaagcctcaagcgccgggcacatcatagcccatatacctgcacgctgaccacactcactttccctgaaaataatccgctcattcagaccgttcacgggaaatccgtgtga

SEQ ID NO: 50 - protein, 74 aa, Escherichia coli
MWTRHRDASWWLMKINLLRGYLLSATQHGNKPPSRHEAESLKRRAHHSPYTCTLTTLTFPENNPLIQTVHGKSV

SEQ ID NO: 51 - protein, 111 aa, Unknown
  MULTISPECIES mRNA interferase MazF [Proteobacteria]
MVSRYVPDMGDLIWVDFDPTKGSEQAGHRPAVVLSPFMYNNKTGMCLCVPCTTQSKGYPFEVVLSGQERDGVALADQVKSIAWRARGATKKGTVAPEELQLIKAKINVLIG

SEQ ID NO: 52 - protein, 82 aa, Escherichia coli
MIHSSVKRWGNSPAVRIPATLMQALNLNIDDEVKIDLVDGKLIIEPVRKEPVFTLAELVNDITPENLHENIDWGEPKDKEVW

SEQ ID NO: 53 - DNA, 8 bp, Artificial sequence
  E2F binding site
tttcccgc

SEQ ID NO: 54 - DNA, 1152 bp, Artificial sequence
  nucleic acid encoding signal peptide, CD24 scFV and mVSVG
atgttcatgccctcctctttatcctatagcagctgggccacttgttggctgctctgttgtttaattatcctcgccaagaactcctccaccgacccttctcgtaatagcgacaccatgaaatgtttactctatttagcctttctgttcatcggagtgaactgcaagttcaccattgtgtttccccacaacggcaagcaagttcatctgcaagaaagcggccccggtctggtgaagccttcccagactttatctttaacatgtgccgtgagcggctacagcatctccagcggctacacttggcactggatcagacaaccccccggtaaaaccgtggagtggatcggctacatccagtataccggaagcaccagatacaaccccgctttaagaggcagactgacaatgtccgtcgacacaagcaagaaccagttctctttaaagctcatctccgtgaccgctgctgataccggagtctacttctgtgctcgtggcaccaccgctagctttgactactggggccaaggtacaacagtgaccgtgtcctccgccggctccgccggcggaggaggctctggcggcggcggcagcggcggtggcggcagcgatatcgtgatgacccaatcccccgactctttaaatgtgtctttaggcgagagggccacaattagatgtaggtccagccagtctttactgtactccagcgaccagaagaactatttaacttggtaccagcaaaagcccggtcagcctcctaagctgctcatcagctgggcctcccctagagccagcggcgtccccgatagattcagcggcagcggatccggcacagactttactttaaccatcagcagcctccaagctgaggacgtgggagtctactactgccagcagtactttatctaccctttaaccttcggagtgggaacaaagctggaactcaagggcgataccggactgtccaagaatcctatcgagctggtggaaggctggttctcctcttggaagtccagcattgcctccttcttctttattatcggtttaatcatcggtctgttcctcgtgctgagggtcggcatccacctctgcattaaactgaaacacaccaaaaaaagacaaatttacaccgatatcgagatgaatcgtctcggcaaa

SEQ ID NO: 55 - DNA, 195 bp, Artificial sequence
  nucleic acid encoding a signal peptide optimized for expression in mammalian cells
atgttcatgccctcctctttatcctatagcagctgggccacttgttggctgctctgttgtttaattatcctcgccaagaactcctccaccgacccttctcgtaatagcgacaccatgaaatgtttactctatttagcctttctgttcatcggagtgaactgcaagttcaccattgtgtttccccacaacggcaag

SEQ ID NO: 56 - DNA, 747 bp, Artificial sequence
  nucleic acid encoding CD24 scFV optimized for expression in mammalian cells
caagttcatctgcaagaaagcggccccggtctggtgaagccttcccagactttatctttaacatgtgccgtgagcggctacagcatctccagcggctacacttggcactggatcagacaaccccccggtaaaaccgtggagtggatcggctacatccagtataccggaagcaccagatacaaccccgctttaagaggcagactgacaatgtccgtcgacacaagcaagaaccagttctctttaaagctcatctccgtgaccgctgctgataccggagtctacttctgtgctcgtggcaccaccgctagctttgactactggggccaaggtacaacagtgaccgtgtcctccgccggctccgccggcggaggaggctctggcggcggcggcagcggcggtggcggcagcgatatcgtgatgacccaatcccccgactctttaaatgtgtctttaggcgagagggccacaattagatgtaggtccagccagtctttactgtactccagcgaccagaagaactatttaacttggtaccagcaaaagcccggtcagcctcctaagctgctcatcagctgggcctcccctagagccagcggcgtccccgatagattcagcggcagcggatccggcacagactttactttaaccatcagcagcctccaagctgaggacgtgggagtctactactgccagcagtactttatctaccctttaaccttcggagtgggaacaaagctggaactcaag

SEQ ID NO: 57 - DNA, 210 bp, Artificial sequence
  nucleic acid encoding mVSVG optimized for expression in mammalian cells
ggcgataccggactgtccaagaatcctatcgagctggtggaaggctggttctcctcttggaagtccagcattgcctccttcttctttattatcggtttaatcatcggtctgttcctcgtgctgagggtcggcatccacctctgcattaaactgaaacacaccaaaaaaagacaaatttacaccgatatcgagatgaatcgtctcggcaaa

SEQ ID NO: 58 - protein, 517 aa, Artificial sequence
  amino acid of VSV-G glycoprotein
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKMVSKGE

SEQ ID NO: 59 - protein, 750 aa, Artificial sequence
  amino acid of VSV-G glycoprotein with GFP
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGKMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 60 - protein, 65 aa, human
MFMPSSLSYSSWATCWLLCCLIILAKNSSTDPSRNSDTMKCLLYLAFLFIGVNCKFTIVFPHNGK

SEQ ID NO: 61 - protein, 249 aa, Artificial sequence
  amino acid of CD24 scFv
QVHLQESGPGLVKPSQTLSLTCAVSGYSISSGYTWHWIRQPPGKTVEWIGYIQYTGSTRYNPALRGRLTMSVDTSKNQFSLKLISVTAADTGVYFCARGTTASFDYWGQGTTVTVSSAGSAGGGGSGGGGSGGGGSDIVMTQSPDSLNVSLGERATIRCRSSQSLLYSSDQKNYLTWYQQKPGQPPKLLISWASPRASGVPDRFSGSGSGTDFTLTISSLQAEDVGVYYCQQYFIYPLTFGVGTKLELK

SEQ ID NO: 62 - protein, 70 aa, Artificial sequence
  amino acid of mVSV-G
GDTGLSKNPIELVEGWFSSWKSSIASFFFIIGLIIGLFLVLRVGIHLCIKLKHTKKRQIYTDIEMNRLGK